(19) United States
(12) Patent Application Publication
(10) Pub. No.: US 2010/0009905 A1
(43) Pub. Date: Jan. 14, 2010

(54) Compositions and Methods for Detection, Prognosis and Treatment of Colon Cancer

(76) Inventor: Roberto A. Macina, San Jose, CA (US)

Correspondence Address: LICATA & TYRRELL P.C., 66 E. MAIN STREET, MARLTON, NJ, 08053, US

(21) Appl. No.: 12/294,288

(22) PCT Filed: Mar. 26, 2007
(86) PCT No.: PCT/US07/64876

§ 371 Date: Sep. 9, 2009

Related U.S. Application Data
(60) Provisional application No. 60/785,536, filed on Mar. 24, 2006.

(51) Int. Cl. A61K 38/16; C12Q 1/68; C07H 21/04; C07H 21/02; G01N 33/53; A61K 31/7088

(52) U.S. Cl. 514/12; 435/6; 536/23.1; 536/24.5; 435/7.1; 514/44 A; 514/44 R

(57) ABSTRACT

The present invention relates to methods of detection, prognosis and treatment of colon cancer using a plurality genes or gene products present in normal and neoplastic cells, tissues and bodily fluids. Additional uses include identifying, monitoring, staging, imaging and treating colon cancer and non-cancerous diseases of the colon as well as determining the effectiveness of therapies alone or in combination for an individual.

This patent application claims the benefit of priority from U.S. Provisional Application Ser. No. 60/785,536, filed Mar. 24, 2006, teachings of which are herein incorporated by reference in their entirety.

### FIELD OF THE INVENTION

The present invention relates to methods of detection, prognosis and treatment of colon cancer using a plurality genes or gene products present in normal and neoplastic cells, tissues and bodily fluids. Gene products relate to compositions comprising the nucleic acids, polypeptides, post translational modifications (PTMs), variants, and derivatives of the invention and methods for the use of these compositions. Additional uses include identifying, monitoring, staging, imaging and treating cancer and non-cancerous disease states in the colon as well as determining the effectiveness of therapies alone or in combination for an individual.

### BACKGROUND OF THE INVENTION

### Colon Cancer

Colorectal cancer is the second most common cause of cancer death in the United States and the third most prevalent cancer in both men and women. M. L. Davila & A. D. Davila, Screening for Colon and Rectal Cancer, in Colon and Rectal Cancer 47 (Peter S. Edelstein ed., 2000). Colorectal cancer is categorized as a digestive system cancer by the American Cancer Society (ACS) which also includes cancers of the esophagus, stomach, small intestine, anus, anal canal, anorectum, liver and intrahepatic bile duct, gallbladder and other biliary, pancreas, and other digestive organs. The ACS estimates that there will be about 253,500 new cases of digestive system cancers in 2005 in the United States alone. Digestive system cancers will cause an estimated 136,060 deaths combined in the United States in 2005. Specifically, The ACS estimates that there will be about 104,950 new cases of colon cancer, 40,340 new cases of rectal cancer and 5,420 new cases of small intestine cancer in the 2005 in the United States alone. Colon, rectal and small intestine cancers will cause an estimated 57,360 deaths combined in the United States in 2005. ACS Website: cancer with the extension .org of the world wide web. Nearly all cases of colorectal cancer arise from adenomatous polyps, some of which mature into large polyps, undergo abnormal growth and development, and ultimately progress into cancer. Davila at 55-56. This progression would appear to take at least 10 years in most patients, rendering it a readily treatable form of cancer if diagnosed early, when the cancer is localized. Davila at 56; Walter J. Burdette, Cancer: Etiology, Diagnosis, and Treatment 125 (1998).

Although our understanding of the etiology of colon cancer is undergoing continual refinement, extensive research in this area points to a combination of factors, including age, hereditary and nonhereditary conditions, and environmental/dietary factors. Age is a key risk factor in the development of colorectal cancer, Davila at 48, with men and women over 40 years of age become increasingly susceptible to that cancer, Burdette at 126. Incidence rates increase considerably in each subsequent decade of life. Davila at 48. A number of hereditary and nonhereditary conditions have also been linked to a heightened risk of developing colorectal cancer, including familial adenomatous polyposis (FAP), hereditary nonpolyposis colorectal cancer (Lynch syndrome or HNPCC), a personal and/or family history of colorectal cancer or adenomatous polyps, inflammatory bowel disease, diabetes mellitus, and obesity. Id. at 47; Henry T. Lynch & Jane F. Lynch, Hereditary Nonpolyposis Colorectal Cancer (Lynch Syndromes), in Colon and Rectal Cancer 67-68 (Peter S. Edelstein ed., 2000).

Environmental/dietary factors associated with an increased risk of colorectal cancer include a high fat diet, intake of high dietary red meat, and sedentary lifestyle. Davila at 47; Reddy, B. S., Prev. Med. 16(4): 460-7 (1987). Conversely, environmental/dietary factors associated with a reduced risk of colorectal cancer include a diet high in fiber, folic acid, calcium, and hormone-replacement therapy in post-menopausal women. Davila at 50-55. The effect of antioxidants in reducing the risk of colon cancer is unclear. Davila at 53.

Because colon cancer is highly treatable when detected at an early, localized stage, screening should be a part of routine care for all adults starting at age 50, especially those with first-degree relatives with colorectal cancer. One major advantage of colorectal cancer screening over its counterparts in other types of cancer is its ability to not only detect precancerous lesions, but to remove them as well. Davila at 56. The key colorectal cancer screening tests in use today are fecal occult blood test, sigmoidoscopy, colonoscopy, double-contrast barium enema, and the carcinoembryonic antigen (CEA) test. Burdette at 125; Davila at 56. Virtual colonoscopy is an emerging colorectal screening test that is sensitive and less invasive than traditional colonoscopy. Scharling E S et al, Semin Roentgenol. 1996 April; 31(2):142-53. Johnson C D et al Gut. 1999 March; 44(3):301-5. Fenlon H M et al., N Engl J Med. 1999 Nov. 11; 341(20): 1496-503. Selcuk D et al. Turk J Gastroenterol. 2006 December; 17(4):288-293.

The fecal occult blood test (FOBT) screens for colorectal cancer by detecting the amount of blood in the stool, the premise being that neoplastic tissue, particularly malignant tissue, bleeds more than typical mucosa, with the amount of bleeding increasing with polyp size and cancer stage. Davila at 56-57. While effective at detecting early stage tumors, FOBT is unable to detect adenomatous polyps (premalignant lesions), and, depending on the contents of the fecal sample, is subject to rendering false positives. Davila at 56-59. Sigmoidoscopy and colonoscopy, by contrast, allow direct visualization of the bowel, and enable one to detect, biopsy, and remove adenomatous polyps. Davila at 59-60, 61. Despite the advantages of these procedures, there are accompanying downsides: sigmoidoscopy, by definition, is limited to the sigmoid colon and below, colonoscopy is a relatively expensive procedure, and both share the risk of possible bowel perforation and hemorrhaging. Davila at 59-60. Double-contrast barium enema (DCBE) enables detection of lesions better than FOBT, and almost as well a colonoscopy, but it may be limited in evaluating the winding rectosigmoid region. Davila at 60. The CEA blood test, which involves screening the blood for carcinoembryonic antigen, shares the downside of FOBT, in that it is of limited utility in detecting colorectal cancer at an early stage. Burdette at 125.

Once colon cancer has been diagnosed, treatment decisions are typically made in reference to the stage of cancer progression. A number of techniques are employed to stage the cancer (some of which are also used to screen for colon cancer), including pathologic examination of resected colon, sigmoidoscopy, colonoscopy, and various imaging techniques. AJCC Cancer Staging Handbook 84 (Irvin D. Fleming et al. eds., 5^{th} ed. 1998); Montgomery, R. C. and Ridge, J. A., Semin. Surg. Oncol. 15(3): 143-150 (1998). Moreover, chest films, liver functionality tests, and liver scans are employed to determine the extent of metastasis. Fleming at 84. While computerized tomography and magnetic resonance imaging are useful in staging colorectal cancer in its later stages, both have unacceptably low staging accuracy for identifying early stages of the disease, due to the difficulty that both methods have in (1) revealing the depth of bowel wall tumor infiltration and (2) diagnosing malignant adenopathy. Thoeni, R. F., Radiol. Clin. N. Am. 35(2): 457-85 (1997). Rather, techniques such as transrectal ultrasound (TRUS) are preferred in this context, although this technique is inaccurate with respect to detecting small lymph nodes that may contain metastases. David Blumberg & Frank G. Opelka, Neoadjuvant and Adjuvant Therapy for Adenocarcinoma of the Rectum, in Colon and Rectal Cancer 316 (Peter S. Edelstein ed., 2000).

Several classification systems have been devised to stage the extent of colorectal cancer, including the Dukes' system and the more detailed International Union against Cancer-American Joint Committee on Cancer TNM staging system, which is considered by many in the field to be a more useful staging system. Burdette at 126-27. The TNM system, which is used for either clinical or pathological staging, is divided into four stages, each of which evaluates the extent of cancer growth with respect to primary tumor (T), regional lymph nodes (N), and distant metastasis (M). Fleming at 84-85. The system focuses on the extent of tumor invasion into the intestinal wall, invasion of adjacent structures, the number of regional lymph nodes that have been affected, and whether distant metastasis has occurred. Fleming at 81.

Stage 0 is characterized by in situ carcinoma (Tis), in which the cancer cells are located inside the glandular basement membrane (intraepithelial) or lamina propria (intramucosal). In this stage, the cancer has not spread to the regional lymph nodes (N0), and there is no distant metastasis (M0). In stage I, there is still no spread of the cancer to the regional lymph nodes and no distant metastasis, but the tumor has invaded the submucosa (T1) or has progressed further to invade the muscularis propria (T2). Stage II also involves no spread of the cancer to the regional lymph nodes and no distant metastasis, but the tumor has invaded the subserosa, or the nonperitonealized pericolic or perirectal tissues (T3), or has progressed to invade other organs or structures, and/or has perforated the visceral peritoneum (T4). Stage III is characterized by any of the T substages, no distant metastasis, and either metastasis in 1 to 3 regional lymph nodes (N1) or metastasis in four or more regional lymph nodes (N2). Lastly, stage 1V involves any of the T or N substages, as well as distant metastasis. Fleming at 84-85; Burdette at 127.

Currently, pathological staging of colon cancer is preferable over clinical staging as pathological staging provides a more accurate prognosis. Pathological staging typically involves examination of the resected colon section, along with surgical examination of the abdominal cavity. Fleming at 84. Clinical staging would be a preferable method of staging were it at least as accurate as pathological staging, as it does not depend on the invasive procedures of its counterpart.

Turning to the treatment of colorectal cancer, surgical resection results in a cure for roughly 50% of patients. Irradiation is used both preoperatively and postoperatively in treating colorectal cancer. Chemotherapeutic agents, particularly 5-fluorouracil, are also powerful weapons in treating colorectal cancer. Other agents include irinotecan and floxuridine, cisplatin, levamisole, methotrexate, interferon-α, and leucovorin. Burdette at 125, 132-33. Nonetheless, thirty to forty percent of patients will develop a recurrence of colon cancer following surgical resection, which in many patients is the ultimate cause of death. Wayne De Vos, Follow-up After Treatment of Colon Cancer, Colon and Rectal Cancer 225 (Peter S. Edelstein ed., 2000). Accordingly, colon cancer patients must be closely monitored to determine response to therapy and to detect persistent or recurrent disease and metastasis.

Approximately 75% of patients with colorectal cancer present with localized disease of which after curative surgery approximately 40% experience disease relapse leading to morbidity and eventual mortality. In patients with resectable stage III colorectal cancer, adjuvant therapy improves disease-free survival by 35% and overall survival by 22%. The successful use of adjuvant therapy in stage II colorectal cancer remains controversial. Patients with stage II colorectal have a 5-year survival rate of 75%, which indicates that the majority of patients are cured by surgery alone. On the other hand, 40% of these patients will develop recurrent disease within their lifetime; therefore, there is a need to identify which of these patients with stage II colorectal cancer would benefit from adjuvant therapy. Molecular profiling of tumors may identify patients who are more likely to benefit from adjuvant therapy. This would enable the clinician to tailor treatment according to an individual patient and tumor profile. In colorectal cancer, a limited number of predictive markers have been identified to date and there is a need for multiple marker testing in order to improve response rates and decrease toxicity in colorectal cancer patients. W. L. Allen and P. G. Johnston, Role of genomic markers in colorectal cancer treatment, Journal of Clinical Oncology 23, 4545.

The next few paragraphs describe some of molecular bases of colon cancer. In the case of FAP, the tumor suppressor gene APC (adenomatous polyposis coli), chromosomally located at 5q21, has been either inactivated or deleted by mutation. Alberts et al., Molecular Biology of the Cell 1288 (3d ed. 1994). The APC protein plays a role in a number of functions, including cell adhesion, apoptosis, and repression of the c-myc oncogene. N. R. Hall & R. D. Madoff, Genetics and the Polyp-Cancer Sequence, Colon and Rectal Cancer 8 (Peter S. Edelstein, ed., 2000). Of those patients with colorectal cancer who have normal APC genes, over 65% have such mutations in the cancer cells but not in other tissues. Alberts et al., supra at 1288. In the case of HPNCC, patients manifest abnormalities in the tumor suppressor gene HNPCC, but only about 15% of tumors contain the mutated gene. Id. A host of other genes have also been implicated in colorectal cancer, including the K-ras, N-ras, H-ras and c-myc oncogenes, and the tumor suppressor genes DCC (deleted in colon carcinoma) and p53. Hall & Madoff, supra at 8-9; Alberts et al., supra at 1288.

Abnormalities in Wg/Wnt signal transduction pathway are also associated with the development of colorectal carcinoma. Taipale, J. and Beachy, P. A. Nature 411: 349-354 (2001). Wnt1 is a secreted protein gene originally identified within mouse mammary cancers by its insertion into the mouse mammary tumor virus (MMTV) gene. The protein is homologous to the wingless (Wg) gene product of Drosophila, in which it functions as an important factor for the determination of dorsal-ventral segmentation and regulates the formation of fly imaginal discs. Wg/Wnt pathway controls cell proliferation, death and differentiation, Taipal (2001). There are at least 13 members in the Wnt family. These proteins have been found expressed mainly in the central nervous system (CNS) of vertebrates as well as other tissues such as mammary and intestine. The Wnt proteins are the ligands for a family of seven transmembrane domain receptors related to the Frizzled gene product in Drosophila. Binding Wnt to Frizzled stimulates the activity of the downstream target, Dishevelled, which in turn inactivates the glycogen synthetase kinase 3β (GSK3β), Taipal (2001). Usually active GSK3β will form a complex with the adenomatous polyposis coli (APC) protein and phosphorylate another complex member, β-catenin. Once phosphorylated, β-catenin is directed to degradation through the ubiquitin pathway. When GSK3β or APC activity is down regulated, β-catenin is accumulated in the cytoplasm and binds to the T-cell factor or lymphocyte excitation factor (Tcf/Lef) family of transcriptional factors. Binding of β-catenin to Tcf releases the transcriptional repression and induces gene transcription. Among the genes regulated by β-catenin are a transcriptional repressor Engrailed, a transforming growth factor-β (TGF-β) family member Decapentaplegic, and the cytokine Hedgehog in Drosophila. β-Catenin also involves in regulating cell adhesion by binding to α-catenin and E-cadherin. On the other hand, binding of β-catenin to these proteins controls the cytoplasmic β-catenin level and its complexing with TCF, Taipal (2001). Growth factor stimulation and activation of c-src or v-src also regulate β-catenin level by phosphorylation of α-catenin and its related protein, p120^{cas}. When phosphorylated, these proteins decrease their binding to E-cadherin and β-catenin resulting in the accumulation of cytoplasmic β-catenin. Reynolds, A. B. et al. Mol. Cell. Biol. 14: 8333-8342 (1994). In colon cancer, c-src enzymatic activity has been shown increased to the level of v-src. Alternation of components in the Wg/Wnt pathway promotes colorectal carcinoma development. The best known modifications are to the APC gene. Nicola S et al. Hum. Mol. Genet. 10:721-733 (2001). This germline mutation causes the appearance of hundreds to thousands of adenomatous polyps in the large bowel. It is the gene defect that accounts for the autosomally dominantly inherited FAP and related syndromes. The molecular alternations that occur in this pathway largely involve deletions of alleles of tumor-suppressor genes, such as APC, p53 and Deleted in Colorectal Cancer (DCC), combined with mutational activation of proto-oncogenes, especially c-Ki-ras. Aoki, T. et al. Human Mutat. 3: 342-346 (1994). All of these lead to genomic instability in colorectal cancers.

Another source of genomic instability in colorectal cancer is the defect of DNA mismatch repair (MMR) genes. Human homologues of the bacterial mutHLS complex (hMSH2, hMLH1, hPMS1, hPMS2 and hMSH6), which is involved in the DNA mismatch repair in bacteria, have been shown to cause the HNPCC (about 70-90% HNPCC) when mutated. Modrich, P. and Lahue, R. Ann Rev. Biochem. 65: 101-133 (1996); and Peltomäki, P. Hum. Mol. Genet. 10: 735-740 (2001). The inactivation of these proteins leads to the accumulation of mutations and causes a genetic instability that represents errors in the accurate replication of the repetitive mono-, di-, tri- and tetra-nucleotide repeats (microsatellite regions), which are scattered throughout the genome called microsatellite instability (MSI). Jass, J. R. et al. J Gastroenterol Hepatol 17: 17-26 (2002). Like in the classic FAP, mutational activation of c-Ki-ras is also required for the promotion of MSI in the alternative HNPCC. Mutations in other proteins such as the tumor suppressor protein phosphatase PTEN (Zhou, X. P. et al. Hum. Mol. Genet. 11: 445-450 (2002)), BAX (Buttler, L. M. Aus. N. Z. J. Surg. 69: 88-94 (1999)), Caspase-5 (Planck, M. Cancer Genet Cytogenet. 134: 46-54 (2002)), TGFβ-RII (Fallik, D. et al. Gastroenterol Clin Biol. 24: 917-22 (2000)) and IGFII-R (Giovannucci E. J. Nutr. 131: 3109S-20S (2001)) have also been found in some colorectal tumors possibly as the cause of MMR defect.

Some tyrosine kinases have been shown up-regulated in colorectal tumor tissues or cell lines like HT29. Skoudy, A. et al. Biochem J. 317 (Pt 1): 279-84 (1996). Focal adhesion kinase (FAK) and its up-stream kinase c-src and c-yes in colonic epithelia cells may play an important role in the promotion of colorectal cancers through the extracellular matrix (ECM) and integrin-mediated signaling pathways. Jessup, J. M. et al., The molecular biology of colorectal carcinoma, in: The Molecular Basis of Human Cancer, 251-268 (Coleman W. B. and Tsongalis G. J. Eds. 2002). The formation of c-src/FAK complexes may coordinately deregulate VEGF expression and apoptosis inhibition. Recent evidences suggest that a specific signal-transduction pathway for cell survival that implicates integrin engagement leads to FAK activation and thus activates PI-3 kinase and akt. In turn, akt phosphorylates BAD (a pro-apoptotic member of the Bcl-2 family), and blocks apoptosis in epithelial cells. The activation of c-src in colon cancer may induce VEGF expression through the hypoxia pathway. Other genes that may be implicated in colorectal cancer include Cox enzymes (Ota, S. et al. Aliment Pharmacol. Ther. 16 (Suppl 2): 102-106 (2002)), estrogen (al-Azzawi, F. and Wahab, M. Climacteric 5: 3-14 (2002)), peroxisome proliferator-activated receptor-γ (PPAR-γ) (Gelman, L. et al. Cell Mol Life Sci. 55: 932-943 (1999)), IGF-I (Giovannucci (2001)), thymine DNA glycosylase (TDG) (Hardeland, U. et al. Prog. Nucleic Acid Res. Mol. Biol. 68: 235-253 (2001)) and EGF (Mendelsohn, J. Endocrine-Related Cancer 8: 3-9 (2001)).

Gene deletion and mutation are not the only causes for development of colorectal cancers. Epigenetic silencing by DNA methylation also accounts for the lost of function of colorectal cancer suppressor genes. A strong association between MSI and CpG island methylation has been well characterized in sporadic colorectal cancers with high MSI but not in those of hereditary origin. In one experiment, DNA methylation of MLH1, CDKN2A, MGMT, THBS1, RARB, APC, and p14ARF genes has been shown in 80%, 55%, 23%, 23%, 58%, 35%, and 50% of 40 sporadic colorectal cancers with high MSI respectively. Yamamoto, H. et al. Genes Chromosomes Cancer 33: 322-325 (2002); and Kim, K. M. et al. Oncogene. 12; 21(35): 5441-9 (2002). Carcinogen metabolism enzymes such as GST, NAT, CYP and MTHFR are also associated with an increased or decreased colorectal cancer risk. Pistorius, S. et al. Kongressbd Dtsch Ges Chir Kongr 118: 820-824 (2001); and Potter, J. D. J. Natl. Cancer Inst. 91: 916-932 (1999).

From the foregoing, it is clear that procedures used for detecting, diagnosing, monitoring, staging, prognosticating, and preventing the recurrence of colorectal cancer are of critical importance to the outcome of the patient. Moreover, current procedures, while helpful in each of these analyses, are limited by their specificity, sensitivity, invasiveness, and/or their cost. As such, highly specific and sensitive procedures that would operate by way of detecting novel markers in cells, tissues, or bodily fluids, with minimal invasiveness and at a reasonable cost, would be highly desirable.

Accordingly, there is a great need for more sensitive and accurate methods for predicting whether a person is likely to develop colorectal cancer, for diagnosing colorectal cancer, for monitoring the progression of the disease, for staging the colorectal cancer, for determining whether the colorectal cancer has metastasized, and for imaging the colorectal cancer. Following accurate diagnosis, there is also a need for less invasive and more effective treatment of colorectal cancer.

### Angiogenesis in Cancer

Growth and metastasis of solid tumors are also dependent on angiogenesis. Folkman, J., Cancer Research, 46: 467-473 (1986); Folkman, J., Journal of the National Cancer Institute, 82: 4-6 (1989). It has been shown, for example, that tumors which enlarge to greater than 2 mm must obtain their own blood supply and do so by inducing the growth of new capillary blood vessels. Once these new blood vessels become embedded in the tumor, they provide a means for tumor cells to enter the circulation and metastasize to distant sites such as liver, lung or bone. Weidner, N., et al., The New England Journal of Medicine, 324(1): 1-8 (1991).

Angiogenesis, defined as the growth or sprouting of new blood vessels from existing vessels, is a complex process that primarily occurs during embryonic development. The process is distinct from vasculogenesis, in that the new endothelial cells lining the vessel arise from proliferation of existing cells, rather than differentiating from stem cells. The process is invasive and dependent upon proteolysis of the extracellular matrix (ECM), migration of new endothelial cells, and synthesis of new matrix components. Angiogenesis occurs during embryogenic development of the circulatory system; however, in adult humans, angiogenesis only occurs as a response to a pathological condition (except during the reproductive cycle in women).

Under normal physiological conditions in adults, angiogenesis takes place only in very restricted situations such as hair growth and wounding healing. Auerbach, W. and Auerbach, R., Pharmacol Ther. 63(3):265-3 11 (1994); Ribatti et al., Haematologica 76(4):3 11-20 (1991); Risau, Nature 386(6626):67 1-4 (1997). Angiogenesis progresses by a stimulus which results in the formation of a migrating column of endothelial cells. Proteolytic activity is focused at the advancing tip of this “vascular sprout”, which breaks down the ECM sufficiently to permit the column of cells to infiltrate and migrate. Behind the advancing front, the endothelial cells differentiate and begin to adhere to each other, thus forming a new basement membrane. The cells then cease proliferation and finally define a lumen for the new arteriole or capillary.

Unregulated angiogenesis has gradually been recognized to be responsible for a wide range of disorders, including, but not limited to, cancer, cardiovascular disease, rheumatoid arthritis, psoriasis and diabetic retinopathy. Folkman, Nat. Med. 1(1):27-31 (1995); Isner, Circulation 99(13): 1653-5 (1999); Koch, Arthritis Rheum. 41(6):951-62 (1998); Walsh, Rheumatology (Oxford) 38(2):103-12 (1999); Ware and Simons, Nat. Med. 3(2): 158-64 (1997).

Of particular interest is the observation that angiogenesis is required by solid tumors for their growth and metastases. Folkman, 1986 supra; Folkman, J. Natl. Cancer Inst., 82(1) 4-6 (1990); Folkman, Semin. Cancer Biol. 3(2):65-71 (1992); Zetter, Annu. Rev. Med. 49:407-24 (1998). A tumor usually begins as a single aberrant cell which can proliferate only to a size of a few cubic millimeters due to the distance from available capillary beds, and it can stay dormant without further growth and dissemination for a long period of time. Some tumor cells then switch to the angiogenic phenotype to activate endothelial cells, which proliferate and mature into new capillary blood vessels. These newly formed blood vessels not only allow for continued growth of the primary tumor, but also for the dissemination and recolonization of metastatic tumor cells. The precise mechanisms that control the angiogenic switch is not well understood; but it is believed that neovascularization of tumor mass results from the net balance of a multitude of angiogenesis stimulators and inhibitors, Folkman, 1995, supra.

A potent angiogenesis inhibitor is endostatin identified by O'Reilly and Folkman. O'Reilly et al., Cell 88(2):277-85 (1997); O'Reilly et al., Cell 79(2):3 15-28 (1994). Its discovery was based on the phenomenon that certain primary tumors can inhibit the growth of distant metastases. O'Reilly and Folkman hypothesized that a primary tumor initiates angiogenesis by generating angiogenic stimulators in excess of inhibitors. However, angiogenic inhibitors, by virtue of their longer half life in the circulation, reach the site of a secondary tumor in excess of the stimulators. The net result is the growth of primary tumor and inhibition of secondary tumor. Endostatin is one of a growing list of such angiogenesis inhibitors produced by primary tumors. It is a proteolytic fragment of a larger protein: endostatin is a 20 kDa fragment of collagen XVIII (amino acid H1132-K1315 in murine collagen XVIII). Endostatin has been shown to specifically inhibit endothelial cell proliferation in vitro and block angiogenesis in vivo. More importantly, administration of endostatin to tumor-bearing mice leads to significant tumor regression, and no toxicity or drug resistance has been observed even after multiple treatment cycles. Boehm et al., Nature 390(6658):404-407 (1997). The fact that endostatin targets genetically stable endothelial cells and inhibits a variety of solid tumors makes it a very attractive candidate for anticancer therapy. Fidler and Ellis, Cell 79(2):185-8 (1994); Gastl et al., Oncology 54(3):177-84 (1997); Hinsbergh et al., Ann. Oncol. 10 Suppl. 4:60-3 (1999). In addition, angiogenesis inhibitors have been shown to be more effective when combined with radiation and chemotherapeutic agents. Klement, J. Clin. Invest., 105(8) R15-24 (2000). Browder, Cancer Res. 6-(7) 1878-86 (2000); Arap et al., Science 279(5349):377-80 (1998); Mauceri et al., Nature 394(6690):287-91 (1998).

### SUMMARY OF THE INVENTION

In one aspect, the invention concerns a method for determining the prognosis for an individual having colon cancer where the expression level of a plurality of gene products in Table 2a is determined, and where the differential expression of a plurality of gene products relative to a control is indicative of the individual's prognosis.

In a particular embodiment, the expression level of a plurality of gene products of the genes in Table 2b is also determined, and the differential expression of a plurality of gene products relative to a control is indicative of the individual's prognosis.

In another particular embodiment, the plurality of gene products comprises at least two, or at least four, or at least six, or at least eight gene products.

In another embodiment, the plurality of gene products are selected from the group comprising CA1, ITLN1, TSPAN1, CYR61, CXCL12, C20orf52, DPEP1, REGIV, NOX1, CEACAM5, FAM3D, OLFM4, HOXB9, SPP1, URCC, CEACAM6, AGR2, GDF15, SPON2, CCL20, C10orf35, SCD, TH1L, LCN2, MMP9, TYMS, TK1, DTYMK, CD44, NME1, MYBL2, TSPN6, HARS2, STAT6, GAL4, CA1, PIGR, REG3A, PACAP, NDRG1 and KRT20. In another embodiment, the over-expression of gene products are indicative of a poor prognosis. In a further specific embodiment, the over-expression of gene products are indicative of a poor prognosis. In another specific embodiment, the under-expression of gene products are indicative of a poor prognosis.

In another embodiment, the over-expression of gene products selected from the group comprising CA1, ITLN1, TSPAN1, CYR61 and CXCL12 and/or the under-expression of gene products selected from the group comprising C20orf52 and DPEP1 are indicative of a good prognosis. In a further embodiment, the over-expression of gene products selected from the group comprising REGIV, NOX1, CEACAM5, C20orf52, FAM3D, OLFM4, HOXB9, SPP1, URCC, CEACAM6, AGR2, GDF15, CCL20, C10orf35, SCD, TH1L, LCN2, MMP9, TYMS, TK1, DTYMK, CD44, NME1, MYBL2, DPEP1, TSPN6, HARS2 and STAT6 and/or the under-expression of gene products selected from the group comprising GAL4, CA1, PIGR, REG3A, PACAP, CYR61, NDRG1, CXCL12 and KRT20 are indicative of a poor prognosis.

In a particular embodiment, the gene product is RNA. In a further embodiment, the gene product expression level is determined by quantitative PCR.

In another particular embodiment, the gene product is a polypeptide. In a further embodiment, the gene product expression level is determined by an assay comprising one or more antibodies.

In another particular embodiment, the sample of gene products is selected from the group consisting of tissues, cells and bodily fluids. In a further embodiment, the sample of gene products is selected where the tissues or cells are from a fixed, waxed, embedded specimen from said individual.

In another aspect, the invention provides a method for improving the prognosis for an individual which comprises modulating levels of a plurality of gene products of Table 2a.

In a particular embodiment, the plurality of gene products comprises at least two, or at least four, or at least six, or at least eight gene products.

In another embodiment, modulating levels of gene products comprises increasing levels of gene products whose over-expression is associated with a good prognosis. In a further embodiment, the method includes increasing levels of gene products whose over-expression is associated with a good prognosis where the gene products are selected from the group comprising the gene products of Table 2a.

In another embodiment, modulating levels of gene products comprises decreasing levels of gene products whose under-expression is associated with a good prognosis. In a further embodiment, the method includes decreasing levels of gene products whose under-expression is associated with a good prognosis where the gene products are selected from the group comprising the gene products of Table 2a.

In another embodiment, modulating levels of gene products comprises decreasing levels of gene products whose over-expression is associated with a poor prognosis. In another embodiment, modulating levels of gene products comprises increasing levels of gene products whose under-expression is associated with a poor prognosis.

In another embodiment, the individual is administered an appropriate agonist or antagonist for a gene product of Table 2a which will improve the prognosis of the individual.

The invention further concerns an isolated nucleic acid molecule comprising (a) a nucleic acid molecule consisting essentially of a nucleic acid sequence that encodes an amino acid sequence of the gene products in Table 7; (b) a nucleic acid molecule that selectively hybridizes to the nucleic acid molecule of (a); or (c) a nucleic acid molecule having at least 95% sequence identity to the nucleic acid molecule of (a).

In a particular embodiment, the nucleic acid molecule is cDNA, genomic DNA, RNA, a mammalian nucleic acid molecule, or a human nucleic acid molecule.

The invention further concerns a set of three isolated nucleic acid molecules wherein: (a) each nucleic acid molecule consists essentially of a nucleic acid sequence encoding a portion of gene product described in Table 2a or Table 2b and (i) the first nucleic acid molecule is a forward primer 15 to 30 base pairs in length; (ii) the second nucleic acid molecule is reverse primer 15 to 30 base pairs in length; and (iii) the third nucleic acid molecule is a probe 15-30 base pairs in length; such that the forward primer and reverse primer produce an amplicon detectable by the probe wherein the amplicon could bridge two exons and is 60 to 100 base pairs in length; preferably 70 to 90 base pairs in length; (b) a nucleic acid molecule that selectively hybridizes to one of the three nucleic acid molecules of (a); or (c) a nucleic acid molecule having at least 95% sequence identity to one of the three nucleic acid molecules of (a). These three isolated nucleic acid molecules produce and detect an amplicon from an nucleic acid molecule comprising a nucleic acid molecule consisting essentially of a nucleic acid sequence that encodes an amino acid sequence of the gene products in Table 7.

In another aspect, the invention concerns a method for determining the presence of a gene product of Table 2a in a sample, comprising the steps of: (a) contacting the sample with the nucleic acid molecule of Table 7 under conditions in which the nucleic acid molecule will selectively hybridize to a gene product of Table 2a; and (b) detecting hybridization of the nucleic acid molecule to a gene product of Table 2a in the sample, wherein the detection of the hybridization indicates the presence of a gene product of Table 2a in the sample.

In another aspect, the invention concerns a method for determining the presence of cancer specific protein in a sample, comprising the steps of: (a) contacting the sample with a suitable reagent under conditions in which the reagent will selectively interact with a cancer specific protein comprising an amino acid sequence with at least 95% sequence identity to a polypeptide encoded by a gene product in Table 2a; and (b) detecting the interaction of the reagent with any cancer specific protein in the sample, wherein the detection of the binding indicates the presence of the cancer specific protein in the sample.

Another aspect of the invention concerns a method for diagnosing or monitoring the presence and/or metastases of colon cancer in an individual, comprising the steps of: (a) determining an amount of (i) a nucleic acid molecule consisting essentially of a nucleic acid sequence that encodes an amino acid sequence of a gene product in Table 2a; (ii) a nucleic acid molecule consisting essentially of a nucleic acid sequence of a gene product in Table 2a; (iii) a nucleic acid molecule consisting essentially of a nucleic acid sequence of Table 7; (iv) a nucleic acid molecule that selectively hybridizes to the nucleic acid molecule of (i), (ii) or (iii); (v) a nucleic acid molecule having at least 95% sequence identity to the nucleic acid molecule of (i), (ii) or (iii); (vi) a polypeptide comprising an amino acid sequence with at least 95% sequence identity to the polypeptide encoded by a gene product in Table 2a; or (vii) a polypeptide comprising an amino acid sequence encoded by a nucleic acid molecule having at least 95% sequence identity to a nucleic acid molecule comprising a nucleic acid sequence of a gene product of Table 2a; and (b) comparing the amount of the determined nucleic acid molecule or the polypeptide in the sample of the individual to the amount of the same nucleic acid molecule or polypeptide in a normal control; wherein a difference in the amount of the nucleic acid molecule or the polypeptide in the sample compared to the amount of the nucleic acid molecule or the polypeptide in the normal control is associated with the presence and/or metastases of colon cancer.

In another aspect, the invention concerns a kit for detecting a risk of cancer or presence of cancer in a individual, wherein the kit comprises a means for determining the presence of: (a) a nucleic acid molecule consisting essentially of a nucleic acid sequence that encodes an amino acid sequence of a polypeptide encoded by a gene product in Table 2a or 2b; (b) a nucleic acid molecule consisting essentially of a nucleic acid sequence of a gene product in Table 2a or 2b; (c) a nucleic acid molecule consisting essentially of a nucleic acid sequence of Table 7; (d) a nucleic acid molecule that selectively hybridizes to the nucleic acid molecule of (a), (b) or (c); (e) a nucleic acid molecule having at least 95% sequence identity to the nuclei acid molecule of (a), (b) or (c); (f) a polypeptide comprising an amino acid sequence with at least 95% sequence identity to a polypeptide encoded by a gene product in Table 2a or 2b; or (g) a polypeptide comprising an amino acid sequence encoded by a nucleic acid molecule having at least 95% sequence identity to a nucleic acid molecule consisting essentially of a nucleic acid sequence of a gene product of Table 2a.

In another aspect, the invention concerns a method of treating an individual with colon cancer, comprising the step of administering a composition containing: (a) a nucleic acid molecule consisting essentially of a nucleic acid sequence that encodes an amino acid sequence of a polypeptide encoded by a gene product in Table 2a; (b) a nucleic acid molecule consisting essentially of a nucleic acid sequence of a gene product in Table 2a; (c) a nucleic acid molecule consisting essentially of a nucleic acid sequence of Table 7; (d) a nucleic acid molecule that selectively hybridizes to the nucleic acid molecule of (a), (b) or (c); (e) a nucleic acid molecule having at least 95% sequence identity to the nucleic acid molecule of (a), (b) or (c); (f) a polypeptide comprising an amino acid sequence with at least 95% sequence identity to a polypeptide encoded by a gene product in Table 2a; (g) a polypeptide comprising an amino acid sequence encoded by a nucleic acid molecule having at least 95% sequence identity to a nucleic acid molecule comprising a nucleic acid sequence of a gene product of Table 2a; or (h) an appropriate agonist or antagonist for a gene product of Table 2a, to an individual in need thereof, wherein said administration induces an immune response against the colon cancer cell expressing the nucleic acid molecule or polypeptide.

### DETAILED DESCRIPTION OF THE INVENTION

### Definitions and General Techniques

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the instant specification unless otherwise indicated. See, e.g., Sambrook et al., Molecular Cloning: A Laboratory Manual, 2d ed., Cold Spring Harbor Laboratory Press (1989) and Sambrook et al., Molecular Cloning: A Laboratory Manual, 3d ed., Cold Spring Harbor Press (2001); Ausubel et al., Current Protocols in Molecular Biology, Greene Publishing Associates (1992, and Supplements to 2000); Ausubel et al., Short Protocols in Molecular Biology: A Compendium of Methods from Current Protocols in Molecular Biology-4^{th} Ed., Wiley & Sons (1999); Harlow and Lane, Antibodies: A Laboratory Manual, Cold Spring Harbor Laboratory Press (1990); and Harlow and Lane, Using Antibodies: A Laboratory Manual, Cold Spring Harbor Laboratory Press (1999).

Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, delivery and/or treatment of patients.

The following terms, unless otherwise indicated, shall be understood to have the following meanings:

A “nucleic acid molecule” of this invention refers to a polymeric form of nucleotides and includes both sense and antisense strands of RNA, cDNA, genomic DNA, and synthetic forms and mixed polymers of the above. A nucleotide refers to a ribonucleotide, deoxynucleotide or a modified form of either type of nucleotide. A “nucleic acid molecule” as used herein is synonymous with “nucleic acid” and “polynucleotide.” The term “nucleic acid molecule” usually refers to a molecule of at least 10 bases in length, unless otherwise specified. The term includes single and double stranded forms of DNA. In addition, a polynucleotide may include either or both naturally occurring and modified nucleotides linked together by naturally occurring and/or non-naturally occurring nucleotide linkages.

Nucleotides are represented by single letter symbols in nucleic acid molecule sequences. The following table lists symbols identifying nucleotides or groups of nucleotides which may occupy the symbol position on a nucleic acid molecule. See Nomenclature Committee of the International Union of Biochemistry (NC-IUB), Nomenclature for incompletely specified bases in nucleic acid sequences, Recommendations 1984., Eur J Biochem. 150(1):1-5 (1985).

| | | | Complementary |
|---|---|---|---|
| Symbol | Meaning | Group/Origin of Designation | Symbol |
| a | a | Adenine | t/u |
| g | g | Guanine | c |
| c | c | Cytosine | g |
| t | t | Thymine | a |
| u | u | Uracil | a |
| r | g or a | puRine | y |
| y | t/u or c | pYrimidine | r |
| m | a or c | aMino | k |
| k | g or t/u | Keto | m |
| s | g or c | Strong interactions 3H-bonds | w |
| w | a or t/u | Weak interactions 2H-bonds | s |
| b | g or c or t/u | not a | v |
| d | a or g or t/u | not c | h |
| h | a or c or t/u | not g | d |
| v | a or g or c | not t, not u | b |
| n | a or g or c | aNy | n |
| | or t/u, | | |
| | unknown, or | | |
| | other | | |

The nucleic acid molecules may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.) The term “nucleic acid molecule” also includes any topological conformation, including single-stranded, double-stranded, partially duplexed, triplexed, hairpinned, circular and padlocked conformations. Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

A “gene” is defined as a nucleic acid molecule that comprises a nucleic acid sequence that encodes a polypeptide and the expression control sequences that surround the nucleic acid sequence that encodes the polypeptide. For instance, a gene may comprise a promoter, one or more enhancers, a nucleic acid sequence that encodes a polypeptide, downstream regulatory sequences and, possibly, other nucleic acid sequences involved in regulation of the expression of an RNA. As is well known in the art, eukaryotic genes usually contain both exons and introns. The term “exon” refers to a nucleic acid sequence found in genomic DNA that is bioinformatically predicted and/or experimentally confirmed to contribute contiguous sequence to a mature mRNA transcript. The term “intron” refers to a nucleic acid sequence found in genomic DNA that is predicted and/or confirmed to not contribute to a mature mRNA transcript, but rather to be “spliced out” during processing of the transcript.

A “gene product” is defined as a molecule expressed or encoded directly or indirectly by a gene. For example, gene products include pre-mRNA, mature mRNA, tRNA, rRNA, snRNA, u1RNA, pre-polypeptides, pro-polypeptides, mature polypeptides, post translationally modified polypeptides, processed polypeptides, functionally active polypeptides, functionally inactive polypeptides, complexed polypeptides and naturally allelic variants thereof such as single nucleotide polymorphism (SNP) variants. A single gene product may have several molecular functions and different gene products may share a single or similar molecular function. A gene product may be referred to by the accession number or common abbreviated name of the gene which expresses or encodes the gene product.

The term “level(s) of gene product” is defined as a quantifiable measurement of the gene product. The measurement may be an assay to determine the amount or mass of the product in a sample, the amount of chemically or enzymatically active product in a sample, or the amount of biologically functional product in a sample. Examples of these assays include determining relative and total RNA expression, gene copies, pre-mRNA and mature mRNA levels, knockdown levels, regulatory or surrogate marker levels, ISH, FISH, immunoassays, IHC, proteomic assays and other assays described below.

The term “activity” of a gene product is defined as the biochemical or biological function of the gene product. Examples of gene product activities are listed in Table 1 below. Specific activities of gene products of the instant invention are disclosed in Gene Ontology databases or published literature and summarized in Table 3 below.

A nucleic acid molecule or polypeptide is “derived” from a particular species if the nucleic acid molecule or polypeptide has been isolated from the particular species, or if the nucleic acid molecule or polypeptide is homologous to a nucleic acid molecule or polypeptide isolated from a particular species.

An “isolated” or “substantially pure” nucleic acid or polynucleotide (e.g., an RNA, DNA or a mixed polymer) is one which is substantially separated from other cellular components that naturally accompany the native polynucleotide in its natural host cell, e.g., ribosomes, polymerases, or genomic sequences with which it is naturally associated. The term embraces a nucleic acid or polynucleotide that (1) has been removed from its naturally occurring environment, (2) is not associated with all or a portion of a polynucleotide in which the “isolated polynucleotide” is found in nature, (3) is operatively linked to a polynucleotide which it is not linked to in nature, (4) does not occur in nature as part of a larger sequence or (5) includes nucleotides or internucleoside bonds that are not found in nature. The term “isolated” or “substantially pure” also can be used in reference to recombinant or cloned DNA isolates, chemically synthesized polynucleotide analogs, or polynucleotide analogs that are biologically synthesized by heterologous systems. The term “isolated nucleic acid molecule” includes nucleic acid molecules that are integrated into a host cell chromosome at a heterologous site, recombinant fusions of a native fragment to a heterologous sequence, recombinant vectors present as episomes or as integrated into a host cell chromosome.

A “part” of a nucleic acid molecule refers to a nucleic acid molecule that comprises a partial contiguous sequence of at least 10 bases of the reference nucleic acid molecule and can range in length from at least 10 bases up to the full length reference nucleic acid sequence minus one nucleotide base. Thus, for example, when the full length reference nucleic acid molecule contains 1000 nucleotide bases, the part may contain from at least 10 up to 999 nucleotide bases of that reference nucleic acid molecule. Preferably, a part comprises at least 15 to 20 bases of a reference nucleic acid molecule. In theory, a nucleic acid sequence of 17 nucleotides is of sufficient length to occur at random less frequently than once in the three gigabase human genome, and thus to provide a nucleic acid probe that can uniquely identify the reference sequence in a nucleic acid mixture of genomic complexity. A preferred part is thus one which comprises at least 17 nucleotides and provides a nucleic acid probe specific for a reference nucleic acid molecule of the present invention. Another preferred part is one comprising a nucleic acid sequence, the expression of which is indicative of colon cancer. Another preferred part is one that comprises a nucleic acid sequence that can encode at least 6 contiguous amino acid sequences (fragments of at least 18 nucleotides) because they are useful in directing the expression or synthesis of peptides that are useful in mapping the epitopes of the polypeptide encoded by the reference nucleic acid. See, e.g., Geysen et al., Proc. Natl. Acad. Sci. USA 81:3998-4002 (1984); and U.S. Pat. Nos. 4,708,871 and 5,595,915, the disclosures of which are incorporated herein by reference in their entireties. Preferably the 6 contiguous amino acids comprise a contiguous region of amino acids identical to a portion of a cancer specific polypeptide (CaSP) of the present invention. A part may also comprise at least 25, 30, 35 or 40 nucleotides of a reference nucleic acid molecule, or at least 50, 60, 70, 80, 90, 100, 150, 200, 250, 300, 350, 400 or 500 nucleotides of a reference nucleic acid molecule. A part of a nucleic acid molecule may comprise no other nucleic acid sequences. Alternatively, a part of a nucleic acid may comprise other nucleic acid sequences from other nucleic acid molecules.

The term “oligonucleotide” refers to a nucleic acid molecule generally comprising a length of 200 bases or fewer. A nucleoside, as known by those skilled in the art, is a base-sugar combination. The base portion of a nucleoside is typically a heterocyclic base, the two most common classes of which are purines and the pyrimidines. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. In forming oligonucleotides, the phosphate groups covalently link adjacent nucleosides to one another to form a linear polymeric compound. In some embodiments, the respective ends of this linear polymeric structure can be further joined to form a circular structure. Within the oligonucleotide structure, the phosphate groups are commonly referred to as forming the internucleoside backbone of the oligonucleotide. The normal linkage or backbone of RNA and DNA is a 3′ to 5′ phosphodiester linkage. The term “oligonucleotide” often refers to single-stranded deoxyribonucleotides, but it can refer as well to single- or double-stranded ribonucleotides, RNA:DNA hybrids and double-stranded DNAs, among others.

Preferably, oligonucleotides are 10 to 60 bases in length and most preferably 12, 13, 14, 15, 16, 17, 18, 19 or 20 bases in length. Other preferred oligonucleotides are 25, 30, 35, 40, 45, 50, 55 or 60 bases in length. Oligonucleotides may be single-stranded, e.g. for use as probes or primers.

Thus, in the context of the present invention, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for a reference nucleic acid molecule and increased stability in the presence of nucleases.

Oligonucleotides, such as single-stranded DNA probe oligonucleotides, often are synthesized by chemical methods, such as those implemented on automated oligonucleotide synthesizers. However, oligonucleotides can be made by a variety of other methods, including in vitro recombinant DNA-mediated techniques and by expression of DNAs in cells and organisms. Initially, chemically synthesized DNAs typically are obtained without a 5′ phosphate. The 5′ ends of such oligonucleotides are not substrates for phosphodiester bond formation by ligation reactions that employ DNA ligases typically used to form recombinant DNA molecules. Where ligation of such oligonucleotides is desired, a phosphate can be added by standard techniques, such as those that employ a kinase and ATP. The 3′ end of a chemically synthesized oligonucleotide generally has a free hydroxyl group and, in the presence of a ligase, such as T4 DNA ligase, readily will form a phosphodiester bond with a 5′ phosphate of another polynucleotide, such as another oligonucleotide. As is well known, this reaction can be prevented selectively, where desired, by removing the 5′ phosphates of the other polynucleotide(s) prior to ligation.

Oligonucleotides of the present invention may further include ribozymes, external guide sequence (EGS), oligozymes, and other short catalytic RNAs or catalytic oligonucleotides which hybridize to the reference nucleic acid molecules.

The term “naturally occurring nucleotide” referred to herein includes naturally occurring deoxyribonucleotides and ribonucleotides. The term “modified nucleotides” referred to herein includes nucleotides with modified or substituted sugar groups and the like. The term “nucleotide linkages” referred to herein includes nucleotides linkages such as phosphorothioate, phosphorodithioate, phosphoroselenoate, phosphorodiselenoate, phosphoroanilothioate, phoshoraniladate, phosphoroamidate, and the like. See e.g., LaPlanche et al, Nucl. Acids Res. 14:9081-9093 (1986); Stein et al., Nucl. Acids Res. 16:3209-3221 (1988); Zon et al., Anti-Cancer Drug Design 6:539-568 (1991); Zon et al, in Eckstein (ed.) Oligonucleotides and Analogues: A Practical Approach, pp. 87-108, Oxford University Press (1991); Uhlmann and Peyman, Chemical Reviews 90:543 (1990), and U.S. Pat. No. 5,151,510, the disclosure of which is hereby incorporated by reference in its entirety.

Unless specified otherwise, the left hand end of a polynucleotide sequence in sense orientation is the 5′ end and the right hand end of the sequence is the 3′ end. In addition, the left hand direction of a polynucleotide sequence in sense orientation is referred to as the 5′ direction, while the right hand direction of the polynucleotide sequence is referred to as the 3′ direction. Further, unless otherwise indicated, each nucleotide sequence is set forth herein as a sequence of deoxyribonucleotides. It is intended, however, that the given sequence be interpreted as would be appropriate to the polynucleotide composition: for example, if the isolated nucleic acid is composed of RNA, the given sequence intends ribonucleotides, with uridine substituted for thymidine.

The term “allelic variant” refers to one of two or more alternative naturally occurring forms of a gene, wherein each gene possesses a unique nucleotide sequence. In a preferred embodiment, different alleles of a given gene have similar or identical biological properties.

The term “percent sequence identity” in the context of nucleic acid sequences refers to the residues in two sequences which are the same when aligned for maximum correspondence. The length of sequence identity comparison may be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA, which includes, e.g., the programs FASTA2 and FASTA3, provides alignments and percent sequence identity of the regions of the best overlap between the query and search sequences (Pearson, Methods Enzymol. 183: 63-98 (1990); Pearson, Methods Mol. Biol. 132: 185-219 (2000); Pearson, Methods Enzymol. 266: 227-258 (1996); Pearson, J. Mol. Biol. 276: 71-84 (1998)). Unless otherwise specified, default parameters for a particular program or algorithm are used. For instance, percent sequence identity between nucleic acid sequences can be determined using FASTA with its default parameters (a word size of 6 and the NOPAM factor for the scoring matrix) or using Gap with its default parameters as provided in GCG Version 6.1.

A reference to a nucleic acid sequence encompasses its complement unless otherwise specified. Thus, a reference to a nucleic acid molecule having a particular sequence should be understood to encompass its complementary strand, with its complementary sequence. The complementary strand is also useful, e.g., for antisense therapy, double stranded RNA (dsRNA) inhibition (RNAi), combination of triplex and antisense, hybridization probes and PCR primers.

In the molecular biology art, researchers use the terms “percent sequence identity”, “percent sequence similarity” and “percent sequence homology” interchangeably. In this application, these terms shall have the same meaning with respect to nucleic acid sequences only.

The term “substantial similarity” or “substantial sequence similarity,” when referring to a nucleic acid or fragment thereof, indicates that, when optimally aligned with appropriate nucleotide insertions or deletions with another nucleic acid (or its complementary strand), there is nucleotide sequence identity in at least about 50%, more preferably 60% of the nucleotide bases, usually at least about 70%, more usually at least about 80%, preferably at least about 90%, more preferably at least about 95-99%, and most preferably at least about 99.5-99.9% of the nucleotide bases, as measured by any well known algorithm of sequence identity, such as FASTA, BLAST or Gap, as discussed above.

Alternatively, substantial similarity exists between a first and second nucleic acid sequence when the first nucleic acid sequence or fragment thereof hybridizes to an antisense strand of the second nucleic acid, under selective hybridization conditions. Typically, selective hybridization will occur between the first nucleic acid sequence and an antisense strand of the second nucleic acid sequence when there is at least about 55% sequence identity between the first and second nucleic acid sequences, preferably at least about 65%, more preferably at least about 75%, more preferably at least about 90%, even more preferably at least about 95%, further preferably at least about 98%, and most preferably at least about 99%, 99.5%, 99.8% or 99.9%, over a stretch of at least about 14 nucleotides, more preferably at least 17 nucleotides, even more preferably at least 20, 25, 30, 35, 40, 50, 60, 70, 80, 90 or 100 nucleotides.

Alternatively, substantial similarity exists between a first and second nucleic acid sequence when the second nucleic acid sequence or fragment thereof hybridizes to an antisense strand of the first nucleic acid. Preferably, there is at least about 70% sequence identity between the first and second nucleic acid sequences, more preferably at least about 80%, more preferably at least about 90%, even more preferably at least about 95%, further preferably at least about 98%, and most preferably at least about 99%, 99.5%, 99.8% or 99.9% sequence identity, over the entire length of the second nucleic acid.

Nucleic acid hybridization will be affected by such conditions as salt concentration, temperature, solvents, the base composition of the hybridizing species, length of the complementary regions, and the number of nucleotide base mismatches between the hybridizing nucleic acids, as will be readily appreciated by those skilled in the art. “Stringent hybridization conditions” and “stringent wash conditions” in the context of nucleic acid hybridization experiments depend upon a number of different physical parameters. The most important parameters include temperature of hybridization, base composition of the nucleic acids, salt concentration and length of the nucleic acid. One having ordinary skill in the art knows how to vary these parameters to achieve a particular stringency of hybridization. In general, Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

In general “stringent hybridization” is performed at about 25° C. below the thermal melting point (Tₘ) for the specific DNA hybrid under a particular set of conditions. “Stringent washing” is performed at temperatures about 5° C. lower than the Tₘ for the specific DNA hybrid under a particular set of conditions. The Tₘ is the temperature at which 50% of the target sequence hybridizes to a perfectly matched probe. See Sambrook (1989), supra, p. 9.51.

The Tₘ for a particular DNA-DNA hybrid can be estimated by the formula:

Tₘ=81.5° C.+16.6(log₁₀[Na⁺]+0.41(fraction G+C)−0.63(% formamide)−(600/l) where l is the length of the hybrid in base pairs.

The Tₘ for a particular RNA-RNA hybrid can be estimated by the formula:

Tₘ=79.8° C.+18.5(log₁₀[Na⁺])+0.58(fraction G+C)+11.8(fraction G+C)²−0.35(% formamide)−(820/l).

The Tₘ for a particular RNA-DNA hybrid can be estimated by the formula:

Tₘ=79.8° C.+18.5(log₁₀[Na⁺])+0.58(fraction G+C)+11.8(fraction G+C)₂−0.50(% formamide)−(820/l).

In general, the Tₘ decreases by 1-1.5° C. for each 1% of mismatch between two nucleic acid sequences. Thus, one having ordinary skill in the art can alter hybridization and/or washing conditions to obtain sequences that have higher or lower degrees of sequence identity to the target nucleic acid. For instance, to obtain hybridizing nucleic acids that contain up to 10% mismatch from the target nucleic acid sequence, 10-15° C. would be subtracted from the calculated Tₘ of a perfectly matched hybrid, and then the hybridization and washing temperatures adjusted accordingly. Probe sequences may also hybridize specifically to duplex DNA under certain conditions to form triplex or other higher order DNA complexes. The preparation of such probes and suitable hybridization conditions are well known in the art.

Hybridization conditions for nucleic acid molecules that are shorter than 100 nucleotides in length (e.g., for oligonucleotide probes) may be calculated by the formula:

Tₘ=81.5° C.+16.6(log₁₀[Na⁺])+0.41(fraction G+C)−(600/N)

wherein N is change length and the [Na⁺] is 1 M or less. See Sambrook (1989), supra, p. 11.46. For hybridization of probes shorter than 100 nucleotides, hybridization is usually performed under stringent conditions (5-10° C. below the Tₘ) using high concentrations (0.1-1.0 pmol/ml) of probe. Id. at p. 11.45.

An example of “Stringent conditions” or “high stringency conditions”, as defined herein, typically: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5.times.SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 ug/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

Oligonucleotides utilized in PCR reactions (such as primers or probes) that hybridize to target nucleic acid gene products have a preferred Tₘ between 56° C. and 62° C. or more preferably between 58° C. and 60° C.

Determination of hybridization using mismatched probes, pools of degenerate probes or “guessmers,” as well as hybridization solutions and methods for empirically determining hybridization conditions are well known in the art. See, e.g., Ausubel (1999), supra; Sambrook (1989), supra, pp. 11.45-11.57.

The term “digestion” or “digestion of DNA” refers to catalytic cleavage of the DNA with a restriction enzyme that acts only at certain sequences in the DNA. The various restriction enzymes referred to herein are commercially available and their reaction conditions, cofactors and other requirements for use are known and routine to the skilled artisan. For analytical purposes, typically, 1 μg of plasmid or DNA fragment is digested with about 2 units of enzyme in about 20 μl of reaction buffer. For the purpose of isolating DNA fragments for plasmid construction, typically 5 to 50 μg of DNA are digested with 20 to 250 units of enzyme in proportionately larger volumes. Appropriate buffers and substrate amounts for particular restriction enzymes are described in standard laboratory manuals, such as those referenced below, and are specified by commercial suppliers. Incubation times of about 1 hour at 37° C. are ordinarily used, but conditions may vary in accordance with standard procedures, the supplier's instructions and the particulars of the reaction. After digestion, reactions may be analyzed, and fragments may be purified by electrophoresis through an agarose or polyacrylamide gel, using well known methods that are routine for those skilled in the art.

The term “ligation” refers to the process of forming phosphodiester bonds between two or more polynucleotides, which most often are double-stranded DNAs. Techniques for ligation are well known to the art and protocols for ligation are described in standard laboratory manuals and references, such as, e.g., Sambrook (1989), supra.

In one embodiment, the term “microarray” refers to a “nucleic acid microarray” having a substrate-bound plurality of nucleic acids, hybridization to each of the plurality of bound nucleic acids being separately detectable. The substrate can be solid or porous, planar or non-planar, unitary or distributed. Nucleic acid microarrays include all the devices so called in Schena (ed.), DNA Microarrays: A Practical Approach (Practical Approach Series), Oxford University Press (1999); Nature Genet. 21(1) (suppl.):1-60 (1999); Schena (ed.), Microarray Biochip: Tools and Technology, Eaton Publishing Company/BioTechniques Books Division (2000). Additionally, these nucleic acid microarrays include substrate-bound plurality of nucleic acids in which the plurality of nucleic acids are disposed on a plurality of beads, rather than on a unitary planar substrate, as is described, inter alia, in Brenner et al., Proc. Natl. Acad. Sci. USA 97(4):1665-1670 (2000). Examples of nucleic acid microarrays may be found in U.S. Pat. Nos. 6,391,623, 6,383,754, 6,383,749, 6,380,377, 6,379,897, 6,376,191, 6,372,431, 6,351,712 6,344,316, 6,316,193, 6,312,906, 6,309,828, 6,309,824, 6,306,643, 6,300,063, 6,287,850, 6,284,497, 6,284,465, 6,280,954, 6,262,216, 6,251,601, 6,245,518, 6,263,287, 6,251,601, 6,238,866, 6,228,575, 6,214,587, 6,203,989, 6,171,797, 6,103,474, 6,083,726, 6,054,274, 6,040,138, 6,083,726, 6,004,755, 6,001,309, 5,958,342, 5,952,180, 5,936,731, 5,843,655, 5,814,454, 5,837,196, 5,436,327, 5,412,087, 5,405,783, the disclosures of which are incorporated herein by reference in their entireties.

In an alternative embodiment, a “microarray” may also refer to a “peptide microarray” or “protein microarray” having a substrate-bound collection of plurality of polypeptides, the binding to each of the plurality of bound polypeptides being separately detectable. Alternatively, the peptide microarray may have a plurality of binders, including but not limited to monoclonal antibodies, polyclonal antibodies, phage display binders, yeast 2 hybrid binders, aptamers, which can specifically detect the binding of the polypeptides of this invention. The array may be based on autoantibody detection to the polypeptides of this invention, see Robinson et al., Nature Medicine 8(3):295-301 (2002). Examples of peptide arrays may be found in WO 02/31463, WO 02/25288, WO 01/94946, WO 01/88162, WO 01/68671, WO 01/57259, WO 00/61806, WO 00/54046, WO 00/47774, WO 99/40434, WO 99/39210, WO 97/42507 and U.S. Pat. Nos. 6,268,210, 5,766,960, 5,143,854, the disclosures of which are incorporated herein by reference in their entireties.

In addition, determination of the levels of the CaSNA or CaSP may be made in a multiplex manner using techniques described in WO 02/29109, WO 02/24959, WO 01/83502, WO01/73113, WO 01/59432, WO 01/57269, WO 99/67641, the disclosures of which are incorporated herein by reference in their entireties.

The term “recombinant host cell” (or simply “host cell”), as used herein, is intended to refer to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

As used herein, the phrase “open reading frame” and the equivalent acronym “ORF” refers to that portion of a transcript-derived nucleic acid that can be translated in its entirety into a sequence of contiguous amino acids. As so defined, an ORF has length, measured in nucleotides, exactly divisible by 3. As so defined, an ORF need not encode the entirety of a natural protein.

As used herein, the phrase “ORF-encoded peptide” refers to the predicted or actual translation of an ORF.

The term “polypeptide” encompasses both naturally occurring and non-naturally occurring proteins and polypeptides, as well as polypeptide fragments and polypeptide mutants, derivatives and analogs thereof. A polypeptide may be monomeric or polymeric. Further, a polypeptide may comprise a number of different modules within a single polypeptide each of which has one or more distinct activities. A preferred polypeptide in accordance with the invention comprises a CaSP encoded by a nucleic acid molecule of the instant invention, or a fragment, mutant, analog and derivative thereof.

The term “isolated protein” or “isolated polypeptide” is a protein or polypeptide that by virtue of its origin or source of derivation (1) is not associated with naturally associated components that accompany it in its native state, (2) is free of other proteins from the same species, (3) is expressed by a cell from a different species, or (4) does not occur in nature. Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well known in the art.

A protein or polypeptide is “substantially pure,” “substantially homogeneous” or “substantially purified” when at least about 60% to 75% of a sample exhibits a single species of polypeptide. The polypeptide or protein may be monomeric or multimeric. A substantially pure polypeptide or protein will typically comprise about 50%, 60%, 70%, 80% or 90% W/W of a protein sample, more usually about 95%, and preferably will be over 99% pure. Protein purity or homogeneity may be determined by a number of means well known in the art, such as polyacrylamide gel electrophoresis of a protein sample, followed by visualizing a single polypeptide band upon staining the gel with a stain well known in the art. For certain purposes, higher resolution may be provided by using HPLC or other means well known in the art for purification.

The term “fragment” when used herein with respect to polypeptides of the present invention refers to a polypeptide that has an amino-terminal and/or carboxy-terminal deletion compared to a full-length CaSP. In a preferred embodiment, the fragment is a contiguous sequence in which the amino acid sequence of the fragment is identical to the corresponding positions in the naturally occurring polypeptide. Fragments typically are at least 5, 6, 7, 8, 9 or 10 amino acids long, preferably at least 12, 14, 16 or 18 amino acids long, more preferably at least 20 amino acids long, more preferably at least 25, 30, 35, 40 or 45, amino acids, even more preferably at least 50 or 60 amino acids long, and even more preferably at least 70 amino acids long.

A “derivative” when used herein with respect to polypeptides of the present invention refers to a polypeptide which is substantially similar in primary structural sequence to a CaSP but which include, e.g., in vivo or in vitro chemical and biochemical modifications that are not found in the CaSP. Such modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

An “antibody” refers to an intact immunoglobulin, or to an antigen-binding portion thereof that competes with the intact antibody for specific binding to a molecular species, e.g., a polypeptide of the instant invention. Antigen-binding portions may be produced by recombinant DNA techniques or by enzymatic or chemical cleavage of intact antibodies. Antigen-binding portions include, inter alia, Fab, Fab′, F(ab′)₂, Fv, dAb, and complementarity determining region (CDR) fragments, single-chain antibodies (scFv), chimeric antibodies, diabodies and polypeptides that contain at least a portion of an immunoglobulin that is sufficient to confer specific antigen binding to the polypeptide. A Fab fragment is a monovalent fragment consisting of the VL, VH, CL and CH1 domains; a F(ab′)₂ fragment is a bivalent fragment comprising two Fab fragments linked by a disulfide bridge at the hinge region; a Fd fragment consists of the VH and CH1 domains; a Fv fragment consists of the VL and VH domains of a single arm of an antibody; and a dAb fragment consists of a VH domain. See, e.g., Ward et al., Nature 341: 544-546 (1989).

By “bind specifically” and “specific binding” as used herein it is meant the ability of the antibody to bind to a first molecular species in preference to binding to other molecular species with which the antibody and first molecular species are admixed. An antibody is said specifically to “recognize” a first molecular species when it can bind specifically to that first molecular species.

A single-chain antibody (scFv) is an antibody in which VL and VH regions are paired to form a monovalent molecule via a synthetic linker that enables them to be made as a single protein chain. See, e.g., Bird et al., Science 242: 423-426 (1988); Huston et al., Proc. Natl. Acad. Sci. USA 85: 5879-5883 (1988). Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites. See e.g., Holliger et al., Proc. Natl. Acad. Sci. USA 90: 6444-6448 (1993); Poljak et al., Structure 2: 1121-1123 (1994). One or more CDRs may be incorporated into a molecule either covalently or noncovalently to make it an immunoadhesin. An immunoadhesin may incorporate the CDR(s) as part of a larger polypeptide chain, may covalently link the CDR(s) to another polypeptide chain, or may incorporate the CDR(s) noncovalently. The CDRs permit the immunoadhesin to specifically bind to a particular antigen of interest. A chimeric antibody is an antibody that contains one or more regions from one antibody and one or more regions from one or more other antibodies.

An antibody may have one or more binding sites. If there is more than one binding site, the binding sites may be identical to one another or may be different. For instance, a naturally occurring immunoglobulin has two identical binding sites, a single-chain antibody or Fab fragment has one binding site, while a “bispecific” or “bifunctional” antibody has two different binding sites.

An “isolated antibody” is an antibody that (1) is not associated with naturally-associated components, including other naturally-associated antibodies, that accompany it in its native state, (2) is free of other proteins from the same species, (3) is expressed by a cell from a different species, or (4) does not occur in nature. It is known that purified proteins, including purified antibodies, may be stabilized with non-naturally-associated components. The non-naturally-associated component may be a protein, such as albumin (e.g., BSA) or a chemical such as polyethylene glycol (PEG).

The term “epitope” includes any protein determinant capable of specific binding to an immunoglobulin or T-cell receptor. Epitopic determinants usually consist of chemically active surface groupings of molecules such as amino acids or sugar side chains and usually have specific three-dimensional structural characteristics, as well as specific charge characteristics. An antibody is said to specifically bind an antigen when the dissociation constant is less than 1 μM, preferably less than 100 nM and most preferably less than 10 nM.

The terms “patient” and “individual” includes human and veterinary subjects.

Throughout this specification and claims, the word “comprise,” or variations such as “comprises” or “comprising,” will be understood to imply the inclusion of a stated integer or group of integers but not the exclusion of any other integer or group of integers.

The term “cancer specific,” for purposes of the present invention, refers to a nucleic acid molecule or polypeptide that is expressed predominantly in colon cancer as compared to other tissues in the body. In a preferred embodiment, a “cancer specific” nucleic acid molecule or polypeptide is detected at a level that is 1.5-fold higher than any other tissue in the body. In a more preferred embodiment, the “cancer specific” nucleic acid molecule or polypeptide is detected at a level that is 1.8-fold higher than any other tissue in the body, more preferably 2-fold higher, still more preferably at least 2.5-fold, 3-fold, 4-fold, 5-fold, 10-fold, 15-fold, 20-fold, 25-fold, 50-fold or 100-fold higher than any other tissue in the body.

In another preferred embodiment, a “cancer specific” nucleic acid molecule or polypeptide is detected at a level that is 1.5-fold lower than any other tissue in the body. In a more preferred embodiment, the “cancer specific” nucleic acid molecule or polypeptide is detected at a level that is 1.8-fold lower than any other tissue in the body, more preferably 2-fold lower, still more preferably at least 2.5-fold, 3-fold, 4-fold, 5-fold, 10-fold, 15-fold, 20-fold, 25-fold, 50-fold or 100-fold lower than any other tissue in the body.

Nucleic acid molecule levels may be measured by nucleic acid hybridization, such as Northern blot hybridization, microarray analysis or quantitative PCR. Polypeptide levels may be measured by any method known to accurately quantitate protein levels, such as Western blot analysis.

The term “prognosis” defines a forecast as to the probable outcome of a disease, the prospect as to recovery from a disease, or the potential recurrence of a disease as indicated by the nature and symptoms of the case. In general, prognosis is defined as “good” when there is a probable favorable outcome of a disease, recovery from a disease or low potential for disease recurrence. A “poor” prognosis is generally defined as a non-favorable outcome of a disease, non-recovery from a disease, or greater potential for disease recurrence. Prognosis may be determined using clinical factors, pathological evaluation, genotypic or phenotypic molecular profiling.

Nucleic acid molecules of the present invention are also inclusive of nucleic acid sequences containing modifications of the native nucleic acid molecule. Examples of such modifications include, but are not limited to, normative internucleoside bonds, post-synthetic modifications and altered nucleotide analogues. One having ordinary skill in the art would recognize that the type of modification that may be made will depend upon the intended use of the nucleic acid molecule. For instance, when the nucleic acid molecule is used as a hybridization probe, the range of such modifications will be limited to those that permit sequence-discriminating base pairing of the resulting nucleic acid. When used to direct expression of RNA or protein in vitro or in vivo, the range of such modifications will be limited to those that permit the nucleic acid to function properly as a polymerization substrate. When the isolated nucleic acid is used as a therapeutic agent, the modifications will be limited to those that do not confer toxicity upon the isolated nucleic acid.

Accordingly, in one embodiment, a nucleic acid molecule may include nucleotide analogues that incorporate labels that are directly detectable, such as radiolabels or fluorophores, or nucleotide analogues that incorporate labels that can be visualized in a subsequent reaction, such as biotin or various haptens. The labeled nucleic acid molecules are particularly useful as hybridization probes.

Common radiolabeled analogues include, but are not limited to, those labeled with ³³P, ³²P, and ³⁵S, such as α-³²P-dATP, α-³²P-dCTP, α-³²P-dGTP, α-³²P-dTTP, α-³²P-3′dATP, α-³²P-ATP, α-³²P-CTP, α-³²P-GTP, α-³²P-UTP, α-³⁵S-dATP, γ-³⁵S-GTP, γ-³³P-dATP, and the like.

Commercially available fluorescent nucleotide analogues readily incorporated into the nucleic acids of the present invention include, but are not limited to, Cy3-dCTP, Cy3-dUTP, Cy5-dCTP, Cy3-dUTP (Amersham Biosciences, Piscataway, N.J., USA), fluorescein-12-dUTP, tetramethylrhodamine-6-dUTP, Texas Red®-5-dUTP, Cascade Blue®-7-dUTP, BODIPY® FL-14-dUTP, BODIPY® TMR-14-dUTP, BODIPY® TR-14-dUTP, Rhodamine Green™-5-dUTP, Oregon Green® 488-5-dUTP, Texas Red®-12-dUTP, BODIPY® 630/650-14-dUTP, BODIPY® 650/665-14-dUTP, Alexa Fluor® 488-5-dUTP, Alexa Fluor® 532-5-dUTP, Alexa Fluor® 568-5-dUTP, Alexa Fluor® 594-5-dUTP, Alexa Fluor® 546-14-dUTP, fluorescein-12-UTP, tetramethylrhodamine-6-UTP, Texas Red®-5-UTP, Cascade Blue®-7-UTP, BODIPY® FL-14-UTP, BODIPY® TMR-14-UTP, BODIPY® TR-14-UTP, Rhodamine Green™-5-UTP, Alexa Fluor® 488-5-UTP and Alexa Fluor® 546-14-UTP (Molecular Probes, Inc. Eugene, Oreg., USA). One may also custom synthesize nucleotides having other fluorophores. See Henegariu et al., Nature Biotechnol. 18: 345-348 (2000).

Haptens that are commonly conjugated to nucleotides for subsequent labeling include, but are not limited to, biotin (biotin-11-dUTP, Molecular Probes, Inc., Eugene, Oreg., USA; biotin-21-UTP, biotin-21-dUTP, Clontech Laboratories, Inc., Palo Alto, Calif., USA), digoxigenin (DIG-11-dUTP, alkali labile, DIG-11-UTP, Roche Diagnostics Corp., Indianapolis, Ind., USA), and dinitrophenyl (dinitrophenyl-1-dUTP, Molecular Probes, Inc., Eugene, Oreg., USA).

Nucleic acid molecules of the present invention can be labeled by incorporation of labeled nucleotide analogues into the nucleic acid. Such analogues can be incorporated by enzymatic polymerization, such as by nick translation, random priming, polymerase chain reaction (PCR), terminal transferase tailing, and end-filling of overhangs, for DNA molecules, and in vitro transcription driven, e.g., from phage promoters, such as T7, T3, and SP6, for RNA molecules. Commercial kits are readily available for each such labeling approach. Analogues can also be incorporated during automated solid phase chemical synthesis. Labels can also be incorporated after nucleic acid synthesis, with the 5′ phosphate and 3′ hydroxyl providing convenient sites for post-synthetic covalent attachment of detectable labels.

Other post-synthetic approaches also permit internal labeling of nucleic acids. For example, fluorophores can be attached using a cisplatin reagent that reacts with the N7 of guanine residues (and, to a lesser extent, adenine bases) in DNA, RNA, and Peptide Nucleic Acids (PNA) to provide a stable coordination complex between the nucleic acid and fluorophore label (Universal Linkage System) (available from Molecular Probes, Inc., Eugene, Oreg., USA and Amersham Pharmacia Biotech, Piscataway, N.J., USA); see Alers et al., Genes, Chromosomes & Cancer 25: 301-305 (1999); Jelsma et al., J. NIH Res. 5: 82 (1994); Van Belkum et al., BioTechniques 16: 148-153 (1994). Alternatively, nucleic acids can be labeled using a disulfide-containing linker (FastTag™ Reagent, Vector Laboratories, Inc., Burlingame, Calif., USA) that is photo- or thermally coupled to the target nucleic acid using aryl azide chemistry; after reduction, a free thiol is available for coupling to a hapten, fluorophore, sugar, affinity ligand, or other marker.

One or more independent or interacting labels can be incorporated into the nucleic acid molecules of the present invention. For example, both a fluorophore and a moiety that in proximity thereto acts to quench fluorescence can be included to report specific hybridization through release of fluorescence quenching or to report exonucleotidic excision. See, e.g., Tyagi et al., Nature Biotechnol. 14: 303-308 (1996); Tyagi et al., Nature Biotechnol. 16: 49-53 (1998); Sokol et al., Proc. Natl. Acad. Sci. USA 95: 11538-11543 (1998); Kostrikis et al., Science 279: 1228-1229 (1998); Marras et al., Genet. Anal. 14: 151-156 (1999); Holland et al., Proc. Natl. Acad. Sci. USA 88: 7276-7280 (1991); Heid et al., Genome Res. 6(10): 986-94 (1996); Kuimelis et al., Nucleic Acids Symp. Ser (37): 255-6 (1997); and U.S. Pat. Nos. 5,846,726, 5,925,517, 5,925,517, 5,723,591 and 5,538,848, the disclosures of which are incorporated herein by reference in their entireties.

Nucleic acid molecules of the present invention may also be modified by altering one or more native phosphodiester internucleoside bonds to more nuclease-resistant, internucleoside bonds. See Hartmann et al. (eds.), Manual of Antisense Methodology: Perspectives in Antisense Science, Kluwer Law International (1999); Stein et al. (eds.), Applied Antisense Oligonucleotide Technology, Wiley-Liss (1998); Chadwick et al. (eds.), Oligonucleotides as Therapeutic Agents—Symposium No. 209, John Wiley & Son Ltd (1997). Such altered internucleoside bonds are often desired for techniques or for targeted gene correction, Gamper et al., Nucl. Acids Res. 28(21): 4332-4339 (2000). For double stranded RNA inhibition which may utilize either natural ds RNA or ds RNA modified in its, sugar, phosphate or base, see Hannon, Nature 418(11): 244-251 (2002); Fire et al. in WO 99/32619; Tuschl et al. in US2002/0086356; Kruetzer et al. in WO 00/44895, the disclosures of which are incorporated herein by reference in their entirety. For circular antisense, see Kool in U.S. Pat. No. 5,426,180, the disclosure of which is incorporated herein by reference in its entirety.

Modified oligonucleotide backbones include, without limitation, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Representative U.S. patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, the disclosures of which are incorporated herein by reference in their entireties. In a preferred embodiment, the modified internucleoside linkages may be used for antisense techniques.

Other modified oligonucleotide backbones do not include a phosphorus atom, but have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative U.S. patents that teach the preparation of the above backbones include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437 and 5,677,439; the disclosures of which are incorporated herein by reference in their entireties.

In other preferred nucleic acid molecules, both the sugar and the internucleoside linkage are replaced with novel groups, such as peptide nucleic acids (PNA). In PNA compounds, the phosphodiester backbone of the nucleic acid is replaced with an amide-containing backbone, in particular by repeating N-(2-aminoethyl) glycine units linked by amide bonds. Nucleobases are bound directly or indirectly to aza nitrogen atoms of the amide portion of the backbone, typically by methylene carbonyl linkages. PNA can be synthesized using a modified peptide synthesis protocol. PNA oligomers can be synthesized by both Fmoc and tBoc methods. Representative U.S. patents that teach the preparation of PNA compounds include, but are not limited to, U.S. Pat. Nos. 5,539,082; 5,714,331; and 5,719,262, each of which is herein incorporated by reference in its entirety. Automated PNA synthesis is readily achievable on commercial synthesizers (see, e.g., “PNA User's Guide,” Rev. 2, February 1998, Perseptive Biosystems Part No. 60138, Applied Biosystems, Inc., Foster City, Calif.). PNA molecules are advantageous for a number of reasons. First, because the PNA backbone is uncharged, PNA/DNA and PNA/RNA duplexes have a higher thermal stability than is found in DNA/DNA and DNA/RNA duplexes. The Tm of a PNA/DNA or PNA/RNA duplex is generally 1° C. higher per base pair than the Tm of the corresponding DNA/DNA or DNA/RNA duplex (in 100 mM NaCl). Second, PNA molecules can also form stable PNA/DNA complexes at low ionic strength, under conditions in which DNA/DNA duplex formation does not occur. Third, PNA also demonstrates greater specificity in binding to complementary DNA because a PNA/DNA mismatch is more destabilizing than DNA/DNA mismatch. A single mismatch in mixed a PNA/DNA 15-mer lowers the Tm by 8-20° C. (15° C. on average). In the corresponding DNA/DNA duplexes, a single mismatch lowers the Tm by 4-16° C. (11° C. on average). Because PNA probes can be significantly shorter than DNA probes, their specificity is greater. Fourth, PNA oligomers are resistant to degradation by enzymes, and the lifetime of these compounds is extended both in vivo and in vitro because nucleases and proteases do not recognize the PNA polyamide backbone with nucleobase sidechains. See, e.g., Ray et al., FASEB J. 14(9): 1041-60 (2000); Nielsen et al, Pharmacol Toxicol. 86(1): 3-7 (2000); Larsen et al., Biochim Biophys Acta. 1489(1): 159-66 (1999); Nielsen, Curr. Opin. Struct. Biol. 9(3): 353-7 (1999), and Nielsen, Curr. Opin. Biotechnol. 10(1): 71-5 (1999).

Unless otherwise specified, nucleic acid molecules of the present invention can include any topological conformation appropriate to the desired use; the term thus explicitly comprehends, among others, single-stranded, double-stranded, triplexed, quadruplexed, partially double-stranded, partially-triplexed, partially-quadruplexed, branched, hairpinned, circular, and padlocked conformations. Padlock conformations and their utilities are further described in Banér et al., Curr. Opin. Biotechnol. 12: 11-15 (2001); Escude et al., Proc. Natl. Acad. Sci. USA 14: 96(19):10603-7 (1999); and Nilsson et al., Science 265(5181): 2085-8 (1994). Triplex and quadruplex conformations, and their utilities, are reviewed in Praseuth et al., Biochim. Biophys. Acta. 1489(1): 181-206 (1999); Fox, Curr. Med. Chem. 7(1): 17-37 (2000); Kochetkova et al., Methods Mol. Biol. 130: 189-201 (2000); Chan et al., J. Mol. Med. 75(4): 267-82 (1997); Rowley et al., Mol Med 5(10): 693-700 (1999); Kool, Annu Rev Biophys Biomol Struct. 25: 1-28 (1996).

SNP Polymorphisms

Commonly, sequence differences between individuals involve differences in single nucleotide positions (SNPs). SNPs may account for 90% of human DNA polymorphisms. Collins et al., 8 Genome Res. 1229-31 (1998). SNPs include single base pair positions in genomic DNA at which different sequence alternatives (alleles) exist in a population. In addition, the least frequent allele generally must occur at a frequency of 1% or greater. DNA sequence variants with a reasonably high population frequency are observed approximately every 1,000 nucleotide across the genome, with estimates as high as 1 SNP per 350 base pairs. Wang et al., 280 Science 1077-82 (1998); Harding et al, 60 Am. J. Human Genet. 772-89 (1997); Taillon-Miller et al., Genome Res. 8:748-54 (1998); Cargill et al., Nat. Genet. 22:231-38 (1999); and Semple et al., Bioinform. Disc. Note 16:735-38 (2000). The frequency of SNPs varies with the type and location of the change. In base substitutions, two-thirds of the substitutions involve the C-T and G-A type. This variation in frequency can be related to 5-methylcytosine deamination reactions that occur frequently, particularly at CpG dinucleotides. Regarding location, SNPs occur at a much higher frequency in non-coding regions than in coding regions. Information on over one million variable sequences is already publicly available via the Internet and more such markers are available from commercial providers of genetic information. Kwok and Gu, Med. Today 5:538-53 (1999).

Several definitions of SNPs exist. See, e.g., Brooks, 235 Gene 177-86 (1999). As used herein, the term “single nucleotide polymorphism” or “SNP” includes all single base variants, thus including nucleotide insertions and deletions in addition to single nucleotide substitutions. There are two types of nucleotide substitutions. A transition is the replacement of one purine by another purine or one pyrimidine by another pyrimidine. A transversion is the replacement of a purine for a pyrimidine, or vice versa.

Numerous methods exist for detecting SNPs within a nucleotide sequence. A review of many of these methods can be found in Landegren et al., 8 Genome Res. 769-76 (1998). For example, a SNP in a genomic sample can be detected by preparing a Reduced Complexity Genome (RCG) from the genomic sample, then analyzing the RCG for the presence or absence of a SNP. See, e.g., WO 00/18960. Multiple SNPs in a population of target polynucleotides in parallel can be detected using, for example, the methods of WO 00/50869. Other SNP detection methods include the methods of U.S. Pat. Nos. 6,297,018 and 6,322,980. Furthermore, SNPs can be detected by restriction fragment length polymorphism (RFLP) analysis. See, e.g., U.S. Pat. Nos. 5,324,631; 5,645,995. RFLP analysis of SNPs, however, is limited to cases where the SNP either creates or destroys a restriction enzyme cleavage site. SNPs can also be detected by direct sequencing of the nucleotide sequence of interest. In addition, numerous assays based on hybridization have also been developed to detect SNPs and mismatch distinction by polymerases and ligases. Several web sites provide information about SNPs including Ensembl (ensembl with the extension .org of the world wide web), Sanger Institute (sanger with the extension .ac.uk/genetics/exon/ of the world wide web), National Center for Biotechnology Information (NCBI) (ncbi with the extension .nlm.nih.gov/SNP/ of the world wide web), The SNP Consortium Ltd. (snp with the extension .cshl.org/ of the world wide web). In addition, one of ordinary skill in the art could perform a search against the genome or any of the databases cited above using BLAST to find the chromosomal location or locations of SNPs. Another a preferred method to find the genomic coordinates and associated SNPs would be to use the BLAT tool (genome with the extension .ucsc.edu of the world wide web, Kent et al. 2001, The Human Genome Browser at UCSC, Genome Research 996-1006 or Kent 2002 BLAT, The BLAST-Like Alignment Tool Genome Research, 1-9). All web sites above were accessed Dec. 3, 2003.

Methods for Using Nucleic Acid Molecules as Probes and Primers

The isolated nucleic acid molecules of the present invention can be used as hybridization probes to detect, characterize, and quantify hybridizing nucleic acids in, and isolate hybridizing nucleic acids from, both genomic and transcript-derived nucleic acid samples. When free in solution, such probes are typically, but not invariably, detectably labeled. When bound to a substrate, as in a microarray, such probes are typically, but not invariably unlabeled.

In one embodiment, the isolated nucleic acid molecules of the present invention can be used as probes to detect and characterize gross alterations in the gene of a CaSNA, such as a deletion, insertion, translocation, and/or duplication of the CaSNA genomic locus, through fluorescence in situ hybridization (FISH) to chromosome spreads. See, e.g., Andreeff et al (eds.), Introduction to Fluorescence In Situ Hybridization: Principles and Clinical Applications, John Wiley & Sons (1999). The isolated nucleic acid molecules of the present invention can be used as probes to assess smaller genomic alterations using, e.g., Southern blot detection of restriction fragment length polymorphisms. The isolated nucleic acid molecules of the present invention can be used as probes to isolate genomic clones that include a nucleic acid molecule of the present invention, which thereafter can be restriction mapped and sequenced to identify deletions, insertions, translocations, and substitutions (including single nucleotide polymorphisms, SNPs) at the sequence level. Alternatively, detection techniques such as molecular beacons may be used, see Kostrikis et al., Science 279:1228-1229 (1998).

The isolated nucleic acid molecules of the present invention can also be used as probes to detect, characterize, and quantify CaSNA in, and isolate CaSNA from, transcript-derived nucleic acid samples. In one embodiment, the isolated nucleic acid molecules of the present invention can be used as hybridization probes to detect, characterize by length, and quantify mRNA by Northern blot of total or poly-A⁺-selected RNA samples. In another embodiment, the isolated nucleic acid molecules of the present invention can be used as hybridization probes to detect, characterize by location, and quantify mRNA by in situ hybridization to tissue sections. See, e.g., Schwarchzacher et al., In Situ Hybridization, Springer-Verlag N.Y. (2000). In another preferred embodiment, the isolated nucleic acid molecules of the present invention can be used as hybridization probes to measure the representation of clones in a cDNA library or to isolate hybridizing nucleic acid molecules acids from cDNA libraries, permitting sequence level characterization of mRNAs that hybridize to CaSNAs, including, without limitations, identification of deletions, insertions, substitutions, truncations, alternatively spliced forms and single nucleotide polymorphisms. In yet another preferred embodiment, the nucleic acid molecules of the instant invention may be used in microarrays.

All of the aforementioned probe techniques are well within the skill in the art, and are described at greater length in standard texts such as Sambrook (2001), supra; Ausubel (1999), supra; and Walker et al. (eds.), The Nucleic Acids Protocols Handbook, Humana Press (2000).

In another embodiment, a nucleic acid molecule of the invention may be used as a probe or primer to identify and/or amplify a second nucleic acid molecule that selectively hybridizes to the nucleic acid molecule of the invention. In this embodiment, it is preferred that the probe or primer be derived from a nucleic acid molecule encoding a CaSP. More preferably, the probe or primer is derived from a nucleic acid molecule encoding a polypeptide having an amino acid sequence of a gene product of Table 2a or Table 2b. Also preferred are probes or primers derived from a CaSNA. More preferred are probes or primers derived from a nucleic acid molecule having a nucleotide sequence of a gene product of Table 2a, Table 2b or Table 7.

In general, a probe or primer is at least 10 nucleotides in length, more preferably at least 12, more preferably at least 14 and even more preferably at least 16 or 17 nucleotides in length. In an even more preferred embodiment, the probe or primer is at least 18 nucleotides in length, even more preferably at least 20 nucleotides and even more preferably at least 22 nucleotides in length. Primers and probes may also be longer in length. For instance, a probe or primer may be 25 nucleotides in length, or may be 30, 40 or 50 nucleotides in length. Methods of performing nucleic acid hybridization using oligonucleotide probes are well known in the art. See, e.g., Sambrook et al., 1989, supra, Chapter 11 and pp. 11.31-11.32 and 11.40-11.44, which describes radiolabeling of short probes, and pp. 11.45-11.53, which describe hybridization conditions for oligonucleotide probes, including specific conditions for probe hybridization (pp. 11.50-11.51).

Methods of performing primer-directed amplification are also well known in the art. Methods for performing the polymerase chain reaction (PCR) are compiled, inter alia, in McPherson, PCR Basics: From Background to Bench, Springer Verlag (2000); Innis et al. (eds.), PCR Applications: Protocols for Functional Genomics, Academic Press (1999); Gelfand et al. (eds.), PCR Strategies, Academic Press (1998); Newton et al., PCR, Springer-Verlag N.Y. (1997); Burke (ed.), PCR: Essential Techniques, John Wiley & Son Ltd (1996); White (ed.), PCR Cloning Protocols: From Molecular Cloning to Genetic Engineering, Vol. 67, Humana Press (1996); and McPherson et al. (eds.), PCR 2: A Practical Approach, Oxford University Press, Inc. (1995). Methods for performing RT-PCR are collected, e.g., in Siebert et al. (eds.), Gene Cloning and Analysis by RT-PCR, Eaton Publishing Company/Bio Techniques Books Division, 1998; and Siebert (ed.), PCR Technique: RT-PCR, Eaton Publishing Company/BioTechniques Books (1995).

PCR and hybridization methods may be used to identify and/or isolate nucleic acid molecules of the present invention including allelic variants, homologous nucleic acid molecules and fragments. PCR and hybridization methods may also be used to identify, amplify and/or isolate nucleic acid molecules of the present invention that encode homologous proteins, analogs, fusion protein or muteins of the invention. Nucleic acid primers as described herein can be used to prime amplification of nucleic acid molecules of the invention, using transcript-derived or genomic DNA as template.

These nucleic acid primers can also be used, for example, to prime single base extension (SBE) for SNP detection (See, e.g., U.S. Pat. No. 6,004,744, the disclosure of which is incorporated herein by reference in its entirety).

Isothermal amplification approaches, such as rolling circle amplification, are also now well-described. See, e.g., Schweitzer et al., Curr. Opin. Biotechnol. 12(1): 21-7 (2001); international patent publications WO 97/19193 and WO 00/15779, and U.S. Pat. Nos. 5,854,033 and 5,714,320, the disclosures of which are incorporated herein by reference in their entireties. Rolling circle amplification can be combined with other techniques to facilitate SNP detection. See, e.g., Lizardi et al., Nature Genet. 19(3): 225-32 (1998).

Nucleic acid molecules of the present invention may be bound to a substrate either covalently or noncovalently. The substrate can be porous or solid, planar or non-planar, unitary or distributed. The bound nucleic acid molecules may be used as hybridization probes, and may be labeled or unlabeled. In a preferred embodiment, the bound nucleic acid molecules are unlabeled.

In one embodiment, the nucleic acid molecule of the present invention is bound to a porous substrate, e.g., a membrane, typically comprising nitrocellulose, nylon, or positively charged derivatized nylon. The nucleic acid molecule of the present invention can be used to detect a hybridizing nucleic acid molecule that is present within a labeled nucleic acid sample, e.g., a sample of transcript-derived nucleic acids. In another embodiment, the nucleic acid molecule is bound to a solid substrate, including, without limitation, glass, amorphous silicon, crystalline silicon or plastics. Examples of plastics include, without limitation, polymethylacrylic, polyethylene, polypropylene, polyacrylate, polymethylmethacrylate, polyvinylchloride, polytetrafluoroethylene, polystyrene, polycarbonate, polyacetal, polysulfone, celluloseacetate, cellulosenitrate, nitrocellulose, or mixtures thereof. The solid substrate may be any shape, including rectangular, disk-like and spherical. In a preferred embodiment, the solid substrate is a microscope slide or slide-shaped substrate.

The nucleic acid molecule of the present invention can be attached covalently to a surface of the support substrate or applied to a derivatized surface in a chaotropic agent that facilitates denaturation and adherence by presumed noncovalent interactions, or some combination thereof. The nucleic acid molecule of the present invention can be bound to a substrate to which a plurality of other nucleic acids are concurrently bound, hybridization to each of the plurality of bound nucleic acids being separately detectable. At low density, e.g. on a porous membrane, these substrate-bound collections are typically denominated macroarrays; at higher density, typically on a solid support, such as glass, these substrate bound collections of plural nucleic acids are colloquially termed microarrays. As used herein, the term microarray includes arrays of all densities. It is, therefore, another aspect of the invention to provide microarrays that comprise one or more of the nucleic acid molecules of the present invention.

In yet another embodiment, the invention is directed to single exon probes based on the CaSNAs disclosed herein.

As further described below, the polypeptides of the present invention can readily be used as specific immunogens to raise antibodies that specifically recognize polypeptides of the present invention including CaSPs and their allelic variants and homologues. The antibodies, in turn, can be used, inter alia, specifically to assay for the polypeptides of the present invention, particularly CaSPs, e.g. by ELISA for detection of protein fluid samples, such as serum, by immunohistochemistry or laser scanning cytometry, for detection of protein in tissue samples, or by flow cytometry, for detection of intracellular protein in cell suspensions, for specific antibody-mediated isolation and/or purification of CaSPs, as for example by immunoprecipitation, and for use as specific agonists or antagonists of CaSPs.

### Antibodies

In another aspect, the invention provides antibodies, including fragments and derivatives thereof, which bind specifically to polypeptides encoded by the nucleic acid molecules of the present invention. In a preferred embodiment, the antibodies are specific for a polypeptide that is a CaSP, or a fragment, mutein, derivative, analog or fusion protein thereof. In a more preferred embodiment, the antibodies are specific for a polypeptide encoded by a gene product of Table 2a or Table 2b, or a fragment, mutein, derivative, analog or fusion protein thereof.

The antibodies of the present invention can be specific for linear epitopes, discontinuous epitopes, or conformational epitopes of such proteins or protein fragments, either as present on the protein in its native conformation or, in some cases, as present on the proteins as denatured, as, e.g., by solubilization in SDS. New epitopes may be also due to a difference in post translational modifications (PTMs) in disease versus normal tissue. For example, a particular site on a CaSP may be glycosylated in cancerous cells, but not glycosylated in normal cells or vice versa. In addition, alternative splice forms of a CaSP may be indicative of cancer. Differential degradation of the C or N-terminus of a CaSP may also be a marker or target for anticancer therapy. For example, a CaSP may be N-terminal degraded in cancer cells exposing new epitopes to which antibodies may selectively bind for diagnostic or therapeutic uses.

As is well known in the art, the degree to which an antibody can discriminate as among molecular species in a mixture will depend, in part, upon the conformational relatedness of the species in the mixture; typically, the antibodies of the present invention will discriminate over adventitious binding to non-CaSP polypeptides by at least two-fold, more typically by at least 5-fold, typically by more than 10-fold, 25-fold, 50-fold, 75-fold, and often by more than 100-fold, and on occasion by more than 500-fold or 1000-fold. When used to detect the proteins or protein fragments of the present invention, the antibody of the present invention is sufficiently specific when it can be used to determine the presence of the polypeptide of the present invention in samples derived from normal or cancerous human colon tissue.

Typically, the affinity or avidity of an antibody (or antibody multimer, as in the case of an IgM pentamer) of the present invention for a protein or protein fragment of the present invention will be at least about 1×10⁻⁶ molar (M), typically at least about 5×10⁻⁷ M, 1×10⁻⁷ M, with affinities and avidities of at least 1×10⁻⁸ M, 5×10⁻⁹ M, 1×10⁻¹⁰ M and up to 1×10⁻¹³ M proving especially useful.

The antibodies of the present invention can be naturally occurring forms, such as IgG, IgM, IgD, IgE, IgY, and IgA, from any avian, reptilian, or mammalian species.

Human antibodies can be drawn directly from human donors or human cells. In such case, antibodies to the polypeptides of the present invention will typically have resulted from fortuitous immunization, such as autoimmune immunization, with the polypeptide of the present invention. Such antibodies will typically, but will not invariably, be polyclonal. In addition, individual polyclonal antibodies may be isolated and cloned to generate monoclonals.

Human antibodies are more frequently obtained using transgenic animals that express human immunoglobulin genes, which transgenic animals can be affirmatively immunized with the protein immunogen of the present invention. Human Ig-transgenic mice capable of producing human antibodies and methods of producing human antibodies therefrom upon specific immunization are described, inter alia, in U.S. Pat. Nos. 6,162,963; 6,150,584; 6,114,598; 6,075,181; 5,939,598; 5,877,397; 5,874,299; 5,814,318; 5,789,650; 5,770,429; 5,661,016; 5,633,425; 5,625,126; 5,569,825; 5,545,807; 5,545,806, and 5,591,669, the disclosures of which are incorporated herein by reference in their entireties. Such antibodies are typically monoclonal, and are typically produced using techniques developed for production of murine antibodies.

Human antibodies are particularly useful, and often preferred, when the antibodies of the present invention are to be administered to human beings as in vivo diagnostic or therapeutic agents, since recipient immune response to the administered antibody will often be substantially less than that occasioned by administration of an antibody derived from another species, such as mouse.

IgG, IgM, IgD, IgE, IgY and IgA antibodies of the present invention are also usefully obtained from other species, including mammals such as rodents (typically mouse, but also rat, guinea pig, and hamster), lagomorphs (typically rabbits), and also larger mammals, such as sheep, goats, cows, and horses; or egg laying birds or reptiles such as chickens or alligators. In such cases, as with the transgenic human-antibody-producing non-human mammals, fortuitous immunization is not required, and the non-human mammal is typically affirmatively immunized, according to standard immunization protocols, with the polypeptide of the present invention. One form of avian antibodies may be generated using techniques described in WO 00/29444, published 25 May 2000.

As discussed above, virtually all fragments of 8 or more contiguous amino acids of a polypeptide of the present invention can be used effectively as immunogens when conjugated to a carrier, typically a protein such as bovine thyroglobulin, keyhole limpet hemocyanin, or bovine serum albumin, conveniently using a bifunctional linker such as those described elsewhere above, which discussion is incorporated by reference here.

Immunogenicity can also be conferred by fusion of the polypeptide of the present invention to other moieties. For example, polypeptides of the present invention can be produced by solid phase synthesis on a branched polylysine core matrix; these multiple antigenic peptides (MAPs) provide high purity, increased avidity, accurate chemical definition and improved safety in vaccine development. Tam et al., Proc. Natl. Acad. Sci. USA 85: 5409-5413 (1988); Posnett et al., J. Biol. Chem. 263: 1719-1725 (1988).

Protocols for immunizing non-human mammals or avian species are well-established in the art. See Harlow et al. (eds.), Using Antibodies: A Laboratory Manual, Cold Spring Harbor Laboratory (1998); Coligan et al. (eds.), Current Protocols in Immunology, John Wiley & Sons, Inc. (2001); Zola, Monoclonal Antibodies: Preparation and Use of Monoclonal Antibodies and Engineered Antibody Derivatives (Basics: From Background to Bench, Springer Verlag (2000); Gross M, Speck J. Dtsch. Tierarztl. Wochenschr. 103: 417-422 (1996). Immunization protocols often include multiple immunizations, either with or without adjuvants such as Freund's complete adjuvant and Freund's incomplete adjuvant, and may include naked DNA immunization (Moss, Semin. Immunol. 2: 317-327 (1990).

Antibodies from non-human mammals and avian species can be polyclonal or monoclonal, with polyclonal antibodies having certain advantages in immunohistochemical detection of the polypeptides of the present invention and monoclonal antibodies having advantages in identifying and distinguishing particular epitopes of the polypeptides of the present invention. Antibodies from avian species may have particular advantage in detection of the polypeptides of the present invention, in human serum or tissues (Vikinge et al., Biosens. Bioelectron. 13: 1257-1262 (1998). Following immunization, the antibodies of the present invention can be obtained using any art-accepted technique. Such techniques are well known in the art and are described in detail in references such as Coligan, supra; Zola, supra; Howard et al. (eds.), Basic Methods in Antibody Production and Characterization, CRC Press (2000); Harlow, supra; Davis (ed.), Monoclonal Antibody Protocols, Vol. 45, Humana Press (1995); Delves (ed.), Antibody Production Essential Techniques, John Wiley & Son Ltd (1997); and Kenney, Antibody Solution An Antibody Methods Manual, Chapman & Hall (1997).

Briefly, such techniques include, inter alia, production of monoclonal antibodies by hybridomas and expression of antibodies or fragments or derivatives thereof from host cells engineered to express immunoglobulin genes or fragments thereof. These two methods of production are not mutually exclusive: genes encoding antibodies specific for the polypeptides of the present invention can be cloned from hybridomas and thereafter expressed in other host cells. Nor need the two necessarily be performed together: e.g., genes encoding antibodies specific for the polypeptides of the present invention can be cloned directly from B cells known to be specific for the desired protein, as further described in U.S. Pat. No. 5,627,052, the disclosure of which is incorporated herein by reference in its entirety, or from antibody-displaying phage.

Recombinant expression in host cells is particularly useful when fragments or derivatives of the antibodies of the present invention are desired.

Host cells for recombinant antibody production of whole antibodies, antibody fragments, or antibody derivatives can be prokaryotic or eukaryotic.

Prokaryotic hosts are particularly useful for producing phage displayed antibodies of the present invention.

The technology of phage-displayed antibodies, in which antibody variable region fragments are fused, for example, to the gene III protein (pIII) or gene VIII protein (pVIII) for display on the surface of filamentous phage, such as M13, is by now well-established. See, e.g., Sidhu, Curr. Opin. Biotechnol. 11 (6): 610-6 (2000); Griffiths et al., Curr. Opin. Biotechnol. 9(1): 102-8 (1998); Hoogenboom et al., Immunotechnology, 4(1): 1-20 (1998); Rader et al., Current Opinion in Biotechnology 8: 503-508 (1997); Aujame et al., Human Antibodies 8: 155-168 (1997); Hoogenboom, Trends in Biotechnol. 15: 62-70 (1997); de Kruif et al., 17: 453-455 (1996); Barbas et al., Trends in Biotechnol. 14: 230-234 (1996); Winter et al., Ann. Rev. Immunol. 433-455 (1994). Techniques and protocols required to generate, propagate, screen (pan), and use the antibody fragments from such libraries have recently been compiled. See, e.g., Barbas (2001), supra; Kay, supra; and Abelson, supra.

Typically, phage-displayed antibody fragments are scFv fragments or Fab fragments; when desired, full length antibodies can be produced by cloning the variable regions from the displaying phage into a complete antibody and expressing the full length antibody in a further prokaryotic or a eukaryotic host cell. Eukaryotic cells are also useful for expression of the antibodies, antibody fragments, and antibody derivatives of the present invention. For example, antibody fragments of the present invention can be produced in Pichia pastoris and in Saccharomyces cerevisiae. See, e.g., Takahashi et al., Biosci. Biotechnol. Biochem. 64(10): 2138-44 (2000); Freyre et al., J. Biotechnol. 76(2-3):1 57-63 (2000); Fischer et al., Biotechnol. Appl Biochem. 30 (Pt 2): 117-20 (1999); Pennell et al., Res. Immunol. 149(6): 599-603 (1998); Eldin et al., J. Immunol. Methods. 201(1): 67-75 (1997); Frenken et al, Res. Immunol. 149(6): 589-99 (1998); and Shusta et al., Nature Biotechnol. 16(8): 773-7 (1998).

Antibodies, including antibody fragments and derivatives, of the present invention can also be produced in insect cells. See, e.g., Li et al., Protein Expr. Purif. 21(1): 121-8 (2001); Ailor et al., Biotechnol. Bioeng. 58(2-3): 196-203 (1998); Hsu et al., Biotechnol. Prog 13(1): 96-104 (1997); Edelman et al., Immunology 91(1): 13-9 (1997); and Nesbit et al., J. Immunol. Methods 151(1-2): 201-8 (1992).

Antibodies and fragments and derivatives thereof of the present invention can also be produced in plant cells, particularly maize or tobacco, Giddings et al., Nature Biotechnol. 18(11): 1151-5 (2000); Gavilondo et al., Biotechniques 29(1): 128-38 (2000); Fischer et al., J. Biol. Regul. Homeost. Agents 14(2): 83-92 (2000); Fischer et al., Biotechnol. Appl. Biochem. 30 (Pt 2): 113-6 (1999); Fischer et al., Biol. Chem. 380(7-8): 825-39 (1999); Russell, Curr. Top. Microbiol. Immunol. 240: 119-38 (1999); and Ma et al., Plant Physiol. 109(2): 341-6 (1995).

Antibodies, including antibody fragments and derivatives, of the present invention can also be produced in transgenic, non-human, mammalian milk. See, e.g. Pollock et al., J. Immunol Methods. 231: 147-57 (1999); Young et al., Res. Immunol. 149: 609-10 (1998); and Limonta et al., Immunotechnology 1: 107-13 (1995).

Mammalian cells useful for recombinant expression of antibodies, antibody fragments, and antibody derivatives of the present invention include CHO cells, COS cells, 293 cells, and myeloma cells. Verma et al., J. Immunol. Methods 216(1-2):165-81 (1998) review and compare bacterial, yeast, insect and mammalian expression systems for expression of antibodies. Antibodies of the present invention can also be prepared by cell free translation, as further described in Merk et al., J. Biochem. (Tokyo) 125(2): 328-33 (1999) and Ryabova et al., Nature Biotechnol 15(1): 79-84 (1997), and in the milk of transgenic animals, as further described in Pollock et al., J. Immunol. Methods 231(1-2): 147-57 (1999).

The invention further provides antibody fragments that bind specifically to one or more of the polypeptides of the present invention, to one or more of the polypeptides encoded by the isolated nucleic acid molecules of the present invention, or the binding of which can be competitively inhibited by one or more of the polypeptides of the present invention or one or more of the polypeptides encoded by the isolated nucleic acid molecules of the present invention. Among such useful fragments are Fab, Fab′, Fv, F(ab)′₂, and single chain Fv (scFv) fragments. Other useful fragments are described in Hudson, Curr. Opin. Biotechnol. 9(4): 395-402 (1998).

The present invention also relates to antibody derivatives that bind specifically to one or more of the polypeptides of the present invention, to one or more of the polypeptides encoded by the isolated nucleic acid molecules of the present invention, or the binding of which can be competitively inhibited by one or more of the polypeptides of the present invention or one or more of the polypeptides encoded by the isolated nucleic acid molecules of the present invention.

Among such useful derivatives are chimeric, primatized, and humanized antibodies; such derivatives are less immunogenic in human beings, and thus are more suitable for in vivo administration, than are unmodified antibodies from non-human mammalian species. Another useful method is PEGylation to increase the serum half life of the antibodies.

Chimeric antibodies typically include heavy and/or light chain variable regions (including both CDR and framework residues) of immunoglobulins of one species, typically mouse, fused to constant regions of another species, typically human. See, e.g., Morrison et al., Proc. Natl. Acad. Sci. USA. 81(21): 6851-5 (1984); Sharon et al., Nature 309(5966): 364-7 (1984); Takeda et al., Nature 314(6010): 452-4 (1985); and U.S. Pat. No. 5,807,715 the disclosure of which is incorporated herein by reference in its entirety. Primatized and humanized antibodies typically include heavy and/or light chain CDRs from a murine antibody grafted into a non-human primate or human antibody V region framework, usually further comprising a human constant region, Riechmann et al., Nature 332(6162): 323-7 (1988); Co et al., Nature 351(6326): 501-2 (1991); and U.S. Pat. Nos. 6,054,297; 5,821,337; 5,770,196; 5,766,886; 5,821,123; 5,869,619; 6,180,377; 6,013,256; 5,693,761; and 6,180,370, the disclosures of which are incorporated herein by reference in their entireties. Other useful antibody derivatives of the invention include heteromeric antibody complexes and antibody fusions, such as diabodies (bispecific antibodies), single-chain diabodies, and intrabodies.

It is contemplated that the nucleic acids encoding the antibodies of the present invention can be operably joined to other nucleic acids forming a recombinant vector for cloning or for expression of the antibodies of the invention. Accordingly, the present invention includes any recombinant vector containing the coding sequences, or part thereof, whether for eukaryotic transduction, transfection or gene therapy. Such vectors may be prepared using conventional molecular biology techniques, known to those with skill in the art, and would comprise DNA encoding sequences for the immunoglobulin V-regions including framework and CDRs or parts thereof, and a suitable promoter either with or without a signal sequence for intracellular transport. Such vectors may be transduced or transfected into eukaryotic cells or used for gene therapy (Marasco et al., Proc. Natl. Acad. Sci. (USA) 90: 7889-7893 (1993); Duan et al., Proc. Natl. Acad. Sci. (USA) 91: 5075-5079 (1994), by conventional techniques, known to those with skill in the art.

The antibodies of the present invention, including fragments and derivatives thereof, can usefully be labeled. It is, therefore, another aspect of the present invention to provide labeled antibodies that bind specifically to one or more of the polypeptides of the present invention, to one or more of the polypeptides encoded by the isolated nucleic acid molecules of the present invention, or the binding of which can be competitively inhibited by one or more of the polypeptides of the present invention or one or more of the polypeptides encoded by the isolated nucleic acid molecules of the present invention. The choice of label depends, in part, upon the desired use.

For example, when the antibodies of the present invention are used for immunohistochemical staining of tissue samples, the label can usefully be an enzyme that catalyzes production and local deposition of a detectable product. Enzymes typically conjugated to antibodies to permit their immunohistochemical visualization are well known, and include alkaline phosphatase, β-galactosidase, glucose oxidase, horseradish peroxidase (HRP), and urease. Typical substrates for production and deposition of visually detectable products include o-nitrophenyl-beta-D-galactopyranoside (ONPG); o-phenylenediamine dihydrochloride (OPD); p-nitrophenyl phosphate (PNPP); p-nitrophenyl-beta-D-galactopryanoside (PNPG); 3′,3′-diaminobenzidine (DAB); 3-amino-9-ethylcarbazole (AEC); 4-chloro-1-naphthol (CN); 5-bromo-4-chloro-3-indolyl-phosphate (BCIP); ABTS®; BluoGal; iodonitrotetrazolium (INT); nitroblue tetrazolium chloride (NBT); phenazine methosulfate (PMS); phenolphthalein monophosphate (PMP); tetramethyl benzidine (TMB); tetranitroblue tetrazolium (TNBT); X-Gal; X-Gluc; and X-Gluco side.

Other substrates can be used to produce products for local deposition that are luminescent. For example, in the presence of hydrogen peroxide (H₂O₂), horseradish peroxidase (HRP) can catalyze the oxidation of cyclic diacylhydrazides, such as luminol. Immediately following the oxidation, the luminol is in an excited state (intermediate reaction product), which decays to the ground state by emitting light. Strong enhancement of the light emission is produced by enhancers, such as phenolic compounds. Advantages include high sensitivity, high resolution, and rapid detection without radioactivity and requiring only small amounts of antibody. See, e.g., Thorpe et al., Methods Enzymol. 133: 331-53 (1986); Kricka et al., J. Immunoassay 17(1): 67-83 (1996); and Lundqvist et al., J. Biolumin. Chemilumin. 10(6): 353-9 (1995). Kits for such enhanced chemiluminescent detection (ECL) are available commercially. The antibodies can also be labeled using colloidal gold.

As another example, when the antibodies of the present invention are used, e.g., for flow cytometric detection, for scanning laser cytometric detection, or for fluorescent immunoassay, they can usefully be labeled with fluorophores. There are a wide variety of fluorophore labels that can usefully be attached to the antibodies of the present invention. For flow cytometric applications, both for extracellular detection and for intracellular detection, common useful fluorophores can be fluorescein isothiocyanate (FITC), allophycocyanin (APC), R-phycoerythrin (PE), peridinin chlorophyll protein (PerCP), Texas Red, Cy3, Cy5, fluorescence resonance energy tandem fluorophores such as PerCP-Cy5.5, PE-Cy5, PE-Cy5.5, PE-Cy7, PE-Texas Red, and APC-Cy7.

Other fluorophores include, inter alia, Alexa Fluor® 350, Alexa Fluor® 488, Alexa Fluor % 532, Alexa Fluor® 546, Alexa Fluor® 568, Alexa Fluor® 594, Alexa Fluor® 1647 (monoclonal antibody labeling kits available from Molecular Probes, Inc., Eugene, Oreg., USA), BODIPY dyes, such as BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, Texas Red (available from Molecular Probes, Inc., Eugene, Oreg., USA), and Cy2, Cy3, Cy3.5, Cy5, Cy5.5, and Cy7, all of which are also useful for fluorescently labeling the antibodies of the present invention. For secondary detection using labeled avidin, streptavidin, captavidin or neutravidin, the antibodies of the present invention can usefully be labeled with biotin.

When the antibodies of the present invention are used, e.g., for western blotting applications, they can usefully be labeled with radioisotopes, such as ³³P, ³²P, ³⁵S, ³H, and ¹²⁵I. As another example, when the antibodies of the present invention are used for radioimmunotherapy, the label can usefully be ²²⁸Th, ²²⁷Ac, ²²⁵Ac, ²²³Ra, ²¹³Bi, ²¹²Pb, ²¹² Bi, ²¹¹At, ²⁰³Pb, ¹⁹⁴OS, ¹⁸⁸Re, ¹⁸⁶Re, ¹⁵³Sm, ¹⁴⁹Tb, ¹³¹I, ¹²⁵I, ¹¹¹In, ¹⁰⁵Rh, ^{99m}Tc, ⁹⁷Ru, ⁹⁰Y, ⁹⁰Sr, ⁸⁸Y, ⁷²Se, ⁶⁷Cu, or ⁴⁷Sc.

As another example, when the antibodies of the present invention are to be used for in vivo diagnostic use, they can be rendered detectable by conjugation to MRI contrast agents, such as gadolinium diethylenetriaminepentaacetic acid (DTPA), Lauffer et al., Radiology 207(2): 529-38 (1998), or by radioisotopic labeling.

As would be understood, use of the labels described above is not restricted to the application as for which they were mentioned.

### Computer Readable Means

A further aspect of the invention is a computer readable means for storing the nucleic acid and amino acid sequences of the instant invention. In a preferred embodiment, the invention provides a computer readable means for storing the gene products of Table 2a and Table 2b and the gene products of Table 2a, Table 2b or Table 7 as described herein, as the complete set of sequences or in any combination. The records of the computer readable means can be accessed for reading and display and for interface with a computer system for the application of programs allowing for the location of data upon a query for data meeting certain criteria, the comparison of sequences, the alignment or ordering of sequences meeting a set of criteria, and the like.

### Diagnostic Methods for Colon Cancer

The present invention also relates to quantitative and qualitative diagnostic assays and methods for detecting, diagnosing, monitoring, staging and predicting colon cancer by comparing the expression of a CaSNA or a CaSP in a human patient that has or may have colon cancer, or who is at risk of developing colon cancer, with the expression of a CaSNA or a CaSP in a normal human control. For purposes of the present invention, “expression of a CaSNA” or “CaSNA expression” means the quantity of CaSNA mRNA that can be measured by any method known in the art or the level of transcription that can be measured by any method known in the art in a bodily fluid, cell, tissue, organ or whole patient. Similarly, the term “expression of a CaSP” or “CaSP expression” means the amount of CaSP that can be measured by any method known in the art or the level of translation of a CaSNA that can be measured by any method known in the art.

The present invention provides methods for diagnosing colon cancer in a patient, by analyzing for changes in levels of CaSNA or CaSP in cells, tissues, organs or bodily fluids compared with levels of CaSNA or CaSP in cells, tissues, organs or bodily fluids of preferably the same type from a normal human control, wherein an increase, or decrease in certain cases, in levels of a CaSNA or CaSP in the patient versus the normal human control is associated with the presence of colon cancer or with a predilection to the disease. In another preferred embodiment, the present invention provides methods for diagnosing colon cancer in a patient by analyzing changes in the structure of the mRNA of a CaSG compared to the mRNA from a normal control. These changes include, without limitation, aberrant splicing, alterations in polyadenylation and/or alterations in 5′ nucleotide capping. In yet another preferred embodiment, the present invention provides methods for diagnosing colon cancer in a patient by analyzing changes in a CaSP compared to a CaSP from a normal patient. These changes include, e.g., alterations, including post translational modifications such as glycosylation and/or phosphorylation of the CaSP or changes in the subcellular CaSP localization. These methods are particularly useful in diagnosing adenocarcinoma of the colon.

For purposes of the present invention, diagnosing means that CaSNA or CaSP levels are used to determine the presence or absence of disease in a patient. As will be understood by those of skill in the art, measurement of other diagnostic parameters may be required for definitive diagnosis or determination of the appropriate treatment for the disease. The determination may be made by a clinician, a doctor, a testing laboratory, or a patient using an over the counter test. The patient may have symptoms of disease or may be asymptomatic. In addition, the CaSNA or CaSP levels of the present invention may be used as screening marker to determine whether further tests or biopsies are warranted. In addition, the CaSNA or CaSP levels may be used to determine the vulnerability or susceptibility to disease.

In a preferred embodiment, the expression of a CaSNA is measured by determining the amount of a mRNA that encodes an amino acid sequence selected from the gene products of Table 2a and Table 2b, a homolog, an allelic variant, or a fragment thereof. In a more preferred embodiment, the CaSNA expression that is measured is the level of expression of a CaSNA mRNA selected from the gene products of Table 2a, Table 2b or Table 7, or a hybridizing nucleic acid, homologous nucleic acid or allelic variant thereof, or a part of any of these nucleic acid molecules. CaSNA expression may be measured by any method known in the art, such as those described supra, including measuring mRNA expression by Northern blot, quantitative or qualitative reverse transcriptase PCR (RT-PCR), microarray, dot or slot blots or in situ hybridization. See, e.g., Ausubel (1992), supra; Ausubel (1999), supra; Sambrook (1989), supra; and Sambrook (2001), supra. CaSNA transcription may be measured by any method known in the art including using a reporter gene hooked up to the promoter of a CaSG of interest or doing nuclear run-off assays. Alterations in mRNA structure, e.g., aberrant splicing variants, may be determined by any method known in the art, including, RT-PCR followed by sequencing or restriction analysis. As necessary, CaSNA expression may be compared to a known control, such as a normal colon nucleic acid, to detect a change in expression.

In another preferred embodiment, the expression of a CaSP is measured by determining the level of a CaSP having an amino acid sequence selected from the group consisting of the gene products of Table 2a and Table 2b, a homolog, an allelic variant, or a fragment thereof. Such levels are preferably determined in at least one of cells, tissues, organs and/or bodily fluids, including determination of normal and abnormal levels. Thus, for instance, a diagnostic assay in accordance with the invention for diagnosing over- or under-expression of a CaSNA or CaSP compared to normal control bodily fluids, cells, or tissue samples may be used to diagnose the presence of colon cancer. The expression level of a CaSP may be determined by any method known in the art, such as those described supra. In a preferred embodiment, the CaSP expression level may be determined by radioimmunoassays, competitive-binding assays, ELISA, Western blot, FACS, immunohistochemistry, immunoprecipitation, proteomic approaches: two-dimensional gel electrophoresis (2D electrophoresis) and non-gel-based approaches such as mass spectrometry or protein interaction profiling. See, e.g., Harlow (1999), supra; Ausubel (1992), supra; and Ausubel (1999), supra. Alterations in the CaSP structure may be determined by any method known in the art, including, e.g., using antibodies that specifically recognize phosphoserine, phosphothreonine or phosphotyrosine residues, two-dimensional polyacrylamide gel electrophoresis (2D PAGE) and/or chemical analysis of amino acid residues of the protein. Id.

In one embodiment, a radioimmunoassay (RIA) or an ELISA is used. An antibody specific to a CaSP is prepared if one is not already available. In a preferred embodiment, the antibody is a monoclonal antibody. The anti-CaSP antibody is bound to a solid support and any free protein binding sites on the solid support are blocked with a protein such as bovine serum albumin. A sample of interest is incubated with the antibody on the solid support under conditions in which the CaSP will bind to the anti-CaSP antibody. The sample is removed, the solid support is washed to remove unbound material, and an anti-CaSP antibody that is linked to a detectable reagent (a radioactive substance for RIA and an enzyme for ELISA) is added to the solid support and incubated under conditions in which binding of the CaSP to the labeled antibody will occur. After binding, the unbound labeled antibody is removed by washing. For an ELISA, one or more substrates are added to produce a colored reaction product that is based upon the amount of a CaSP in the sample. For an RIA, the solid support is counted for radioactive decay signals by any method known in the art. Quantitative results for both RIA and ELISA typically are obtained by reference to a standard curve.

Other methods to measure CaSP levels are known in the art. For instance, a competition assay may be employed wherein an anti-CaSP antibody is attached to a solid support and an allocated amount of a labeled CaSP and a sample of interest are incubated with the solid support. The amount of labeled CaSP attached to the solid support can be correlated to the quantity of a CaSP in the sample.

Expression levels of a CaSNA can be determined by any method known in the art, including PCR and other nucleic acid methods, such as ligase chain reaction (LCR) and nucleic acid sequence based amplification (NASBA). Reverse-transcriptase PCR (RT-PCR) is a powerful technique which can be used to detect the presence of a specific mRNA population in a complex mixture of thousands of other mRNA species. In RT-PCR, an mRNA species is first reverse transcribed to complementary DNA (cDNA) with use of the enzyme reverse transcriptase; the cDNA is then amplified as in a standard PCR reaction.

Hybridization to specific DNA molecules (e.g., oligonucleotides) arrayed on a solid support can be used to both detect the expression of and quantitate the level of expression of one or more CaSNAs of interest. In this approach, all or a portion of one or more CaSNAs is fixed to a substrate. A sample of interest, which may comprise RNA, e.g., total RNA or polyA-selected mRNA, or a complementary DNA (cDNA) copy of the RNA is incubated with the solid support under conditions in which hybridization will occur between the DNA on the solid support and the nucleic acid molecules in the sample of interest. Hybridization between the substrate-bound DNA and the nucleic acid molecules in the sample can be detected and quantitated by several means, including, without limitation, radioactive labeling or fluorescent labeling of the nucleic acid molecule or a secondary molecule designed to detect the hybrid.

The above tests can be carried out on samples derived from a variety of cells, bodily fluids and/or tissue extracts such as homogenates or solubilized tissue obtained from a patient. Tissue extracts are obtained routinely from tissue biopsy and autopsy material. Bodily fluids useful in the present invention include blood, urine, saliva, feces or any other bodily secretion or derivative thereof. As used herein “blood” includes whole blood, plasma, serum, circulating epithelial cells, constituents, or any derivative of blood.

In addition to detection in bodily fluids, the proteins and nucleic acids of the invention are suitable to detection by cell capture technology. Whole cells may be captured by a variety methods. For example, magnetic separation as described in U.S. Pat. Nos. 5,200,084; 5,186,827; 5,108,933; 4,925,788, the disclosures of which are incorporated herein by reference in their entireties can be used to capture whole cells. Epithelial cells may be captured using such products as Dynabeads® or CELLection™ (Dynal Biotech, Oslo, Norway). Alternatively, fractions of blood may be captured, e.g., the buffy coat fraction (50 mm cells isolated from 5 ml of blood) containing epithelial cells. In addition, cancer cells may be captured using the techniques described in WO 00/47998, the disclosure of which is incorporated herein by reference in its entirety. Once the cells are captured or concentrated, the proteins or nucleic acids are detected by means described herein. Alternatively, nucleic acids may be captured directly from blood samples, see U.S. Pat. Nos. 6,156,504, 5,501,963; or WO 01/42504, the disclosures of which are incorporated herein by reference in their entireties.

In a preferred embodiment, the specimen tested for expression of CaSNA or CaSP comprises normal or cancerous colon tissue, normal or cancerous colon cells grown in cell culture, blood, serum, lymph node tissue, or lymphatic fluid. Fecal specimens can also be tested for the present of a CaSNA or CaSP of the present invention. In another preferred embodiment, especially when metastasis of primary colon cancer is known or suspected, specimens include, without limitation, tissues from brain, bone, bone marrow, liver, lungs, breast, and adrenal glands. In general, the tissues may be sampled by biopsy, including, without limitation, needle biopsy, e.g., transthoracic needle aspiration, cervical mediatinoscopy, endoscopic lymph node biopsy, video-assisted thoracoscopy, exploratory thoracotomy, bone marrow biopsy and bone marrow aspiration.

All the methods of the present invention may optionally include determining the expression levels of one or more other cancer markers in addition to determining the expression level of a CaSNA or CaSP. In many cases, the use of another cancer marker will decrease the likelihood of false positives or false negatives. In one embodiment, the one or more other cancer markers include other CaSNA or CaSPs as disclosed herein. In a preferred embodiment, at least one other cancer marker in addition to a particular CaSNA or CaSP is measured. In a more preferred embodiment, at least two other additional cancer markers are used. In an even more preferred embodiment, at least three, more preferably at least five, even more preferably at least ten additional cancer markers are used.

In a preferred embodiment, the specimen tested for expression of CaSNA or CaSP includes without limitation colon tissue, fecal samples, colonocytes, colon cells grown in cell culture, blood, serum, lymph node tissue, and lymphatic fluid.

Colonocytes represent an important source of the CaSP or CaSNAs because they provide a picture of the immediate past metabolic history of the GI tract of a subject. In addition, such cells are representative of the cell population from a statistically large sampling frame reflecting the state of the colonic mucosa along the entire length of the colon in a non-invasive manner, in contrast to a limited sampling by colonic biopsy using an invasive procedure involving endoscopy. Specific examples of patents describing the isolation of colonocytes include U.S. Pat. Nos. 6,335,193; 6,020,137 5,741,650; 6,258,541; US 2001 0026925 A1; WO 00/63358 A1, the disclosures of which are incorporated herein by reference in their entireties.

Diagnosing

In one aspect, the invention provides a method for determining the expression levels and/or structural alterations of one or more CaSNA and/or CaSP in a sample from a patient suspected of having colon cancer. In general, the method comprises the steps of obtaining the sample from the patient, determining the expression level or structural alterations of a CaSNA and/or CaSP and then ascertaining whether the patient has colon cancer from the expression level of the CaSNA or CaSP. In general, if high expression relative to a control of a CaSNA or CaSP is indicative of colon cancer, a diagnostic assay is considered positive if the level of expression of the CaSNA or CaSP is at least one and a half times higher, and more preferably are at least two times higher, still more preferably five times higher, even more preferably at least ten times higher, than in preferably the same cells, tissues or bodily fluid of a normal human control. In contrast, if low expression relative to a control of a CaSNA or CaSP is indicative of colon cancer, a diagnostic assay is considered positive if the level of expression of the CaSNA or CaSP is at least one and a half times lower, and more preferably are at least two times lower, still more preferably five times lower, even more preferably at least ten times lower than in preferably the same cells, tissues or bodily fluid of a normal human control. The normal human control may be from a different patient or from uninvolved tissue of the same patient.

The present invention also provides a method of determining whether colon cancer has metastasized in a patient. One may identify whether the colon cancer has metastasized by measuring the expression levels and/or structural alterations of one or more CaSNAs and/or CaSPs in a variety of tissues. The presence of a CaSNA or CaSP in a certain tissue at levels higher than that of corresponding noncancerous tissue (e.g., the same tissue from another individual) is indicative of metastasis if high level expression of a CaSNA or CaSP is associated with colon cancer. Similarly, the presence of a CaSNA or CaSP in a tissue at levels lower than that of corresponding noncancerous tissue is indicative of metastasis if low level expression of a CaSNA or CaSP is associated with colon cancer. Further, the presence of a structurally altered CaSNA or CaSP that is associated with colon cancer is also indicative of metastasis.

In general, if high expression relative to a control of a CaSNA or CaSP is indicative of metastasis, an assay for metastasis is considered positive if the level of expression of the CaSNA or CaSP is at least one and a half times higher, and more preferably are at least two times higher, still more preferably five times higher, even more preferably at least ten times higher, than in preferably the same cells, tissues or bodily fluid of a normal human control. In contrast, if low expression relative to a control of a CaSNA or CaSP is indicative of metastasis, an assay for metastasis is considered positive if the level of expression of the CaSNA or CaSP is at least one and a half times lower, and more preferably are at least two times lower, still more preferably five times lower, even more preferably at least ten times lower than in preferably the same cells, tissues or bodily fluid of a normal human control.

Staging

The invention also provides a method of staging colon cancer in a human patient. The method comprises identifying a human patient having colon cancer and analyzing cells, tissues or bodily fluids from such human patient for expression levels and/or structural alterations of one or more CaSNAs or CaSPs. First, one or more tumors from a variety of patients are staged according to procedures well known in the art, and the expression levels of one or more CaSNAs or CaSPs is determined for each stage to obtain a standard expression level for each CaSNA and CaSP. Then, the CaSNA or CaSP expression levels of the CaSNA or CaSP are determined in a biological sample from a patient whose stage of cancer is not known. The CaSNA or CaSP expression levels from the patient are then compared to the standard expression level. By comparing the expression level of the CaSNAs and CaSPs from the patient to the standard expression levels, one may determine the stage of the tumor. The same procedure may be followed using structural alterations of a CaSNA or CaSP to determine the stage of a colon cancer.

Monitoring

Further provided is a method of monitoring colon cancer in a human patient. One may monitor a human patient to determine whether there has been metastasis and, if there has been, when metastasis began to occur. One may also monitor a human patient to determine whether a preneoplastic lesion has become cancerous. One may also monitor a human patient to determine whether a therapy, e.g., chemotherapy, radiotherapy or surgery, has decreased or eliminated the colon cancer. The monitoring may determine if there has been a reoccurrence and, if so, determine its nature. The method comprises identifying a human patient that one wants to monitor for colon cancer, periodically analyzing cells, tissues or bodily fluids from such human patient for expression levels of one or more CaSNAs or CaSPs, and comparing the CaSNA or CaSP levels over time to those CaSNA or CaSP expression levels obtained previously. Patients may also be monitored by measuring one or more structural alterations in a CaSNA or CaSP that are associated with colon cancer.

If increased expression of a CaSNA or CaSP is associated with metastasis, treatment failure, or conversion of a preneoplastic lesion to a cancerous lesion, then detecting an increase in the expression level of a CaSNA or CaSP indicates that the tumor is metastasizing, that treatment has failed or that the lesion is cancerous, respectively. One having ordinary skill in the art would recognize that if this were the case, then a decreased expression level would be indicative of no metastasis, effective therapy or failure to progress to a neoplastic lesion. If decreased expression of a CaSNA or CaSP is associated with metastasis, treatment failure, or conversion of a preneoplastic lesion to a cancerous lesion, then detecting a decrease in the expression level of a CaSNA or CaSP indicates that the tumor is metastasizing, that treatment has failed or that the lesion is cancerous, respectively. In a preferred embodiment, the levels of CaSNAs or CaSPs are determined from the same cell type, tissue or bodily fluid as prior patient samples. Monitoring a patient for onset of colon cancer metastasis is periodic and preferably is done on a quarterly basis, but may be done more or less frequently.

The methods described herein can further be utilized as prognostic assays to identify subjects having or at risk of developing a disease or disorder associated with increased or decreased expression levels of a CaSNA and/or CaSP. The present invention provides a method in which a test sample is obtained from a human patient and one or more CaSNAs and/or CaSPs are detected. The presence of higher (or lower) CaSNA or CaSP levels as compared to normal human controls is diagnostic for the human patient being at risk for developing cancer, particularly colon cancer. The effectiveness of therapeutic agents to decrease (or increase) expression or activity of one or more CaSNAs and/or CaSPs of the invention can also be monitored by analyzing levels of expression of the CaSNAs and/or CaSPs in a human patient in clinical trials or in in vitro screening assays such as in human cells. In one example, the over-expression of gene products selected from the group comprising CYR61 (Table 2a) and TYMS, TK1, and DTYMK (Table 2b) are indicative of a cancer phenotype resistant to fluorouracil. In this way, the gene expression pattern can serve as a marker, indicative of the physiological response of the human patient or cells, as the case may be, to the agent being tested.

### Methods of Detecting Noncancerous Diseases of the Colon

The present invention also provides methods for determining the expression levels and/or structural alterations of one or more CaSNAs and/or CaSPs in a sample from a patient suspected of having or known to have a noncancerous disease of the colon. In general, the method comprises the steps of obtaining a sample from the patient, determining the expression level or structural alterations of a CaSNA and/or CaSP, comparing the expression level or structural alteration of the CaSNA or CaSP to a normal colon control, and then ascertaining whether the patient has a noncancerous colon disease. In general, if high expression relative to a control of a CaSNA or CaSP is indicative of a particular noncancerous colon disease, a diagnostic assay is considered positive if the level of expression of the CaSNA or CaSP is at least two times higher, more preferably at least five times higher, and even more preferably at least ten times higher, than in preferably the same cells, tissues or bodily fluid of a normal human control. In contrast, if low expression relative to a control of a CaSNA or CaSP is indicative of a noncancerous colon disease, a diagnostic assay is considered positive if the level of expression of the CaSNA or CaSP is at least two times lower, more preferably at least five times lower, and even more preferably at least ten times lower than in preferably the same cells, tissues or bodily fluid of a normal human control. The normal human control may be from a different patient or from uninvolved tissue of the same patient.

One having ordinary skill in the art may determine whether a CaSNA and/or CaSP is associated with a particular noncancerous colon disease by obtaining colon tissue from a patient having a noncancerous colon disease of interest and determining which CaSNAs and/or CaSPs are expressed in the tissue at either a higher or a lower level than in normal colon tissue. In another embodiment, one may determine whether a CaSNA or CaSP exhibits structural alterations in a particular noncancerous colon disease by obtaining colon tissue from a patient having a noncancerous colon disease of interest and determining the structural alterations in one or more CaSNAs and/or CaSPs relative to normal colon tissue.

### Methods for Identifying Colon Tissue

In another aspect, the invention provides methods for identifying colon tissue. These methods are particularly useful in, e.g., forensic science, colon cell differentiation and development, and in tissue engineering.

In one embodiment, the invention provides a method for determining whether a sample is colon tissue or has colon tissue-like characteristics. The method comprises the steps of providing a sample suspected of comprising colon tissue or having colon tissue-like characteristics, determining whether the sample expresses one or more CaSNAs and/or CaSPs, and, if the sample expresses one or more CaSNAs and/or CaSPs, concluding that the sample comprises colon tissue. In a preferred embodiment, the CaSNA encodes a polypeptide having an amino acid sequence selected from the gene products of Table 2a and Table 2b, or a homolog, allelic variant or fragment thereof. In a more preferred embodiment, the CaSNA has a nucleotide sequence selected from the gene products of Table 2a, Table 2b or Table 7, or a hybridizing nucleic acid, an allelic variant or a part thereof. Determining whether a sample expresses a CaSNA can be accomplished by any method known in the art. Preferred methods include hybridization to microarrays, Northern blot hybridization, and quantitative or qualitative RT-PCR. In another preferred embodiment, the method can be practiced by determining whether a CaSP is expressed. Determining whether a sample expresses a CaSP can be accomplished by any method known in the art. Preferred methods include Western blot, ELISA, RIA and 2D PAGE. In one embodiment, the CaSP has an amino acid sequence selected from the gene products of Table 2a and Table 2b, or a homolog, allelic variant or fragment thereof. In another preferred embodiment, the expression of at least two CaSNAs and/or CaSPs is determined. In a more preferred embodiment, the expression of at least three, more preferably four and even more preferably five CaSNAs and/or CaSPs are determined.

In another embodiment, an anti-CaSP antibody may be linked to an imaging agent that can be detected using, e.g., magnetic resonance imaging, CT or PET. This would be useful for determining and monitoring colon function, identifying colon cancer tumors, and identifying noncancerous colon diseases.

### Articles of Manufacture and Kits

The invention also relates to an article of manufacture containing materials useful for the detection gene products of Table 2a and Table 2b. Such material may detect nucleic acids such as DNA and RNA or amino acids such as proteins or peptides. The article of manufacture comprises a container and a composition contained therein comprising nucleic acid primers and probes specific for the gene products of this invention. Alternatively, the article of manufacture comprises a container and a composition contained therein comprising an antibody specific for the gene products of this invention. The article of manufacture may also comprise a label or package insert on or associated with the container. Suitable containers include, for example, bottles, vials, syringes, etc. The containers may be formed from a variety of materials such as glass or plastic. The container holds a composition which is effective for detecting The label or package insert indicates that the composition is used for prognosing, detecting or staging colon cancer, in an individual in need thereof. The label or package insert may further comprise instructions for detecting a gene product in a sample from an individual. The label or package insert may provide a description of the composition as well as instructions for the intended in vitro or diagnostic use. Additionally, the article of manufacture may further comprise a second container comprising a substance which detects the antibody of this invention, e.g., a second antibody which binds to the antibodies of this invention. The substance may be labeled with a detectable label such as those disclosed herein. The article of manufacture may further include other materials desirable from a commercial and user standpoint, including other buffers, diluents, filters, needles, and syringes.

### EXAMPLES

### Example 1a

### Differentially Expressed Gene Products in Colon Cancer

For the detection of cancer or stratification of individuals into groups predicted to have different disease outcomes, the expression levels of gene products were determined. Genes were selected based on individual expression profiles and functional relevance of the encoded protein as described by gene ontology and the literature. Genes within the functionally relevant groups below are likely to be useful for (1) detection of cancer, (2) stratification of individuals into groups predicted to have different disease outcomes; (3) selection of individuals for a particular therapeutic intervention; or identification of individuals responding to a therapeutic regimen.

TABLE 1

Extracellular matrix
Cell adhesion
Regulation of transcription
Ubiquitination
Lipid metabolism
Signal transduction
DNA repair
Immune response
Transport
Chemotaxis
G-protein couple receptor
Apoptosis
Cell recognition
Anti-apoptosis
beta catenin

A gene product associated with one or more of the functional categories above will be particularly useful if it has one or more of the following properties: structural and/or physical, chemical or enzymatic, regulatory, signal transduction, or ligand, receptor or substrate binding. In addition, genes or gene products directly involved in the sequential and organ specific development of cancer are of interest.

Based on the criteria above, we identified a set of genes and associated gene products. Table 2a and Table 2b below provide a summary of these genes including: the Genebank Accessions (ncbi with the extension .nlm.nih.gov of the world wide web), the abbreviated common name for the genes, internal identifiers, functional association(s) for the gene product and annotation of the gene from public databases (e.g. GeneBank).

In addition, Table 3 below contains the Genebank Accession, the chromosomal location of the gene (with amplification or loss of homology annotation), Gene Ontology (GO) ID/classifications including: Cellular Component Ontology, Molecular Function Ontology and Biological Process Ontology. Also included is a description of gene product function derived from the literature. References supporting GO and functional annotations of the Genbank Accession in Table 3 are available in public databases such as Genebank and Swissprot (expasy with the extension .org of the world wide web).

TABLE 2a

| Genebank | Abbreviated | DDXS amplicon | |
|---|---|---|---|
| Accession | Name | name | Annotation |
| NM_032044.2 | REGIV | Cln101 | Homo sapiens regenerating islet-derived family, member 4 (REG4), mRNA. |
| NM_007052.3 | NOX1 | Cln106 | Homo sapiens NADPH oxidase 1 (NOX1), transcript variant NOH-1L, mRNA. |
| NM_004363.1 | CEACAM5 | Cln224v1 | Homo sapiens carcinoembryonic antigen-related cell adhesion molecule 5 |
| | | | (CEACAM5), mRNA |
| NM_033229.1 | TRIM15 | Cln129 | Homo sapiens tripartite motif-containing 15 (TRIM15), transcript variant 1, mRNA |
| AC023992.8 | RNF43 | Cln242v1 | Homo sapiens chromosome 17, clone RP11-247I5, complete sequence. |
| AL359752.11 | REGIV-like | Cln101V1 | Human DNA sequence from clone RP5-1042I8 on chromosome 1p11-13.2 Contains |
| | protein | | the REG4 gene for regenerating islet-derived family member 4, a novel pseudogene, |
| | | | a profilin 1 (PFN1) pseudogene, the ADAM30 gene for a disintegrin and |
| | | | metalloproteinase domain 30 and the 3′ end of the NOTCH2 gene for Notch |
| | | | homolog 2 (Drosophila), complete sequence. |
| NM_080748.1 | C20orf52 | Cln254 | Homo sapiens chromosome 20 open reading frame 52 (C20orf52), mRNA |
| NM_080748.1 | C20orf52 | Cln254a | Homo sapiens chromosome 20 open reading frame 52 (C20orf52), mRNA |
| NM_138805.2 | FAM3D | Cln108 | Homo sapiens family with sequence similarity 3, member D (FAM3D), mRNA |
| NM_138805.2 | FAM3D | Cln108b | Homo sapiens family with sequence similarity 3, member D (FAM3D), mRNA |
| NM_138805.2 | FAM3D | Cln108c | Homo sapiens family with sequence similarity 3, member D (FAM3D), mRNA |
| NM_006418.3 | OLFM4 | Cln109c | Homo sapiens olfactomedin 4 (OLFM4), mRNA |
| NM_006418.3 | OLFM4 | Cln109 | Homo sapiens olfactomedin 4 (OLFM4), mRNA |
| NM_006418.3 | OLFM4 | Cln109B | Homo sapiens olfactomedin 4 (OLFM4), mRNA |
| NM_024017.3 | HOXB9 | Cln130 | Homo sapiens homeo box B9 (HOXB9), mRNA |
| NM_024017.3 | HOXB9 | Cln130a | Homo sapiens homeo box B9 (HOXB9), mRNA |
| NM_006149.2 | GAL4 | Cln114 | Homo sapiens lectin, galactoside-binding, soluble, 4 (galectin 4) (LGALS4), mRNA |
| NM_001738.1; | CA1 | Cln115 | Homo sapiens carbonic anhydrase I (CA1), mRNA |
| M33987.1 | | | |
| AY358469.1 | UNQ511 | Cln124 | Homo sapiens clone DNA59613 phospholipase inhibitor (UNQ511) mRNA |
| NM_017716.1 | MS4A12 | Cln125 | Homo sapiens membrane-spanning 4-domains, subfamily A, member 12 (MS4A12), |
| | | | mRNA |
| NM_002644.2 | PIGR | Cln113 | Homo sapiens polymeric immunoglobulin receptor (PIGR), mRNA |
| NM_017625.2 | ITLN1 | DSH505 | Homo sapiens intelectin 1 (galactofuranose binding) (ITLN1), mRNA. |
| NM_031457.1 | MS4A8B | DSH510 | Homo sapiens membrane-spanning 4-domains, subfamily A, member 8B (MS4A8B), |
| | | | mRNA. |
| NM_005727.2 | TSPAN1 | DSH522 | Homo sapiens tetraspanin 1 (TSPAN1), mRNA |
| NM_003823.2 | TNFRSF6B, | Cln248 | Homo sapiens tumor necrosis factor receptor superfamily, member 6b, decoy |
| | DCR3 | | (TNFRSF6B), transcript variant M68E, mRNA |
| NM_001415.2 | EIF2S3 | Cln243 | Homo sapiens eukaryotic translation initiation factor 2, subunit 3 gamma, 52 kDa |
| | | | (EIF2S3), mRNA. |
| NM_012155.1 | EML2 | Cln264 | Homo sapiens echinoderm microtubule associated protein like 2 (EML2), mRNA |
| NM_000582.2 | SPP1 | Cln245 | Homo sapiens secreted phosphoprotein 1 (osteopontin, bone sialoprotein I, early |
| | | | T-lymphocyte activation 1) (SPP1), mRNA |
| NM_032023.3 | RASSF4 | Ovr216 | Homo sapiens Ras association (RaIGDS/AF-6) domain family 4 (RASSF4), transcript |
| | | | variant 1, mRNA |
| NM_144947.1 | KLK11 | DSH38 | Homo sapiens kallikrein 11 (KLK11), transcript variant 2, mRNA |
| AC084847.5 | NA | Cln237v1 | Homo sapiens chromosome 8, clone CTD-2343B20, complete sequence. |
| NM_017763.3; | RNF43; URCC | Cln242 | Homo sapiens ring finger protein 43 (RNF43), mRNA.; Homo sapiens hypothetical |
| AB081837.1 | | | protein FLJ20315 (FLJ20315), mRNA |
| AJ236922.1 | mGluR8c | Cln260 | Homo sapiens mRNA for metabotropic glutamate receptor 8c. |
| NM_002483.3 | CEACAM6 | Cln263 | Homo sapiens carcinoembryonic antigen-related cell adhesion molecule 6 (non-specific |
| | | | cross reacting antigen) (CEACAM6), mRNA |
| NM_006408.2 | AGR2 | Mam111 | Homo sapiens anterior gradient 2 homolog (Xenopus laevis) (AGR2), mRNA |
| NM_004864.1 | GDF15 | Pcan065 | Homo sapiens growth differentiation factor 15 (GDF15), mRNA. |
| NM_012445.1 | SPON2 | Pro108a | Homo sapiens spondin 2, extracellular matrix protein (SPON2), mRNA. |
| NM_138938.1 | REG3A | Pcan041 | Homo sapiens regenerating islet-derived 3 alpha (REG3A), transcript variant 2, mRNA |
| BC070213.1 | SLAMF9 | Pcan047b | Homo sapiens SLAM family member 9, mRNA (cDNA clone IMAGE: 30416664), |
| | | | complete cds. |
| NM_006475.1 | POSTN | Cln252 | Homo sapiens periostin, osteoblast specific factor (POSTN), mRNA. |
| NM_004385.2 | CSPG2 | Pcan045 | Homo sapiens chondroitin sulfate proteoglycan 2 (versican) (CSPG2), mRNA. |
| NM_004385.2 | CSPG2 | Pcan045b | Homo sapiens chondroitin sulfate proteoglycan 2 (versican) (CSPG2), mRNA. |
| BC021275.2 | PACAP | Pcan039b | Homo sapiens proapoptotic caspase adaptor protein, mRNA (cDNA clone MGC: 29506 |
| | | | IMAGE: 4853250), complete cds. |
| NM_005408.2 | CCL13 | DSH82/83 | Homo sapiens chemokine (C-C motif) ligand 13 (CCL13), mRNA |
| NM_018098.4 | ECT2 | Cln176b | Homo sapiens epithelial cell transforming sequence 2 oncogene (ECT2), mRNA. |
| NM_006645.1 | STARD10 | DEX0451_037.nt.3 | Homo sapiens START domain containing 10 (STARD10), Mrna |
| NM_004625.3 | WNT7A | Ovr212a | Homo sapiens wingless-type MMTV integration site family, member 7A (WNT7A), |
| | | | mRNA |
| NM_001008540.1 | CXCR4 | DSH862 | Homo sapiens chemokine (C—X—C motif) receptor 4 (CXCR4), transcript variant 1, |
| | | | mRNA. |
| NM_000579.1 | CCR5 | DSH51 | Homo sapiens chemokine (C-C motif) receptors (CCR5), mRNA. |
| NM_004367.3 | CCR6 | DSH106 | Homo sapiens chemokine (C-C motif) receptor 6 (CCR6), transcript variant 1, mRNA. |
| NM_004591.1 | CCL20 | DSH73 | Homo sapiens chemokine (C-C motif) ligand 20 (CCL20), mRNA. |
| NM_006564.1 | CXCR6 | DSH105 | Homo sapiens chemokine (C—X—C motif) receptor 6 (CXCR6), mRNA. |
| NM_178445.1 | CCRL1 | DSH97 | Homo sapiens chemokine (C-C motif) receptor-like 1 (CCRL1), transcript variant 1, |
| | | | mRNA. |
| NM_003965.3 | CCRL2 | DSH209 | Homo sapiens chemokine (C-C motif) receptor-like 2 (CCRL2), mRNA. |
| NM_001838.2 | CCR7 | DSH859 | Homo sapiens chemokine (C-C motif) receptor 7 (CCR7), mRNA. |
| NM_002989.2 | CCL21 | DSH89 | Homo sapiens chemokine (C-C motif) ligand 21 (CCL21), mRNA. |
| NM_001554.3 | CYR61 | Ovr235c | Homo sapiens cysteine-rich, angiogenic inducer, 61 (CYR61), mRNA |
| AY327584.1 | MUC1/S2 | Mam096 | Homo sapiens mucin short variant S2 (MUC1) mRNA, complete cds. |
| NM_006988.3 | ADAMTS1 | DSH607 | Homo sapiens a disintegrin-like and metalloprotease (reprolysin type) with |
| | | | thrombospondin type 1 motif, 1 (ADAMTS1), mRNA. |
| NM_001571.2 | IRF3 | DSH371 | Homo sapiens interferon regulatory factor 3 (IRF3), mRNA. |
| NM_145306.1 | C10orf35 | Pcan035 | Homo sapiens chromosome 10 open reading frame 35 (C10orf35), mRNA. |
| BC042754.1 | LOC143458 | DSH196 | Homo sapiens hypothetical protein LOC143458, mRNA (cDNA clone IMAGE: |
| | | | 4828259), partial cds. |
| NM_001908.3 | CTSB | DSH223/CTSB | Homo sapiens cathepsin B (CTSB), transcript variant 1, mRNA |
| NM_031419.2 | NFKBIZ | DSH198 | Homo sapiens nuclear factor of kappa light polypeptide gene enhancer in B-cells |
| | | | inhibitor, zeta (NFKBIZ), transcript variant 1, mRNA. |
| NM_006096.2 | NDRG1 | DSH207 | Homo sapiens N-myc downstream regulated gene 1 (NDRG1), mRNA |
| NM_006096.2 | NDRG1 | DSH207a | Homo sapiens N-myc downstream regulated gene 1 (NDRG1), mRNA |
| NM_207520.1 | RTN4 | DSH211 | Homo sapiens reticulon 4 (RTN4), transcript variant 4, mRNA |
| NM_005063.4 | SCD | DSH226 | Homo sapiens stearoyl-CoA desaturase (delta-9-desaturase) (SCD), mRNA |
| NM_198976.1 | TH1L | DSH248 | Homo sapiens TH1-like (Drosophila) (TH1L), transcript variant 1, mRNA |
| CR749471.1 | DKFZp781I1117 | DSH250 | Homo sapiens mRNA; cDNA DKFZp781I1117 (from clone DKFZp781I1117). |
| CR749471.1 | DKFZp781I1117 | DSH250a | Homo sapiens mRNA; cDNA DKFZp781I1117 (from clone DKFZp781I1117). |
| AC021236.10 | Clone: RP11- | DSH260 | Homo sapiens chromosome 8, clone RP11-113H14, complete sequence |
| | 113H14 | | |
| NM_024918.2 | C20orf172 | DSH279 | Homo sapiens chromosome 20 open reading frame 172 (C20orf172), mRNA |
| AC093619.5 | RP13-741A20 | DSH282 | Homo sapiens BAC clone RP13-741A20 from 7, complete sequence |
| NM_005564.2 | LCN2 | DSH330 | Homo sapiens lipocalin 2 (oncogene 24p3) (LCN2), mRNA. |
| AY623117.1 | RAD54-like | DSH811a | Homo sapiens RAD54-like (S. cerevisiae) (RAD54L) gene, complete cds. |
| NM_005201.2 | CCR8 | DSH375 | Homo sapiens chemokine (C-C motif) receptor 8 (CCR8), mRNA. |
| NM_139276.2 | STAT3 | DSH265 | Homo sapiens signal transducer and activator of transcription 3 (acute-phase response |
| | | | factor) (STAT3), transcript variant 1, mRNA. |

TABLE 2b

| | | DDXS | |
|---|---|---|---|
| Genebank | Abbreviated | amplicon | |
| Accession | Name | name | Annotation |
| NM_004994.1 | MMP9 | MMP9 | Homo sapiens matrix metalloproteinase 9 (gelatinase B, 92 kDa gelatinase, 92 kDa type IV collagenase) |
| | | | (MMP9), mRNA. |
| NM_003219.1 | TERT | TERT | Homo sapiens telomerase reverse transcriptase (TERT), transcript variant 1, mRNA. |
| NM_001071.1 | TYMS | TS | Homo sapiens thymidylate synthetase (TYMS), mRNA. |
| NM_198496.1 | AMACO | AMACO | Homo sapiens A-domain containing protein similar to matrilin and collagen (AMACO), mRNA. |
| NM_199168.1 | CXCL12 | CXCL12 | Homo sapiens chemokine (C—X—C motif) ligand 12 (stromal cell-derived factor 1) (CXCL12), mRNA. |
| NM_022059.1 | CXCL16 | CXCL16 | Homo sapiens chemokine (C—X—C motif) ligand 16 (CXCL16), mRNA. |
| NM_003376.3 | VEGF | VEGF | Homo sapiens vascular endothelial growth factor (VEGF), mRNA. |
| NM_004363.1 | CEACAM5 | CEACAM5 | Homo sapiens carcinoembryonic antigen-related cell adhesion molecule 5 (CEACAM5), mRNA |
| NM_019010.1 | KRT20 | KRT20 | Homo sapiens keratin 20 (KRT20), mRNA. |
| NM_006636.2 | MTHFD2 | MTHFD2 | Homo sapiens methylenetetrahydrofolate dehydrogenase (NADP+ dependent) 2, methenyltetrahydrofolate |
| | | | cyclohydrolase (MTHFD2), nuclear gene encoding mitochondrial protein, mRNA. |
| NM_003258.1 | TK1 | TK1 | Homo sapiens thymidine kinase 1, soluble (TK1), mRNA |
| NM_012145.2 | DTYMK | DTYMK | Homo sapiens deoxythymidylate kinase (thymidylate kinase) (DTYMK), mRNA |
| NM_000610.3 | CD44 | CD44 | Homo sapiens CD44 antigen (homing function and Indian blood group system) (CD44), transcript |
| | | | variant 1, mRNA. |
| NM_198175.1 | NME1 | NME1 | Homo sapiens non-metastatic cells 1, protein (NM23A) expressed in (NME1), transcript variant 1, mRNA. |
| NM_002466.2 | MYBL2 | MYBL2 | Homo sapiens v-myb myeloblastosis viral oncogene homolog (avian)-like 2 MYBL2, mRNA. |
| NM_001255.1 | CDC20 | CDC20 | Homo sapiens CDC20 cell division cycle 20 homolog (S. cerevisiae) (CDC20), mRNA. |
| NM_004413.1 | DPEP1 | DPEP1 | Homo sapiens dipeptidase 1 (renal) (DPEP1), mRNA. |
| NM_003270.2 | TSPN6 | TSPAN6 | Homo sapiens tetraspanin 6 (TSPAN6), mRNA. |
| NM_080820.3 | HARS2 | HARS2 | Homo sapiens histidyl-tRNA synthetase 2 (HARS2), mRNA. |
| NM_006649.2 | UTP14A | UTP14A | Homo sapiens UTP14, U3 small nucleolar ribonucleoprotein, homolog A (yeast) (UTP14A), mRNA. |
| NM_005804.2 | DDX39 | DDX39 | Homo sapiens DEAD (Asp-Glu-Ala-Asp) box polypetide 39 (DDX39), transcript variant 1, mRNA. |
| NM_003153.3 | STAT6 | STAT6 | Homo sapiens signal transducer and activator of transcription 6, interleukin-4 induced (STAT6), mRNA. |

TABLE 3

| Genebank Accession | Chr Loc | Cellular Component Ontology | Molecular Function Ontology | Biological Process Ontology | Literature Function |
|---|---|---|---|---|---|
| NM_032044.2 | 1p13.1-p12 | NA | sugar binding [goid 0005529] [evidence | | Results suggest that RELP might |
| | | | IEA] | | be involved in inflammatory and |
| | | | | | metaplastic responses of the |
| | | | | | gastrointestinal epithelium. |
| NM_007052.3 | Xq22 | go_component: membrane | go_function: oxidoreductase activity [goid | go_process: ion transport [goid | Nuclear factor (NF)-kappaB was |
| | | [goid 0016020] [evidence | 0016491] [evidence IEA]; go_function: | 0006811] [evidence IEA]; | predominantly activated in |
| | | IEA]; go_component: | voltage-gated proton channel activity | go_process: NADP metabolism [goid | adenoma and adenocarcinoma |
| | | integral to membrane [goid | [goid 0030171] [evidence TAS] [pmid | 0006739] [evidence NAS]; | cells expressing abundant Nox1, |
| | | 0016021] [evidence NAS] | 10615049]; go_function: superoxide- | go_process: FADH2 metabolism | suggesting that Nox1 may |
| | | | generating NADPH oxidase activity [goid | [goid 0006746] [evidence NAS]; | stimulate NF-kappaB-dependent |
| | | | 0016175] [evidence TAS] [pmid | go_process: electron transport [goid | antiapoptotic pathways in colon tumors. |
| | | | 10485709] | 0006118] [evidence NAS]; | |
| | | | | go_process: proton transport [goid | |
| | | | | 0015992] [evidence TAS] [pmid 10615049]” | |
| NM_004363.1 | 19q13.1-q13.2 | membrane [goid 0016020] | Interacting selectively with any | NA | NA |
| | | [evidence IEA]; integral to | glycosylphosphatidylinositol anchor. GPI | | |
| | | plasma membrane [goid | anchors serve to attach membrane | | |
| | | 0005887] [evidence TAS] | proteins to the lipid bilayer of cell | | |
| | | [pmid 3814146] | membranes [goid 0048503] | | |
| NM_033229.1 | 6p21.3 | ubiquitin ligase complex | transcription factor activity [goid 0003700] | protein ubiquitination [goid 0016567] | NA |
| | | [goid 0000151] [evidence | [evidence NR]; ubiquitin-protein ligase | [evidence IEA]; mesodermal cell fate | |
| | | IEA] | activity [goid 0004842] [evidence IEA] | determination [goid 0007500] | |
| | | | | [evidence TAS] [pmid 10207104] | |
| AC023992.8 | 17q23.2 | integral to membrane [goid | metal ion binding [goid 0046872]; protein | NA | NA |
| | | 0016021]; membrane [goid | binding [goid 0005515]; zinc ion binding | | |
| | | 0016020] | [goid 0008270] | | |
| AL359752.11 | 1p11-13.2 | NA | sugar binding [goid 0005529] [evidence | NA | NA |
| | | | IEA] | | |
| NM_080748.1 | 20q11.22 | integral to membrane [goid | NA | NA | NA |
| | | 0016021] [evidence IEA] | | | |
| NM_080748.1 | 20q11.22 | integral to membrane [goid | NA | NA | NA |
| | | 0016021] [evidence IEA] | | | |
| NM_138805.2 | 3p14.2 | extracellular region [goid | cytokine activity [goid 0005125] [evidence | negative regulation of insulin | NA |
| | | 0005576] [evidence NAS] | NAS] [pmid 12160727] | secretion [goid 0046676] [evidence | |
| | | [pmid 12160727] | | IDA] [pmid 12160727] | |
| NM_006418.3 | 13q14.3 | membrane [goid 0016020] | latrotoxin receptor activity [goid 0016524] | NA | NA |
| NM_024017.3 | 17q21.3 | nucleus [goid 0005634] | transcription factor activity [goid 0003700] | development [goid 0007275] | NA |
| | | [evidence NAS] | [evidence NAS]; transcriptional activator | [evidence NAS]; go_process: | |
| | | | activity [goid 0016563] [evidence IEA]; | regulation of transcription, DNA- | |
| | | | sequence-specific DNA binding [goid | dependent [goid 0006355] [evidence | |
| | | | 0043565] | NAS] | |
| NM_006149.2 | 19q13.2 | cytosol [goid 0005829] | sugar binding [goid 0005529] [evidence | cell adhesion [goid 0007155] | SB1a and CEA in the patches on |
| | | [evidence TAS] [pmid | TAS] [pmid 9162064] | [evidence TAS] [pmid 9162064] | the cell surface of human colon |
| | | 9162064]; plasma | | | adenocarcinoma cells could be |
| | | membrane [goid 0005886] | | | biologically important ligands for |
| | | [evidence TAS] [pmid | | | galectin-4 |
| | | 9162064] | | | |
| NM_001738.1; | 8q13-q22.1 | cytoplasm [goid 0005737] | lyase activity [goid 0016829] [evidence | one-carbon compound metabolism | NA |
| M33987.1 | | [evidence NR] | IEA]; zinc ion binding [goid 0008270] | [goid 0006730] [evidence IEA] | |
| | | | [evidence IEA]; carbonate dehydratase | | |
| | | | activity [goid 0004089] [evidence TAS] | | |
| | | | [pmid 2121614] | | |
| AY358469.1 | 1q44 | NA | NA | NA | NA |
| NM_017716.1 | 11q12 | integral to membrane [goid | receptor activity [goid 0004872] [evidence | signal transduction [goid 0007165] | NA |
| | | 0016021] [evidence IEA] | IEA] | [evidence IEA] | |
| NM_002644.2 | 1q31-q41 | integral to plasma | receptor activity [goid 0004872] [evidence | protein secretion [goid 0009306] | NA |
| | | membrane [goid 0005887] | IEA]; protein transporter activity [goid | [evidence NR] | |
| | | [evidence TAS] [pmid | 0008565] [evidence NR] | | |
| | | 2920039] | | | |
| NM_017625.2 | 1q21.3 | membrane [goid 0016020] | sugar binding [goid 0005529] [evidence | NA | Intelectin is consistently and |
| | | [evidence IEA] | IEA] | | highly overexpressed in a |
| | | | | | proportion of mesothelioma and |
| | | | | | gastrointestinal malignancies at |
| | | | | | the protein level |
| NM_031457.1 | 11q12.2 | integral to membrane [goid | receptor activity [goid 0004872] [evidence | signal transduction [goid 0007165] | |
| | | 0016021] [evidence IEA] | IEA] | [evidence IEA] | |
| NM_005727.2 | 1p34.1 | integral to membrane [goid | NA | cell adhesion [goid 0007155] | Overexpression of NET-1 is |
| | | 0016021] [evidence TAS] | | [evidence NR]; cell motility [goid | associated with undifferentiated |
| | | [pmid 9714763] | | 0006928] [evidence NR]; cell | squamous cell carcinoma of |
| | | | | proliferation [goid 0008283] | cervical neoplasms |
| | | | | [evidence NR] | |
| NM_003823.2 | 20q13.3 | soluble fraction [goid | receptor activity [goid 0004872] [evidence | apoptosis [goid 0006915] [evidence | DCR3 is located on 20q13; when |
| | | 0005625] [evidence TAS] | TAS] [pmid 9872321] | IEA]; anti-apoptosis [goid 0006916] | amplified in colorectal cancer, |
| | | [pmid 9872321] | | [evidence TAS] [pmid 9872321] | patients are less likely to respond |
| | | | | | to chemotherapy |
| NM_001415.2 | Xp22.2-p22.1 | eukaryotic translation | GTP binding [goid 0005525] [evidence | protein biosynthesis [goid 0006412] | NA |
| | | initiation factor 2 complex | IEA]; GTPase activity [goid 0003924] | [evidence IEA] | |
| | | [goid 0005850] [evidence | [evidence TAS] [pmid 8106381]; | | |
| | | NR]; cytosolic small | translation initiation factor activity [goid | | |
| | | ribosomal subunit (sensu | 0003743] [evidence IEA] | | |
| | | Eukaryota) [goid 0005843] | | | |
| | | [evidence NR] | | | |
| NM_012155.1 | 19q13.32 | microtubule associated | NA | visual perception [goid 0007601] | NA |
| | | complex [goid 0005875] | | [evidence TAS] [pmid 10521658]; | |
| | | [evidence TAS] [pmid | | perception of sound [goid 0007605] | |
| | | 10521658] | | [evidence TAS] [pmid 10521658] | |
| NM_000582.2 | 4q21-q25 | extracellular space [goid | protein binding [goid 0005515] [evidence | ossification [goid 0001503] [evidence | increased expression of the |
| | | 0005615] [evidence IEA]; | IEA]; integrin binding [goid 0005178] | IEA]; cell adhesion [goid 0007155] | alpha(v)beta(3) integrin during |
| | | extracellular matrix (sensu | [evidence NAS]; cytokine activity [goid | [evidence IEA]; anti-apoptosis [goid | breast cancer progression can |
| | | Metazoa) [goid 0005578] | 0005125] [evidence ISS]; growth factor | 0006916] [evidence ISS]; ossification | make tumor cells more |
| | | [evidence TAS] [pmid | activity [goid 0008083] [evidence TAS] | [goid 0001503] [evidence TAS] [pmid | responsive to malignancy- |
| | | 1107524] | [pmid 1107524] | 10766759]; cell-matrix adhesion | promoting ligands such as OPN |
| | | | | [goid 0007160] [evidence NAS]; cell- | and result in increased tumor cell |
| | | | | cell signaling [goid 0007267] | aggressiveness. |
| | | | | [evidence TAS] [pmid 1107524]; | |
| | | | | immune cell chemotaxis [goid | |
| | | | | 0030595] [evidence TAS] [pmid | |
| | | | | 1107524]; T-helper 1 type immune | |
| | | | | response [goid 0042088] [evidence | |
| | | | | TAS] [pmid 1107524]; induction of | |
| | | | | positive chemotaxis [goid 0050930] | |
| | | | | [evidence TAS] [pmid 1107524]; | |
| | | | | negative regulation of bone | |
| | | | | mineralization [goid 0030502] | |
| | | | | [evidence NAS] [pmid 1729712]; | |
| | | | | regulation of myeloid cell | |
| | | | | differentiation [goid 0045637] | |
| | | | | [evidence TAS] [pmid 1107524]; | |
| | | | | positive regulation of T cell | |
| | | | | proliferation [goid 0042102] | |
| | | | | [evidence TAS] [pmid 1107524] | |
| NM_032023.3 | 10q11.21 | NA | protein binding [goid 0005515] [evidence | signal transduction [goid 0007165] | NA |
| | | | IEA]; oxidoreductase activity [goid | [evidence IEA] | |
| | | | 0016491] [evidence IEA] | | |
| NM_144947.1 | 19q13.3-q13.4 | NA | trypsin activity [goid 0004295] [evidence | proteolysis and peptidolysis [goid | Kallikrein 11 is an independent |
| | | | IEA]; chymotrypsin activity [goid 0004263] | 0006508] [evidence IEA] | marker of favorable prognosis in |
| | | | [evidence IEA] | | ovarian cancer patients. |
| AC084847.5 | 8p12 | NA | NA | NA | NA |
| NM_017763.3; | 17q23.2 | ubiquitin ligase complex | zinc ion binding [goid 0008270] [evidence | protein ubiquitination [goid 0016567] | |
| AB081837.1 | | [goid 0000151] [evidence | IEA]; ubiquitin-protein ligase activity [goid | [evidence IEA] | |
| | | IEA] | 0004842] [evidence IEA] | | |
| AJ236922.1 | 7q31-3-q32.1 | membrane [goid 0016020] | receptor activity [goid 0004872] [evidence | sensory perception [goid 0007600] | NA |
| | | [evidence IEA]; integral to | IEA]; metabotropic glutamate, GABA-B- | [evidence IEA]; perception of smell | |
| | | plasma membrane [goid | like receptor activity [goid 0008067] | [goid 0007608] [evidence IEA]; | |
| | | 0005887] [evidence TAS] | [evidence IEA]; metabotropic glutamate, | signal transduction [goid 0007165] | |
| | | [pmid 9473604] | GABA-B-like receptor activity [goid | [evidence IEA]; synaptic | |
| | | | 0008067] [evidence TAS] [pmid 9473604] | transmission [goid 0007268] | |
| | | | | [evidence NR]; visual perception | |
| | | | | [goid 0007601] [evidence TAS] [pmid | |
| | | | | 9473604]; G-protein coupled | |
| | | | | receptor protein signaling pathway | |
| | | | | [goid 0007186] [evidence IEA]; | |
| | | | | negative regulation of adenylate | |
| | | | | cyclase activity [goid 0007194] | |
| | | | | [evidence TAS] [pmid 9473604] | |
| NM_002483.3 | 19q13.2 | membrane [goid 0016020] | NA | cell-cell signaling [goid 0007267] | Levels of CEACAM6 expression |
| | | [evidence IEA]; integral to | | [evidence TAS] [pmid 3220478]; | can modulate pancreatic |
| | | plasma membrane [goid | | signal transduction [goid 0007165] | adenocarcinoma cellular |
| | | 0005887] [evidence TAS] | | [evidence TAS] [pmid 3220478] | invasiveness in a c-Src- |
| | | [pmid 3220478] | | | dependent manner |
| NM_006408.2 | 7p21.3 | GO: 0005615: extracellular | NA | NA | Differentiation, associated with |
| | | space [evidence TAS] | | | ER positive tumors and interacts |
| | | | | | with metastasis genes; A |
| | | | | | prognostic effect of AGR2 for |
| | | | | | overall survival could be shown, |
| | | | | | which became independently |
| | | | | | significant for the group of nodal- |
| | | | | | negative tumors |
| NM_004864.1 | 19p13.1-13.2 | GO: 0005576: extracellular | GO: 0005125: cytokine activity; | GO: 0007267: cell-cell signaling; | Microarray analysis identifies |
| | | region | GO: 0008083: growth factor activity | GO: 0007165: signal transduction; | MIC-1 as being upregulated in |
| | | | | GO: 0007179: transforming growth | cancer of breast, prostate, and |
| | | | | factor beta receptor signaling | colon. Tissues from these |
| | | | | pathway | patients show increased MIC-1 |
| | | | | | by IHC and their serum shows |
| | | | | | elevated levels. |
| NM_012445.1 | 4p16.3 | GO: 0005615: extracellular | GO: 0005515: protein binding | GO: 0007275: development; | SPON2/Mindin is differentially |
| | | space; GO: 0005578: | | GO: 0006955: immune response; | expressed in cancer versus |
| | | extracellular matrix | | GO: 0007411: axon guidance | normal tissue |
| | | | | [evidence TAS] [pmid 10512675]; | |
| | | | | GO: 0006935: chemotaxis; | |
| | | | | GO: 0030335: positive regulation of | |
| | | | | cell migration; GO: 0001569: | |
| | | | | patterning of blood vessels; | |
| | | | | GO: 0045766: positive regulation of | |
| | | | | angiogenesis; GO: 0007155: cell | |
| | | | | adhesion | |
| NM_138938.1 | 2p12 | cytoplasm [goid 0005737] | sugar binding [goid 0005529] [evidence | development [goid 0007275] | |
| | | [evidence TAS] [pmid | TAS] [pmid 1325291] | [evidence TAS] [pmid 8997243]; | |
| | | 8997243]; soluble fraction | | acute-phase response [goid | |
| | | [goid 0005625] [evidence | | 0006953] [evidence IEA]; | |
| | | TAS] [pmid 1325291]; | | inflammatory response [goid | |
| | | extracellular space [goid | | 0006954] [evidence IEA]; cell | |
| | | 0005615] [evidence TAS] | | proliferation [goid 0008283] | |
| | | [pmid 8997243] | | [evidence TAS] [pmid 8997243]; | |
| | | | | heterophilic cell adhesion [goid | |
| | | | | 0007157] [evidence TAS] [pmid | |
| | | | | 8997243] | |
| BC070213.1 | 1q23.2 | membrane [goid 0016020] | NA | NA | NA |
| | | [evidence IEA]; integral to | | | |
| | | plasma membrane [goid | | | |
| | | 0005887] [evidence IEA] | | | |
| NM_006475.1 | 13q13.3 | extracellular matrix (sensu | heparin binding [goid 0008201] [evidence | cell adhesion [goid 0007155] | Data suggest that periostin- |
| | | Metazoa) [goid 0005578] | ISS]; protein binding [goid 0005515] | [evidence IEA]; cell adhesion [goid | mediated angiogenesis derives in |
| | | [evidence IEA]; extracellular | [evidence IEA] | 0007155] [evidence IDA] [pmid | part from the up-regulation of the |
| | | matrix (sensu Metazoa) | | 12235007]; skeletal development | vascular endothelial growth factor |
| | | [goid 0005578] [evidence | | [goid 0001501] [evidence TAS] [pmid | receptor Flk-1/KDR by |
| | | ISS] | | 8363580] | endothelial cells through an |
| | | | | | integrin alpha(v)beta(3)-focal |
| | | | | | adhesion kinase signaling |
| | | | | | pathway. Over expression of |
| | | | | | Periostin promotes metastatic |
| | | | | | growth of colon cancer by |
| | | | | | augmenting cell survival via the |
| | | | | | Akt/PKB pathway |
| NM_004385.2 | 5q14.3 | GO: 0005578: extracellular | GO: 0005529: sugar binding; GO: 0005540: | GO: 0008037: cell recognition; | involved in the progression of |
| | | matrix | hyaluronic acid binding; GO: 0005509: | GO: 0007275: development | melanomas and may be a |
| | | | calcium ion binding | | reliable marker for clinical |
| | | | | | diagnosis |
| NM_004385.2 | 5q14.3 | GO: 0005578: extracellular | GO: 0005529: sugar binding; GO: 0005540: | GO: 0008037: cell recognition; | involved in the progression of |
| | | matrix | hyaluronic acid binding; GO: 0005509: | GO: 0007275: development | melanomas and may be a |
| | | | calcium ion binding | | reliable marker for clinical |
| | | | | | diagnosis |
| BC021275.2 | 5q23-5q31 | endoplasmic reticulum [goid | NA | NA | NA |
| | | 0005783] | | | |
| NM_005408.2 | 17q11.2 | membrane [goid 0016020] | chemokine activity [goid 0008009] | chemotaxis [goid 0006935] | NA |
| | | [evidence IEA]; extracellular | [evidence TAS] [pmid 9558100]; | [evidence TAS] [pmid 9195948]; | |
| | | space [goid 0005615] | chemokine receptor activity [goid | sensory perception [goid 0007600] | |
| | | [evidence TAS] [pmid | 0004950] [evidence NR] | [evidence IEA]; cell-cell signaling | |
| | | 9195948] | | [goid 0007267] [evidence TAS] [pmid | |
| | | | | 9195948]; signal transduction [goid | |
| | | | | 0007165] [evidence TAS] [pmid | |
| | | | | 9195948]; signal transduction [goid | |
| | | | | 0007165] [evidence TAS] [pmid | |
| | | | | 9558100]; inflammatory response | |
| | | | | [goid 0006954] [evidence TAS] [pmid | |
| | | | | 9195948]; calcium ion homeostasis | |
| | | | | [goid 0006874] [evidence TAS] [pmid | |
| | | | | 9195948] | |
| NM_018098.4 | 3q26.1-q26.2 | GO: 0005622: intracellular | GO: 0005085: guanyl-nucleotide | GO: 0007242: intracellular signaling | XRCC1, CLB6, and BRCT |
| | | | exchange factor activity; GO: 0004871: | cascade; GO: 0043123: positive | domains of ECT2 play a critical |
| | | | signal transducer activity | regulation of I-kappaB kinase/NF- | role in regulating cytokinesis |
| | | | | kappaB cascade | |
| NM_006645.1 | 11q13 | NA | NA | NA | Scanlan, M. J., Chen, Y. T., |
| | | | | | Williamson, B., Gure, A. O., |
| | | | | | Stockert, E., Gordan, J. D., |
| | | | | | Tureci, O., Sahin, U., |
| | | | | | Pfreundschuh, M. and Old, L. J. |
| | | | | | Characterization of human colon |
| | | | | | cancer antigens recognized by |
| | | | | | autologous antibodies Int. J. |
| | | | | | Cancer 76 (5), 652-658 (1998) |
| NM_004625.3 | 3p25 | GO: 0005576: extracellular | GO: 0005102: receptor binding [evidence | GO: 0007275: development[evidence | Expression inversely associated |
| | | [evidence IEA]; | NAS] [pmid 8893824]; GO: 0004871: | IEA]; GO: 0009653: morphogenesis | to ER in uterine leyoma |
| | | GO: 0005615: extracellular | signal transducer activity [evidence IEA] | [evidence TAS] [pmid 9161407]; | |
| | | space [evidence NR] | | GO: 0007267: cell-cell signaling | |
| | | | | [evidence NR]; GO: 0007548: sex | |
| | | | | differentiation [evidence TAS] [pmid | |
| | | | | 9790192]; GO: 0007165: signal | |
| | | | | transduction [evidence NAS] [pmid | |
| | | | | 8893824]; GO: 0007223: frizzled-2 | |
| | | | | signaling pathway [evidence IEA] | |
| NM_001008540.1 | 2q21 | GO: 0016021: integral to | GO: 0016493: C-C chemokine receptor | GO: 0007186: G-protein coupled | CXCR4 is induced by NF-kappa |
| | | membrane [evidence IEA] | activity [evidence IEA]; GO: 0001584: | receptor protein signaling pathway | B and has a role in breast cancer |
| | | | rhodopsin-like receptor activity [evidence | [evidence IEA] | cell migration and metastasis. |
| | | | IEA]; GO: 0016494: C—X—C chemokine | | |
| | | | receptor activity [evidence NAS] [pmid | | |
| | | | 9468539] | | |
| NM_000579.1 | 3p21 | GO: 0016021: integral to | GO: 0004872: receptor activity [evidence | GO: 0007186: G-protein coupled | CCR5 activity influences human |
| | (LOH) | membrane [evidence IEA] | IEA]; GO: 0016493: C-C chemokine | receptor protein signaling pathway | breast cancer progression in a |
| | | | receptor activity [evidence IEA]; | [evidence IEA] | p53-dependent manner |
| | | | GO: 0001584: rhodopsin-like receptor | | |
| | | | activity [evidence IEA] | | |
| NM_004367.3 | 6q27 | GO: 0005887: integral to | GO: 0016493: C-C chemokine receptor | GO: 0007186: G-protein coupled | CCR6 on polarized intestinal |
| | | plasma membrane | activity [evidence IEA]; GO: 0004872: | receptor protein signaling pathway | epithelial cells, alter specialized |
| | | [evidence TAS] [PMID: | receptor activity [evidence TAS] [PMID: | [evidence IEA]; GO: 0019735: | intestinal epithelial cell functions, |
| | | 9186513] | 9186513]; GO: 0001584: rhodopsin-like | antimicrobial humoral response | including electrogenic ion |
| | | | receptor activity [evidence IEA] | (sensu Vertebrata) [evidence TAS] | secretion and possibly epithelial |
| | | | | [PMID: 9186513]; GO: 0006928: cell | cell adhesion and migration |
| | | | | motility [evidence TAS] [PMID: | |
| | | | | 9186513]; GO: 0006968: cellular | |
| | | | | defense response [evidence TAS] | |
| | | | | [PMID: 10521347]; GO: 0006935: | |
| | | | | chemotaxis [evidence TAS] [PMID: | |
| | | | | 11001880]; GO: 0006959: humoral | |
| | | | | immune response [evidence TAS] | |
| | | | | [PMID: 11001880]; GO: 0007204: | |
| | | | | positive regulation of cytosolic | |
| | | | | calcium ion concentration [evidence | |
| | | | | TAS] [PMID: 9223454]; | |
| | | | | GO: 0007165: signal transduction | |
| | | | | [evidence TAS] [PMID: 9186513] | |
| NM_004591.1 | 2q33-q37 | GO: 0005615: extracellular | GO: 0008009: chemokine activity | GO: 0019735: antimicrobial humoral | Results describe the relationship |
| | | space [evidence TAS] [pmid | [evidence TAS] [pmid 10438902]; | response (sensu Vertebrata) | between cancer-related factors |
| | | 9038201]; | | [evidence TAS] [pmid 9038201]; | and serum levels of macrophage |
| | | | | GO: 0007267: cell-cell signaling | inflammatory protein-3alpha in |
| | | | | [evidence TAS] [pmid 9038201]; | hepatocellular carcinoma. |
| | | | | GO: 0006935: chemotaxis [evidence | |
| | | | | TAS] [pmid 10438902]; | |
| | | | | GO: 0006954: inflammatory response | |
| | | | | [evidence TAS] [pmid 9129037]; | |
| | | | | GO: 0007165: signal transduction | |
| | | | | [evidence TAS] [pmid 9038201] | |
| NM_006564.1 | 3p21.31 | GO: 0005887: integral to | GO: 0016493: C-C chemokine receptor | GO: 0007186: G-protein coupled | NA |
| | | plasma membrane | activity [evidence IEA]; GO: 0016494: | receptor protein signaling pathway | |
| | | [evidence TAS] [pmid | C—X—C chemokine receptor activity | [evidence TAS] [pmid 9166430]; | |
| | | 9166430] | [evidence IEA]; GO: 0015026: coreceptor | GO: 0019079: viral genome | |
| | | | activity [evidence TAS] [pmid 9166430]; | replication [evidence TAS] [pmid | |
| | | | GO: 0001584: rhodopsin-like receptor | 9230441] | |
| | | | activity [evidence IEA]; | | |
| NM_178445.1 | 3q22.1 | GO: 0005887: integral to | GO: 0016493: C-C chemokine receptor | GO: 0007186: G-protein coupled | NA |
| | | plasma membrane | activity [evidence IEA]; GO: 0001584: | receptor protein signaling pathway | |
| | | [evidence TAS] [PMID: | rhodopsin-like receptor activity [evidence | [evidence TAS] [PMID: 10734104]; | |
| | | 10767544] | IEA] | GO: 0006935: chemotaxis [evidence | |
| | | | | TAS] [PMID: 10706668]; | |
| | | | | GO: 0006955: immune response | |
| | | | | [evidence TAS] [PMID: 10706668] | |
| NM_003965.3 | 3p21.31 | GO: 0016021: integral to | GO: 0016493: C-C chemokine receptor | GO: 0007186: G-protein coupled | NA |
| | | membrane [evidence IEA]; | activity [evidence IEA]; GO: 0004872: | receptor protein signaling pathway | |
| | | GO: 0005887: integral to | receptor activity [evidence IEA]; | [evidence IEA] [evidence TAS] | |
| | | plasma membrane | GO: 0001584: rhodopsin-like receptor | [PMID: 9473515]; GO: 0019735: | |
| | | [evidence TAS] [PMID: | activity [evidence IEA] | antimicrobial humoral response | |
| | | 9473515] | | (sensu Vertebrata) [evidence TAS] | |
| | | | | [PMID: 9473515]; GO: 0006935: | |
| | | | | chemotaxis [evidence TAS] [PMID: | |
| | | | | 9473515] | |
| NM_001838.2 | 17q12-q21.2 | integral to plasma | C-C chemokine receptor activity [goid | G-protein coupled receptor protein | Overexpression of CCR7 mRNA |
| | (amp) | membrane [goid 0005887]; | 0016493]; receptor activity [goid | signaling pathway [goid 0007186]; | in nonsmall cell lung cancer is |
| | | plasma membrane [goid | 0004872]; rhodopsin-like receptor activity | chemotaxis [goid 0006935]; | associated with development of |
| | | 0005886] | [goid 0001584] | elevation of cytosolic calcium ion | lymph node metastasis |
| | | | | concentration [goid 0007204]; | |
| | | | | inflammatory response [goid | |
| | | | | 0006954]; signal transduction [goid | |
| | | | | 0007165] | |
| NM_002989.2 | 9p13.3 | extracellular region [goid | chemokine activity [goid 0008009; | cell-cell signaling [goid 0007267]; | Cathepsin D specifically cleaves |
| | | 0005576]; extracellular | evidence IEA, TAS] | chemotaxis [goid 0006935]; signal | this protein that is expressed in |
| | | space [goid 0005615] | | transduction [goid 0007165] | human breast cancer. |
| NM_001554.3 | 1p22.3 | GO: 0005576: extracellular | GO: 0008201: heparin binding; | GO: 0006935: chemotaxis; | promotes tumor growth; |
| | | | GO: 0005520: insulin-like growth factor | GO: 0007155: cell adhesion; | increased Cyr61 expression is |
| | | | binding | GO: 0009653: morphogenesis [pmid | associated with an aggressive |
| | | | | 9135077]; GO: 0008283: cell | phenotype of breast cancer cells |
| | | | | proliferation [pmid 9135077]; | |
| | | | | GO: 0001558: regulation of cell | |
| | | | | growth | |
| AY327584.1 | 1q21 | Cytoskeleton [goid | actin binding [goid 0003779]; hormone | NA | NA |
| | | 0005856]; extracellular | activity [goid 0005179] | | |
| | | region [goid 0005576]; | | | |
| | | integral to plasma | | | |
| | | membrane [goid 0005887] | | | |
| NM_006988.3 | 21q21.2 | GO: 0005578: extracellular | GO: 0008201: heparin binding [evidence | GO: 0007229: integrin-mediated | This gene encodes a disintegrin |
| | | matrix (sensu Metazoa) | IEA]; GO: 0016787; hydrolase activity | signaling pathway [evidence TAS] | and metalloproteinase with |
| | | [evidence IEA] | [evidence IEA]; GO: 0005178: integrin | [pmid 8995297]; GO: 0006508: | thrombospondin motifs-1 |
| | | | binding [evidence NR]; GO: 0004222: | proteolysis and peptidolysis | (ADAMTS1), which is a member |
| | | | metalloendopeptidase activity [evidence | [evidence IEA]; GO: 0008285: | of the ADAMTS protein family. |
| | | | IEA]; GO: 0008270: zinc ion binding | negative regulation of cell | Members of the family share |
| | | | [evidence IEA] | proliferation [evidence TAS] [pmid | several distinct protein modules, |
| | | | | 10438512] | including a propeptide region, a |
| | | | | | metalloproteinase domain, a |
| | | | | | disintegrin-like domain, and a |
| | | | | | thrombospondin type 1 (TS) |
| | | | | | motif. Individual members of this |
| | | | | | family differ in the number of C- |
| | | | | | terminal TS motifs, and some |
| | | | | | have unique C-terminal domains. |
| | | | | | The protein encoded by this gene |
| | | | | | contains 2 disintegrin loops and 3 |
| | | | | | C-terminal TS motifs and has |
| | | | | | anti-angiogenic activity. The |
| | | | | | expression of this gene may be |
| | | | | | associated with various |
| | | | | | inflammatory processes as well |
| | | | | | as development of cancer |
| | | | | | cachexia. This gene is likely to be |
| | | | | | necessary for normal growth, |
| | | | | | fertility, and organ morphology |
| | | | | | and function. |
| NM_001571.2 | 19q13.3-q13.4 | GO: 0005634: nucleus | GO: 0003702: RNA polymerase II | GO: 0006355: regulation of | hIRF3 inhibited cell growth, |
| | | [evidence IEA] | transcription factor activity [evidence TAS] | transcription, DNA-dependent | blocked DNA synthesis, and |
| | | | [PMID: 8524823]; GO: 0003712: | [evidence IEA]; GO: 0006350: | induced apoptosis, while a |
| | | | transcription cofactor activity [evidence | transcription [evidence IEA]; | dominant negative mutant |
| | | | TAS] [PMID: 8524823]; GO: 0003700: | GO: 0006366: transcription from Pol | transformed 3T3 cells, implying |
| | | | transcription factor activity [evidence IEA] | II promoter [evidence TAS] [PMID: | that IRF3 may function as a |
| | | | | 8524823] | tumor suppressor and its |
| | | | | | dominant negative mutant may |
| | | | | | have a role in tumorigenesis. |
| NM_145306.1 | 10q22.1 | integral to plasma | protein binding [goid 0005515] | NA | NA |
| | | membrane [goid 0005887] | | | |
| BC042754.1 | 11p13 | NA | receptor activity [goid 0004872] | NA | NA |
| NM_001908.3 | 8p22 | lysosome [goid 0005764] | cathepsin B activity [goid 0004213] | proteolysis [goid 0006508] [evidence | Secreted |
| | | [evidence IEA]; intracellular | [evidence TAS] [pmid 1645961] | TAS] [pmid 3463996] | |
| | | [goid 0005622] [evidence | | | |
| | | TAS] [pmid 1645961] | | | |
| NM_031419.2 | 3p12-q12 | NA | NA | NA | lkappaB-zeta harbors latent |
| | | | | | transcriptional activation activity |
| | | | | | which is expressed upon |
| | | | | | interaction with the NF-kappaB |
| | | | | | p50 subunit |
| NM_006096.2 | 8q24.3 | nucleus [goid 0005634] | catalytic activity [goid 0003824] [evidence | cell differentiation [goid 0030154] | Drg1 expression may be |
| | | [evidence IEA] | IEA] | [evidence IEA]; response to metal | associated with a less |
| | | | | ion [goid 0010038] [evidence TAS] | aggressive, indolent colorectal |
| | | | | [pmid 9605764] | cancer. |
| NM_006096.2 | 8q24.3 | nucleus [goid 0005634] | catalytic activity [goid 0003824] [evidence | cell differentiation [goid 0030154] | Drg1 expression may be |
| | | [evidence IEA] | IEA] | [evidence IEA]; response to metal | associated with a less |
| | | | | ion [goid 0010038] [evidence TAS] | aggressive, indolent colorectal |
| | | | | [pmid 9605764] | cancer. |
| NM_2075201 | 2p16.3 | integral to membrane [goid | protein binding [goid 0005515] [evidence | regulation of apoptosis [goid | ASY may be multi-functional, |
| | | 0016021] [evidence IEA]; | IPI] [pmid 11126360] | 0042981] [evidence NAS] [pmid | regulating apoptosis, tumor |
| | | nuclear membrane [goid | | 11126360]; negative regulation of | development, and neuronal |
| | | 0005635] [evidence IDA] | | anti-apoptosis [goid 0019987] | regeneration [review] |
| | | [pmid 11126360]; | | [evidence IMP] [pmid 11126360]; | |
| | | endoplasmic reticulum [goid | | negative regulation of axon | |
| | | 0005783] [evidence IEA]; | | extension [goid 0030517] [evidence | |
| | | endoplasmic reticulum [goid | | IDA] [pmid 10667797] | |
| | | 0005783] [evidence NAS] | | | |
| | | [pmid 11126360]; integral to | | | |
| | | endoplasmic reticulum | | | |
| | | membrane [goid 0030176] | | | |
| | | [evidence IEP] [pmid | | | |
| | | 10667797] | | | |
| NM_005063.4 | 10q23-q24 | membrane [goid 0016020] | iron ion binding [goid 0005506] [evidence | fatty acid biosynthesis [goid | loss of SCD expression is a |
| | | [evidence IEA]; integral to | IEA]; oxidoreductase activity [goid | 0006633] [evidence IEA] | frequent event in prostate |
| | | membrane [goid 0016021] | 0016491] [evidence IEA]; stearoyl-CoA 9- | | adenocarcinoma |
| | | [evidence IEA]; | desaturase activity [goid 0004768] | | |
| | | endoplasmic reticulum [goid | [evidence TAS] [pmid 10229681] | | |
| | | 0005783] [evidence IEA] | | | |
| NM_198976.1 | 20q13.32 | nucleus [goid 0005634] | protein binding [goid 0005515] [evidence | transcription [goid 0006350] | NA |
| | | [evidence IEA] | IPI] [pmid 12620389] | [evidence IEA]; negative regulation | |
| | | | | of transcription [goid 0016481] | |
| | | | | [evidence IEA]; regulation of | |
| | | | | transcription, DNA-dependent [goid | |
| | | | | 0006355] [evidence IEA] | |
| CR749471.1 | 9q32 | Nucleus [goid 0005634] | RNA binding [goid 0003723]; nucleic acid | RNA splicing [goid 0008380]; | NA |
| | | | binding [goid 0003676]; nucleotide | anatomical structure morphogenesis | |
| | | | binding [goid 0000166] | [goid 0009653]; mRNA processing | |
| | | | | [goid 0006397] | |
| AC021236.10 | 8q11.21 | NA | NA | NA | NA |
| NM_024918.2 | 20q11.23 | nucleus [goid 0005634] | NA | NA | NA |
| | | [evidence IEA] | | | |
| AC093619.5 | 7q22.1 | NA | NA | NA | NA |
| NM_005564.2 | 9q34.11 | cytoplasm [goid 0005737] | binding [goid 0005488] [evidence IEA]; | transport [goid 0006810] [evidence | These data characterize lipocalin |
| | | [evidence NR]; soluble | transporter activity [goid 0005215] | IEA] | 2 as an epithelial inducer in Ras |
| | | fraction [goid 0005625] | [evidence IEA] | | malignancy and a suppressor of |
| | | [evidence NR] | | | metastasis. |
| AY623117.1 | 1p33 | GO: 0005634: nucleus [TAS] | GO: 0005524: ATP binding [IEA]; | GO: 0007126: meiosis [TAS]; | The protein encoded by this gene |
| | (LOH) | | GO: 0003677: DNA binding [IEA]; | GO: 0006281: DNA repair [TAS]; | belongs to the DEAD-like |
| | | | GO: 0004386: helicase activity [IEA]; | GO: 0006310: DNA recombination | helicase superfamily, and shares |
| | | | GO: 0016787: hydrolase activity [IEA] | [TAS];: GO: 0008151: cell growth | similarity with Saccharomyces |
| | | | | and/or maintenance [IEA] | cerevisiae Rad54, a protein |
| | | | | | known to be involved in the |
| | | | | | homologous recombination and |
| | | | | | repair of DNA. This protein has |
| | | | | | been shown to play a role in |
| | | | | | homologous recombination |
| | | | | | related repair of DNA double- |
| | | | | | strand breaks. The binding of this |
| | | | | | protein to double-strand DNA |
| | | | | | induces a DNA topological |
| | | | | | change, which is thought to |
| | | | | | facilitate homologous DNA |
| | | | | | paring, and stimulate DNA |
| | | | | | recombination. |
| NM_005201.2 | 3p22 | GO: 0005887: integral to | GO: 0015026: coreceptor activity | GO: 0006935: chemotaxis [evidence | This gene encodes a member of |
| | (amp) | plasma membrane | [evidence TAS] [pmid 9417093]; | TAS] [pmid 10910894]; | the beta chemokine receptor |
| | | | GO: 0016493: C-C chemokine receptor | GO: 0007155: cell adhesion | family, which is predicted to be a |
| | | | activity [evidence IEA]; GO: 0001584: | [evidence TAS] [pmid 10910894]; | seven transmembrane protein |
| | | | rhodopsin-like receptor activity [evidence | GO: 0006955: immune response | similar to G protein-coupled |
| | | | IEA]; | [evidence TAS] [pmid 9670926]; | receptors. Chemokines and their |
| | | | | GO: 0007204: cytosolic calcium ion | receptors are important for the |
| | | | | concentration elevation [evidence | migration of various cell types |
| | | | | TAS] [pmid 9417093]; GO: 0007186: | into the inflammatory sites. This |
| | | | | G-protein coupled receptor protein | receptor protein preferentially |
| | | | | signaling pathway [evidence TAS] | expresses in the thymus. I-309, |
| | | | | [pmid 8816377] | thymus activation-regulated |
| | | | | | cytokine (TARC) and |
| | | | | | macrophage inflammatory |
| | | | | | protein-1 beta (MIP-1 beta) have |
| | | | | | been identified as ligands of this |
| | | | | | receptor. Studies of this receptor |
| | | | | | and its ligands suggested its role |
| | | | | | in regulation of monocyte |
| | | | | | chemotaxis and thymic cell |
| | | | | | apoptosis. More specifically, this |
| | | | | | receptor may contribute to the |
| | | | | | proper positioning of activated T |
| | | | | | cells within the antigenic |
| | | | | | challenge sites and specialized |
| | | | | | areas of lymphoid tissues. This |
| | | | | | gene is located at the chemokine |
| | | | | | receptor gene cluster region. |
| NM_139276.2 | 17q21.31 | nucleus [goid 0005634] | calcium ion binding [goid 0005509] | cell motility [goid 0006928] [evidence | TFF3 and the essential tumor |
| | | [evidence IEA]; nucleus | [evidence IEA]; signal transducer activity | TAS] [pmid 9670957]; acute-phase | angiogenesis regulator |
| | | [goid 0005634] [evidence | [goid 0004871] [evidence IEA]; | response [goid 0006953] [evidence | VEGF(165) exert potent |
| | | TAS] [pmid 7512451]; | transcription factor activity [goid 0003700] | NR]; JAK-STAT cascade [goid | proinvasive activity through |
| | | cytoplasm [goid 0005737] | [evidence IEA]; transcription factor | 0007259] [evidence TAS] [pmid | STAT3 signaling in human |
| | | [evidence TAS] [pmid | binding [goid 0008134] [evidence IPI] | 15664994]; nervous system | colorectal cancer cells. |
| | | 7512451] | [pmid 15664994]; transcription factor | development [goid 0007399] | |
| | | | activity [goid 0003700] [evidence TAS] | [evidence TAS] [pmid 10205054]; | |
| | | | [pmid 7512451]; transcription factor | intracellular signaling cascade [goid | |
| | | | activity [goid 0003700] [evidence TAS] | 0007242] [evidence IEA]; regulation | |
| | | | [pmid 8675499]; hematopoietin/interferon- | of transcription, DNA-dependent | |
| | | | class (D200-domain) cytokine receptor | [goid 0006355] [evidence IEA]; | |
| | | | signal transducer activity [goid 0005062] | cytokine and chemokine mediated | |
| | | | [evidence TAS] [pmid 7512451] | signaling pathway [goid 0019221] | |
| | | | | [evidence NAS] [pmid 15664994]; | |
| | | | | negative regulation of transcription | |
| | | | | from RNA polymerase II promoter | |
| | | | | [goid 0000122] [evidence TAS] [pmid | |
| | | | | 8675499] | |
| NM_004994.1 | 20q11.2-q13.1 | GO: 0005615: extracellular | GO: 0016787: hydrolase activity [evidence | GO: 0030574: collagen catabolism | |
| | | space [evidence TAS] [pmid | IEA]; GO: 0008270: zinc ion binding | [evidence IEA] | |
| | | 2551898]; GO: 0005578: | [evidence TAS] [pmid 2551898]; | | |
| | | extracellular matrix (sensu | GO: 0004229: gelatinase B activity | | |
| | | Metazoa) [evidence IEA] | [evidence IEA]; GO: 0008133: collagenase | | |
| | | | activity [evidence TAS] [pmid 2551898] | | |
| NM_003219.1 | 5p15.33 | GO: 0005634: nucleus | GO: 0003677: DNA binding [evidence | GO: 0006278: RNA-dependent DNA | hTERT is transcriptionally |
| | (amp) | [evidence IEA]; | IEA]; GO: 0003723: RNA binding | replication [evidence IEA]; | regulated by raloxifene via an |
| | | GO: 0000781: chromosome, | [evidence IEA] GO: 0016740: transferase | GO: 0007004: telomerase-dependent | estrogen-responsive element- |
| | | telomeric region [evidence | activity [evidence IEA]; GO: 0042162: | telomere maintenance [evidence | dependent mechanism, which |
| | | IC] [pmid 12135483]; | telomeric DNA binding [evidence TAS] | IEA] | inhibits E2-induced up- regulation |
| | | GO: 0005697: telomerase | [pmid 9288757]; GO: 0003964: RNA- | | of telomerase activity. |
| | | holoenzyme complex | directed DNA polymerase activity | | Telomerase activity in |
| | | [evidence IDA] [pmid | [evidence IEA]; GO: 0003721: telomeric | | microdissected human breast |
| | | 12135483] | template RNA reverse transcriptase | | cancer tissues: association with |
| | | | activity [evidence IEA] [evidence TAS] | | p53, p21 and outcome. |
| | | | [pmid 14991929] | | |
| NM_001071.1 | 18p11.32 | | transferase activity [goid 0016740] | DNA repair [goid 0006281] [evidence | TS and DPD quantitation may be |
| | | | [evidence IEA]; methyltransferase activity | NAS] [pmid 15504738]; dTMP | helpful to evaluate prognosis of |
| | | | [goid 0008168] [evidence IEA]; | biosynthesis [goid 0006231] | patients receiving adjuvant 5-FU |
| | | | thymidylate synthase activity [goid | [evidence IEA]; DNA replication [goid | and that patients with high TS |
| | | | 0004799] [evidence IEA] | 0006260] [evidence NAS] [pmid | and low DPD may benefit from |
| | | | | 15504738]; nucleotide biosynthesis | adjuvant 5-FU chemotherapy in |
| | | | | [goid 0009165] [evidence IEA]; | colorectal cancer. |
| | | | | phosphoinositide-mediated signaling | |
| | | | | [goid 0048015] [evidence NAS] | |
| | | | | [pmid 15504738]; | |
| | | | | deoxyribonucleoside | |
| | | | | monophosphate biosynthesis [goid | |
| | | | | 0009157] [evidence TAS] [pmid | |
| | | | | 2987839]; nucleobase, nucleoside, | |
| | | | | nucleotide and nucleic acid | |
| | | | | metabolism [goid 0006139] | |
| | | | | [evidence TAS] [pmid 2987839] | |
| NM_198496.1 | 10q25.3 | NA | calcium ion binding [goid 0005509] | NA | CCSP-2 is a novel candidate for |
| | | | [evidence IEA] | | development as a diagnostic |
| | | | | | serum marker of early stage |
| | | | | | colon cancer |
| NM_199168.1 | 10q11.1 | GO: 0005576: extracellular | GO: 0008009: chemokine activity | GO: 0007186: G-protein coupled | SDF-1alpha and its receptor |
| | | region [evidence IEA] | [evidence TAS] [pmid 10772939]; | receptor protein signaling pathway | chemokine receptor CXCR4 |
| | | | GO: 0008083: growth factor activity | [evidence TAS] [pmid 8752280]; | induced transendothelial breast |
| | | | [evidence IEA] | GO: 0006874: calcium ion | cancer cell migration through |
| | | | | homeostasis [evidence TAS] [pmid | activation of the PI-3K/AKT |
| | | | | 10772939]; GO: 0007155: cell | pathway and Ca(2+)-mediated |
| | | | | adhesion [evidence TAS] [pmid | signaling. |
| | | | | 10198043]; GO: 0007267: cell-cell | |
| | | | | signaling [evidence NR]; | |
| | | | | GO: 0006935: chemotaxis [evidence | |
| | | | | TAS] [pmid 10620615]; | |
| | | | | GO: 0008015: circulation [evidence | |
| | | | | TAS] [pmid 10772939]; | |
| | | | | GO: 0006954: inflammatory response | |
| | | | | [evidence NR]; GO: 0008064: | |
| | | | | regulation of actin polymerization | |
| | | | | and/or depolymerization [evidence | |
| | | | | TAS] [pmid 10570282]; | |
| | | | | GO: 0009615: response to virus | |
| | | | | [evidence TAS] [pmid 10772939]; | |
| | | | | GO: 0007165: signal transduction | |
| | | | | [evidence TAS] [pmid 10491003] | |
| NM_022059.1 | 17p13 | GO: 0005576: extracellular | GO: 0005125: cytokine activity [evidence | GO: 0006935: chemotaxis [evidence | NA |
| | (LOH) | region [evidence NAS] | IEA]; GO: 0005044: scavenger receptor | NAS] [PMID: 11290797]; | |
| | | [PMID: 11017100]; | activity [evidence TAS] [PMID: 11060282] | GO: 0048247: lymphocyte | |
| | | GO: 0016021: integral to | | chemotaxis [evidence NAS] [PMID: | |
| | | membrane [evidence NAS] | | 11017100]; GO: 0006898: receptor | |
| | | [PMID: 11017100] [PMID: | | mediated endocytosis [evidence | |
| | | 11290797] | | NAS] [PMID: 11060282] | |
| NM_003376.3 | 6p12 | GO: 0016020: membrane | GO: 0008201: heparin binding [evidence | GO: 0001525: angiogenesis | During tumor progression there is |
| | | [evidence IEA]; | IEA]; [evidence IDA] [pmid 15001987]; | [evidence IEA], [evidence IDA] [pmid | a change in the relative amounts |
| | | GO: 0005578: extracellular | GO: 0008083: growth factor activity | 11427521], [evidence NAS] [pmid | of soluble VEGF-A receptor Flt-1 |
| | | matrix (sensu Metazoa) | [evidence IEA]; [evidence NAS] [pmid | 15351965]; GO: 0007399: | and VEGF-A in the circulation. |
| | | [evidence NAS] [pmid | 11016853]; GO: 0050840: extracellular | neurogenesis [evidence ISS], | Association between HER-2/neu |
| | | 14570917] | matrix binding [evidence NAS] [pmid | [evidence TAS] [pmid 15351965]; | and VEGF expression supports |
| | | | 14570917]; GO: 0042803: protein | GO: 0016477: cell migration | the use of combination therapies |
| | | | homodimerization activity [evidence NAS] | [evidence NAS] [pmid 15122338]; | directed against both HER-2/neu |
| | | | [pmid 12127077]; GO: 0005172: vascular | GO: 0008283: cell proliferation | and VEGF for treatment of breast |
| | | | endothelial growth factor receptor binding | [evidence IEA]; GO: 0001570: | cancers. |
| | | | [evidence TAS] [pmid 1711045] | vasculogenesis [evidence TAS] | |
| | | | | [pmid 15015550]; GO: 0006950: | |
| | | | | response to stress [evidence TAS] | |
| | | | | [pmid 9202027]; GO: 0007165: signal | |
| | | | | transduction [evidence TAS] [pmid | |
| | | | | 1711045]; GO: 0000074: regulation | |
| | | | | of cell cycle [evidence IEA]; | |
| | | | | GO: 0050930: induction of positive | |
| | | | | chemotaxis [evidence NAS] [pmid | |
| | | | | 12744932]; GO: 0043066: negative | |
| | | | | regulation of apoptosis [evidence | |
| | | | | IMP] [pmid 10066377], [evidence | |
| | | | | IMP] [pmid 11461089]; GO: 0008284: | |
| | | | | positive regulation of cell proliferation | |
| | | | | [evidence TAS] [pmid 9202027]; | |
| | | | | GO: 0030949: positive regulation of | |
| | | | | vascular endothelial growth factor | |
| | | | | receptor signaling pathway [evidence | |
| | | | | NAS] [pmid 10066377] | |
| NM_004363.1 | 19q13.1-q13.2 | membrane [goid 0016020] | NA | NA | white blood cells express a splice |
| | | [evidence IEA]; integral to | | | variant of CEA, which hinders |
| | | plasma membrane [goid | | | detection of tumor cell cDNA in |
| | | 0005887] [evidence TAS] | | | whole blood samples |
| | | [pmid 3814146 | | | |
| NM_019010.1 | 17q21.2 | intermediate filament [goid | structural constituent of cytoskeleton [goid | biological process unknown [goid | Alteration of CK7 and CK20 |
| | | 0005882] [evidence NAS] | 0005200] [evidence NAS] [pmid 8359595] | 0000004] [evidence ND] [pmid | expression profile that occurs |
| | | [pmid 8359595] | | 8359595] | early in small intestinal |
| | | | | | tumorigenesis. |
| NM_006636.2 | 2p13.1 | mitochondrion [goid | hydrolase activity [goid 0016787] | one-carbon compound metabolism | NA |
| | | 0005739] [evidence TAS] | [evidence IEA]; magnesium ion binding | [goid 0006730] [evidence IEA]; folic | |
| | | [pmid 8218174] | [goid 0000287] [evidence IEA]; | acid and derivative biosynthesis | |
| | | | oxidoreductase activity [goid 0016491] | [goid 0009396] [evidence IEA] | |
| | | | [evidence IEA]; electron transporter | | |
| | | | activity [goid 0005489] [evidence TAS] | | |
| | | | [pmid 8218174]; methenyltetrahydrofolate | | |
| | | | cyclohydrolase activity [goid 0004477] | | |
| | | | [evidence TAS] [pmid 8218174]; | | |
| | | | methylenetetrahydrofolate | | |
| | | | dehydrogenase (NAD+) activity [goid | | |
| | | | 0004487] [evidence IEA] | | |
| NM_003258.1 | 17q23.2-q25.3 | cytoplasm [goid 0005737] | ATP binding [goid 0005524] [evidence | DNA replication [goid ssss0006260] | Mutation analysis in the coding |
| | | [evidence NR] | IEA]; kinase activity [goid 0016301] | [evidence IEA]; nucleobase, | sequence of thymidine kinase 1 |
| | | | [evidence IEA]; nucleotide binding [goid | nucleoside, nucleotide and nucleic | in breast and colorectal cancer |
| | | | 0000166] [evidence IEA]; transferase | acid metabolism [goid 0006139] | |
| | | | activity [goid 0016740] [evidence IEA]; | [evidence TAS] [pmid 3335503] | |
| | | | thymidine kinase activity [goid 0004797] | | |
| | | | [evidence TAS] [pmid 3335503] | | |
| NM_012145.2 | 2q37.3 | NA | ATP binding [goid 0005524] [evidence | DNA metabolism [goid 0006259] | NA |
| | | | IEA]; kinase activity [goid 0016301] | [evidence NR]; cell cycle [goid | |
| | | | [evidence IEA]; nucleotide binding [goid | 0007049] [evidence TAS] [pmid | |
| | | | 0000166] [evidence IEA]; transferase | 8024690]; dTDP biosynthesis [goid | |
| | | | activity [goid 0016740] [evidence IEA]; | 0006233] [evidence IEA]; dTTP | |
| | | | thymidylate kinase activity [goid 0004798] | biosynthesis [goid 0006235] | |
| | | | [evidence TAS] [pmid 8024690] | [evidence IEA]; cell proliferation [goid | |
| | | | | 0008283] [evidence TAS] [pmid | |
| | | | | 8024690]; nucleotide biosynthesis | |
| | | | | [goid 0009165] [evidence IEA] | |
| NM_000610.3 | 11p13 | GO: 0016021: integral to | GO: 0005518: collagen binding [evidence | GO: 0007155: cell adhesion | Data demonstrate that blockade |
| | | membrane [evidence IEA]; | NAS] [PMID: 2471973]; GO: 0005540: | [evidence IEA]; GO: 0016337: cell- | of the ERK pathway suppressed |
| | | GO: 0016020: membrane | hyaluronic acid binding [evidence IEA] | cell adhesion [evidemce NAS] [PMID | the expression of matrix |
| | | [evidence IEA]; | [PMID: 1991450]; GO: 0005540: | 1922057]; GO: 0007160: cell-matrix | metalloproteinases 3, 9, and 14, |
| | | GO: 0005887: integral to | hyaluronic acid binding [evidence NAS] | adhesion [evidence NAS] [PMID | and CD44, and markedly |
| | | plasma membrane | [PMID: 1991450]; GO: 0004872: receptor | 1922057] | inhibited the invasiveness of |
| | | [evidence NAS] [PMID | activity [evidenceIEA]; GO: 000: protein | | tumor cells. |
| | | 1991450] | binding [evidenceIEA] | | |
| NM_198175.1 | 17q21.3 | nucleus [goid 0005634] | ATP binding [goid 0005524] [evidence | cell cycle [goid 0007049] [evidence | Enhanced expression of |
| | | [evidence NAS] | IEA]; ATP binding [goid 0005524] | IEA]; CTP biosynthesis [goid | nm23H(1) protein can effectively |
| | | | [evidence NAS]; DNA binding [goid | 0006241] [evidence IEA]; GTP | inhibit colon cancer metastasis |
| | | | 0003677] [evidence IC] [pmid 11555662]; | biosynthesis [goid 0006183] | and improve prognosis of |
| | | | kinase activity [goid 0016301] [evidence | [evidence IEA]; UTP biosynthesis | sporadic colon cancer patients. |
| | | | IEA]; nucleotide binding [goid 0000166] | [goid 0006228] [evidence | |
| | | | [evidence IEA]; transferase activity [goid | IEA]; nucleotide metabolism [goid | |
| | | | 0016740] [evidence IEA]; magnesium ion | 0009117] [evidence IEA]; nucleoside | |
| | | | binding [goid 0000287] [evidence IEA]; | triphosphate biosynthesis [goid | |
| | | | magnesium ion binding [goid 0000287] | 0009142] [evidence NAS] | |
| | | | [evidence IDA] [pmid 11555662]; | | |
| | | | deoxyribonuclease activity [goid 0004536] | | |
| | | | [evidence IDA] [pmid 11555662]; | | |
| | | | nucleoside diphosphate kinase activity | | |
| | | | [goid 0004550] [evidence IEA]; | | |
| | | | nucleoside diphosphate kinase activity | | |
| | | | [goid 0004550] [evidence NAS] | | |
| NM_002466.2 | 20q13.1 | nucleus [goid 0005634] | transcription factor activity [goid 0003700] | development [goid 0007275] | NA |
| | | [evidence IEA]; chromatin | [evidence TAS] [pmid 10770937] | [evidence NR]; anti-apoptosis [goid | |
| | | [goid 0000785] [evidence | | 0006916] [evidence NR]; regulation | |
| | | NR] | | of transcription, DNA-dependent | |
| | | | | [goid 0006355] [evidence IEA]; | |
| | | | | transcription from RNA polymerase II | |
| | | | | promoter [goid 0006366] [evidence | |
| | | | | NR]; regulation of progression | |
| | | | | through cell cycle [goid 0000074] | |
| | | | | [evidence NAS] [pmid 8812502] | |
| NM_001255.1 | 1p34.1 | spindle [goid 0005819] | protein binding [goid 0005515] [evidence | mitosis [goid 0007067] [evidence | Up-regulation of cdc20 is |
| | | [evidence TAS] [pmid | IPI] [pmid 14743218] | IEA]; cell division [goid 0051301] | associated with gastric cancer |
| | | 7513050] | | [evidence IEA]; ubiquitin cycle [goid | |
| | | | | 0006512] [evidence IEA]; ubiquitin- | |
| | | | | dependent protein catabolism [goid | |
| | | | | 0006511] [evidence TAS] [pmid | |
| | | | | 9682218]; regulation of progression | |
| | | | | through cell cycle [goid 0000074] | |
| | | | | [evidence TAS] [pmid 7513050] | |
| NM_004413.1 | 16q24.3 | membrane [goid 0016020] | metal ion binding [goid 0046872] | proteolysis [goid 0006508] [evidence | DPEP1 has a role in colorectal |
| | | [evidence IEA]; microsome | [evidence IEA]; metallopeptidase activity | IEA] | carcinoma |
| | | [goid 0005792] [evidence | [goid 0008237] [evidence IEA]; dipeptidyl- | | |
| | | IEA]; endoplasmic reticulum | peptidase activity [goid 0008239] | | |
| | | [goid 0005783] [evidence | [evidence IEA]; membrane dipeptidase | | |
| | | IEA] | activity [goid 0004237] [evidence TAS] | | |
| | | | [pmid 2303490] | | |
| NM_003270.2 | Xq22 | integral to membrane [goid | signal transducer activity [goid 0004871] | cell motility [goid 0006928] [evidence | |
| | | 0016021] [evidence IEA] | [evidence IMP] [pmid 12761501] | NR]; positive regulation of I-kappaB | |
| | | | | kinase/NF-kappaB cascade [goid | |
| | | | | 0043123] [evidence IMP] [pmid | |
| | | | | 12761501] | |
| NM_080820.3 | 20p11.23 | cytoplasm [goid 0005737] | hydrolase activity, acting on ester bonds | D-amino acid catabolism [goid | DUE-B, a c-myc DNA-unwinding |
| | | [evidence IEA] | [goid 0016788] [evidence IEA] | 0019478] [evidence IEA] | element-binding protein, plays an |
| | | | | | important role in replication in |
| | | | | | vivo. |
| NM_006649.2 | Xq25 | nucleus [goid 0005634] | protein binding [goid 0005515] [evidence | ribosome biogenesis [goid 0007046] | NA |
| | | [evidence IEA] | IPI] [pmid 15383276] | [evidence IEA] | |
| NM_005804.2 | 19p13.12 | nucleus [goid 0005634] | ATP binding [goid 0005524] [evidence | mRNA export from nucleus [goid | |
| | | [evidence IEA]; nucleus | IEA]; hydrolase activity [goid 0016787] | 0006406] [evidence IGI] [pmid | |
| | | [goid 0005634] [evidence | [evidence IEA]; nucleotide binding [goid | 15047853]; nuclear mRNA splicing, | |
| | | ISS] [pmid 15047853] | 0000166] [evidence IEA]; protein binding | via spliceosome [goid 0000398] | |
| | | | [goid 0005515] [evidence IPI] [pmid | [evidence IGI] [pmid 15047853] | |
| | | | 15047853]; nucleic acid binding [goid | | |
| | | | 0003676] [evidence IEA]; ATP-dependent | | |
| | | | helicase activity [goid 0008026] [evidence | | |
| | | | IEA]; ATP-dependent RNA helicase | | |
| | | | activity [goid 0004004] [evidence ISS] | | |
| | | | [pmid 15047853] | | |
| NM_003153.3 | 12q13 | nucleus [goid 0005634] | calcium ion binding [goid 0005509] | transcription [goid 0006350] | STAT6 is required for IL-4- |
| | | [evidence IEA] | [evidence IEA]; signal transducer activity | [evidence IEA]; intracellular signaling | mediated growth inhibition and |
| | | | [goid 0004871] [evidence IEA]; | cascade [goid 0007242] [evidence | induction of apoptosis in human |
| | | | transcription factor activity [goid 0003700] | IEA]; regulation of transcription from | breast cancer cells. Alterations in |
| | | | [evidence TAS] [pmid 10747856] | RNA polymerase II promoter [goid | the STAT6 pathway may play a |
| | | | | 0006357] [evidence TAS] [pmid | crucial role in the pathogenesis of |
| | | | | 8810328] | distinct subgroups of patients |
| | | | | | with Crohn's disease. |

Genes within a region know to be amplified in cancer are indicated by (Amp) next to the chromosomal location;
Genes within a region know to have loss of heterozygosity (LOH) in cancer are indicated by (LOH) next to the chromosomal location;
NA = not available

In addition, a subset of the 14 genes below may be selected for use as endogenous controls. Endogenous control candidates are selected from among those well-known in the literature as commonly constitutively expressed gene products across a wide range of tissues and biological conditions. See Kok, J B et al., Lab Invest. 2005 January; 85(1):154-9 and Janssens, N., et al., Mol. Diagn. 2004; 8(2): 107-13 which are hereby incorporated by reference in their entirety.

TABLE 4
Endogenous controls

| Genebank Accession | Abbreviated Name |
|---|---|
| NM_001101.2 | ACTB |
| NM_003194.2 | TBP |
| NM_003234.1 | TFRC |
| NM_000194.1 | HPRT1 |
| NM_004048.2 | B2M |
| NM_000190.2 | HMBS |
| NM_004168.1 | SDHA |
| NM_021009.2 | UBC |
| NM_002046.2 | GAPDH |
| NM_000181.1 | GUSB |
| NM_001002.3 | RPLPO_1 |
| NM_012423.2 | RPL13A |
| NM_003406.2 | YWHAZ |
| D38112.1 | ATPase_sub_6 |

* The ATP6 CDS is located at nucleotides [7941 . . . 8621] of D38112.1 “Homo sapiens mitochondrial DNA, complete sequence”

### Individuals and Sample Sets

Expression of gene products may be evaluated in primary tissues and/or lymph nodes; and alternatively in primary tissue and/or bone marrow samples. Additionally, expressions of gene products are evaluated in blood samples. Additionally, expressions of gene products are evaluated in fecal samples. In addition, primary tissues, lymph nodes, bone marrow, feces and blood may be used in combination.

Samples are collected retrospectively for individuals with primary or metastatic colon cancer or prospectively from individuals suspected of developing or having colon cancer or individuals at risk of having or developing colon cancer. Gene product expression profiles are evaluated on archival paraffin-preserved primary tissue from individuals who have metastatic colon cancer. As a control, primary tissues from individuals with no metastasis are evaluated.

In the studies above, both positive and negative groups of individuals have a minimum of 4-6 years follow-up information to evaluate the relation of gene product expression to disease outcome. Both groups have a representation of individuals with good outcome (no disease progression) 4-6 years after surgery, and poor outcome with disease progression (either metastatic disease or local recurrence) within 3-5 years of surgery.

Clinical information for all individuals is reported in an extensive Case Report Form (CRF) containing at least the following clinical information: Individual ID; Demographics (Age, Sex and Menopausal Status when applicable); Lymph Node status (when applicable); DNA ploidy; Clinical TNM Staging based on the modified AJCC/UICC TNM classification per CAP protocol (revision January 2004); Histopathological Type; Pathological and/or Nuclear Grade (Modified Bloom Richardson score); Pathological staging, pT size (Pathologic tumor size, size of the invasive component) based on the modified AJCC/UICC TNM classification per CAP protocol (revision January 2004); Treatment summary (date and type of surgery, chemotherapy received, radiotherapy received) and Clinical Outcome (date of evaluation, vitality at date of evaluation, disease progression status, months of disease free survival at date of evaluation and disease progression information). Additionally, the percentage of cells that are cancerous (Tum %) in the sample used for diagnosis and subsequent analysis is included.

Differential expression of gene products from Tables 2a and 2b above identifies individuals with good outcome (no disease progression) and poor outcome with disease progression (either metastatic disease or local recurrence).

### Example 1b

### Prognosis Based on Gene Product Expression in Primary Tissue

### Primary Tissue Samples

As described above, the prognosis of individuals with colon cancer is determined based on gene product expression. Primary tissues from individuals are evaluated for determining good or poor prognosis based on differential gene expression. The differential gene product expression analysis from the samples from these individuals determine good and poor outcome.

### Example 1c

### Gene Expression Analysis

### Custom Microarray Experiment—Cancer

Tissue Specific Array and Multi-Cancer Array Experiments

Custom oligonucleotide microarrays based on an 8 k chip were provided by Agilent Technologies, Inc. (Palo Alto, Calif.). The microarrays were fabricated by Agilent using their technology for the in-situ synthesis of 60mer oligonucleotides (Hughes, et al. 2001, Nature Biotechnology 19:342-347). The 60mer microarray probes were designed by Agilent, from nucleic acid sequences provided by diaDexus, using Agilent proprietary algorithms. Whenever possible two different 60mers were designed for each nucleic acid of interest.

All Tissue Specific and Multi-Cancer microarray experiments were two-color experiments and were preformed using Agilent-recommended protocols and reagents. Briefly, each microarray was hybridized with cRNAs synthesized from polyA+ RNA, isolated from cancer and normal tissues or cell lines, and labeled with fluorescent dyes Cyanine-3 (Cy3) or Cyanine-5 (Cy5) (NEN Life Science Products, Inc., Boston, Mass.) using a linear amplification method (Agilent). In each experiment the experimental sample was RNA isolated from cancer tissue from a single individual or cell line and the reference sample was a pool of RNA isolated from normal tissues of the same organ as the cancerous tissue (i.e. normal colon tissue in experiments with colon cancer or cell line samples). Hybridizations were carried out at 60° C., overnight using Agilent in-situ hybridization buffer. Following washing, arrays were scanned with a GenePix 4000B Microarray Scanner (Axon Instruments, Inc., Union City, Calif.). Each array was scanned at two PMT voltages (600 v and 550 v). The resulting images were analyzed with GenePix Pro 3.0 Microarray Acquisition and Analysis Software (Axon). Unless otherwise noted, data reported is from images generated by scanning at PMT of 600 v.

Data normalization and expression profiling were done with Expressionist software from GeneData Inc. (South San Francisco, Calif./Basel, Switzerland). Nucleic acid sequence expression analysis was performed using only experiments that met certain quality criteria. The quality criteria that experiments must meet are a combination of evaluations performed by the Expressionist software and evaluations performed manually using raw and normalized data. To evaluate raw data quality, detection limits (the mean signal for a replicated negative control+2 Standard Deviations (SD)) for each channel were calculated. The detection limit is a measure of non-specific hybridization. Acceptable detection limits were defined for each dye (<80 for Cy5 and <150 for Cy3). Arrays with poor detection limits in one or both channels were not analyzed and the experiments were repeated. T0 evaluate normalized data quality, positive control elements included in the array were utilized. These array features should have a mean ratio of 1 (no differential expression). If these features have a mean ratio of greater than 1.5-fold up or down, the experiments were not analyzed further and were repeated. In addition to traditional scatter plots demonstrating the distribution of signal in each experiment, the Expressionist software also has minimum thresholding criteria that employ user defined parameters to identify quality data. These thresholds include two distinct quality measurements: 1) minimum area percentage, which is a measure of the integrity of each spot and 2) signal to noise ratio, which ensures that the signal being measured is significantly above any background (nonspecific) signal present. Only those features that met the threshold criteria were included in the filtering and analyses carried out by Expressionist. The thresholding settings employed require a minimum area percentage of 60% [(% pixels>background+2SD)−(% pixels saturated)], and a minimum signal to noise ratio of 2.0 in both channels. Using these criteria, very low expressors, saturated features and spots with abnormally high local background were not included in analysis.

Relative expression data was collected from Expressionist based on filtering and clustering analyses. Up-regulated nucleic acid sequences were identified using criteria for the percentage of experiments in which the nucleic acid sequence is up-regulated by at least 2-fold. For cell lines, up-regulated nucleic acid sequences were identified using criteria for the percentage of experiments in which the nucleic acid sequence is up-regulated by at least 1.8-fold. In general, up-regulation in 30% of samples tested was used as a cutoff for filtering.

Two microarray experiments were preformed for each normal and cancer tissue pair. The tissue specific Array Chip for each cancer tissue is a unique microarray specific to that tissue and cancer. The Multi-Cancer Array Chip is a universal microarray that was hybridized with samples from each of the cancers (ovarian, breast, colon, lung, and prostate). See the description below for the experiments specific to the different cancers.

UniDEX1 (UD1) Chip Experiment

Custom oligonucleotide microarrays based on a 22 k chip were provided by Agilent Technologies, Inc. (Palo Alto, Calif.). The microarrays were fabricated by Agilent using their technology for the in-situ synthesis of 60mer oligonucleotides (Hughes, et al. 2001, Nature Biotechnology 19:342-347). The 60mer microarray probes were designed by Agilent, from nucleic acid sequences provided by diaDexus, using Agilent proprietary algorithms. For the UniDEX1 array, single probes were used for each nucleic acid of interest.

All UniDEX1 microarray experiments were two-color experiments and were preformed using Agilent-recommended protocols and reagents. Microarray hybridizations were performed as described above.

In each experiment the experimental sample was RNA isolated from cancer tissue or benign disease from a single individual and the reference sample was a pool of RNA isolated from normal tissues of the same organ as the cancerous or diseased tissue (i.e. normal colon tissue in experiments with colon cancer or colon diseases). Following washing, arrays were scanned as described above.

Data normalization and expression profiling were done with Expressionist software from GeneData Inc. (South San Francisco, Calif./Basel, Switzerland). Nucleic acid sequence expression analysis was performed using only experiments that met certain quality criteria. Quality assessment was performed using the Refiner module of Expressionist and the Thresholding module of the Analyst component of the Expressionist software. In addition to traditional scatter plots demonstrating the distribution of signal in each experiment, the Expressionist software also has minimum thresholding criteria that employ user defined parameters to identify quality data. These thresholds include two distinct quality measurements: 1) maximum relative error, which is a measure of the integrity of each spot and 2) signal to noise ratio, which ensures that the signal being measured is significantly above any background (nonspecific) signal present. Only those features that met the threshold criteria were included in the filtering and analyses carried out by Expressionist. The thresholding settings employed require a maximum relative error of 1, and a minimum signal to noise ratio of 2.0 in both channels. Using these criteria, very low expressors, saturated features and spots with abnormally high local background were not included in analysis.

Relative expression data was collected from Expressionist based on filtering and clustering analyses. Up-regulated and down-regulated nucleic acid sequences were identified using criteria for the percentage of experiments in which the nucleic acid sequence is up-regulated or down-regulated by at least 1.8-fold. In general, up-regulation in ˜30% of samples tested was used as a cutoff for filtering.

Each cancer or benign disease sample and the normal pool was hybridized on the UniDEX1 chip. See the description below for the experiments specific to the different cancers.

### Microarray Experiments and Data Tables

Colon Cancer Chips

For colon cancer, the Colon Array Chip and the Multi-Cancer Array Chip designs were evaluated with overlapping sets of a total of 38 samples, comparing the expression patterns of colon cancer derived polyA+ RNA to polyA+ RNA isolated from a pool of 7 normal colon tissues. For the Colon Array Chip all 38 samples (23 Ascending colon carcinomas and 15 Rectosigmoidal carcinomas including: 5 stage I cancers, 15 stage II cancers, 15 stage III and 2 stage 1V cancers, as well as 28 Grade 1/2 and 10 Grade 3 cancers) were analyzed. The histopathologic grades for cancer are classified as follows: GX, cannot be assessed; G1, well differentiated; G2, Moderately differentiated; G3, poorly differentiated; and G4, undifferentiated. AJCC Cancer Staging Handbook, 5^{th} Edition, 1998, page 9. For the Colon Array Chip analysis, samples were further divided into groups based on the expression pattern of the known colon cancer associated gene Thymidilate Synthase (TS) (13 TS up 25 TS not up). The association of TS with advanced colorectal cancer is well documented. Paradiso et al., Br J Cancer 82(3):560-7 (2000); Etienne et al., J Clin Oncol. 20(12):2832-43 (2002); Aschele et al. Clin Cancer Res. 6(12):4797-802 (2000). For the Multi-Cancer Array Chip a subset of 27 of these samples (14 Ascending colon carcinomas and 13 Rectosigmoidal carcinomas including: 3 stage I cancers, 9 stage II cancers, 13 stage III and 2 stage 1V cancers) were assessed. In addition to the tissue samples, five colon cancer cell lines (HT29, SW480, SW620, HCT-16, CaCo2) were analyzed on the Colon Array Chip.

For the colon cancer and disease experiments on the UniDEX1 (UD1) chip a total of 74 samples, comparing the expression patterns of colon cancer or disease derived RNA to RNA isolated from a pool of 9 normal colon tissues. The sample distribution was as follows: 12 early Adenomas, 9 Stage I cancers, 11 Stage II cancers, 12 Stage III cancers, 7 Metastatic cancers (6 Liver metastases and 1 metastatic lymph node), 10 Crohn's disease, 9 Ulcerative colitis (6 active, 2 inactive and 1 unspecified) and 4 adenomatous polyps (2 FAP and 2 spontaneous). The tissues were purchased from Ardais Corporation (Lexington, Mass.).

Table 5 below summarizes the results of the colon cancer microarray experiments described above. Briefly, the table is broken into two parts: over-expression and under-expression. For each section, the Genebank sequence and reporting microarray oligos are listed along with the sample groups (described above) in which at least 30% of the samples had differential expression of at least 1.8-fold. Abbreviations for sample groups are: Adenoma (AD), Stage I (St1), Stage II (St2), Stage III (St3), Metastatic (Met), Crohn's (Cr), Colitis (Col), Crohn's and Colitis (C&C).

TABLE 5

| Genebank | | | | | | | | | | Sample Groups with Down- | | | | | | | |
|---|---|---|---|---|---|---|---|---|---|---|---|---|---|---|---|---|---|
| Accession | Oligo Accession | Sample Groups with Up-Regulation | | | | | | | | Regulation | | | | | | | |
| BC021275.2 | A_23_P84596 | | | | | | | | | | St1 | St2 | St3 | | | | |
| NM_000582.2 | A_23_P7313 | | St1 | | | Met | | | | | | | | | | | |
| NM_000610.3 | A_23_P24870 | Ad | St1 | | | | | | | | | | | | | | |
| NM_001071.1 | A_23_P50096 | | St1 | | | | | | | | | | | | | | |
| NM_001255.1 | A_23_P149195 | | St1 | | | | | | | | | | | | | | |
| NM_001554.3 | A_23_P46429 | | | | | | Cro | Col | C&C | Ad | St1 | St2 | St3 | Met | | | |
| NM_001738.1 | A_23_P168916 | | | | | | Cro | Col | C&C | | | | | | | | |
| NM_002466.2 | A_23_P143184 | | St1 | St2 | St3 | | | | | | | | | | | | |
| NM_002483.3 | A_23_P218441 | Ad | St1 | St2 | St3 | Met | Cro | Col | C&C | | | | | | | | |
| NM_002483.3 | MO_14744 | Ad | St1 | St2 | St3 | Met | | Col | C&C | | | | | | | | |
| NM_002644.2 | A_23_P149517 | Ad | | | | | | | | | St1 | | | | | | |
| NM_002644.2 | MO_78971 | Ad | | | | | | | | | St1 | St2 | St3 | | | Col | |
| NM_002644.2 | MO_78972 | Ad | | | | | Cro | | | | St1 | St2 | St3 | Met | | Col | |
| NM_003153.3 | A_23_P47879 | Ad | | | St3 | | | | | | | | | | | | |
| NM_003258.1 | A_23_P107421 | Ad | St1 | St2 | St3 | | | | | | | | | | | | |
| NM_003270.2 | A_23_P171143 | | St1 | | | | | | | | | | | | | | |
| NM_004363.1 | A_23_P153301 | Ad | St1 | St2 | St3 | Met | | | | | | | | | | | |
| NM_004363.1 | MO_94127 | Ad | St1 | St2 | St3 | Met | Cro | Col | C&C | | | | | | | | |
| NM_004413.1 | A_23_P152255 | | | St2 | | | | | | Ad | St1 | | St3 | Met | | Col | |
| NM_004591.1 | A_23_P17064 | Ad | | St2 | | | | | | | | | | | | | |
| NM_004864.1 | A_23_P16523 | Ad | St1 | St2 | St3 | Met | | | | | | | | | | | |
| NM_004864.1 | MO_13539 | | St1 | St2 | | | | | | | | | | | | | |
| NM_004994.1 | A_23_P40174 | | | | | Met | Cro | | C&C | | | | | | | | |
| NM_005063.4 | MO_78600 | | St1 | St2 | St3 | | Cro | | | | | | | | | | |
| NM_005564.2 | A_23_P169437 | | St1 | St2 | | | | | | | | | | | | | |
| NM_005564.2 | MO_17852 | Ad | St1 | | St3 | | | Col | | | | | | | | | |
| NM_005727.2 | A_23_P160167 | | | | | | | Col | | | | | | | | | |
| NM_006096.2 | A_23_P20494 | | | | | | | | | Ad | St1 | St2 | St3 | | Cro | Col | C&C |
| NM_006149.2 | A_23_P254917 | | | | | | | | | | St1 | St2 | St3 | Met | Cro | | C&C |
| NM_006408.2 | A_23_P31407 | Ad | St1 | | | | Cro | Col | C&C | | | | | | | | |
| NM_006408.2 | MO_26771 | Ad | St1 | | | | Cro | | | | | | | | | | |
| NM_006408.2 | MO_33089 | | St1 | | | | Cro | | | | | | | | | | |
| NM_006408.2 | MO_41945 | | St1 | | | | Cro | | | | | | | | | | |
| NM_006418.3 | A_23_P2789 | Ad | St1 | | | | | | | | | | | | | | |
| NM_006418.3 | MO_34380 | | St1 | | | | | | | | | | | | | | |
| NM_007052.3 | A_23_P217280 | | | St2 | | | | | | | | | | | | | |
| NM_012145.2 | A_23_P123974 | | St1 | St2 | St3 | | | | | | | | | | | | |
| NM_012445.1 | A_23_P121533 | | | | | | | | | Ad | St1 | | | | | | |
| NM_017625.2 | A_23_P84388 | Ad | | | | | Cro | Col | C&C | | | | | | | | |
| NM_017625.2 | A_23_P95790 | Ad | | | | | Cro | Col | C&C | | | St2 | | | | | |
| NM_017763.3 | A_23_P3934 | Ad | St1 | St2 | St3 | | | | | | | | | | | | |
| NM_019010.1 | A_23_P66854 | | | | | | | Col | | | St1 | St2 | | | | | |
| NM_024017.3 | A_23_P27013 | Ad | | St2 | | | | | | | | | | | | | |
| NM_032044.2 | MO_35397 | Ad | | | St3 | | Cro | Col | C&C | | St1 | St2 | | | | | |
| NM_080748.1 | A_23_P143417 | | | | St3 | | | | | | | | | | Cro | | C&C |
| NM_080820.3 | A_23_P17512 | | St1 | St2 | | | | | | | | | | | | | |
| NM_138805.2 | A_23_P41145 | Ad | | | St3 | | Cro | Col | C&C | | St1 | | | | | | |
| NM_138938.1 | A_23_P119936 | | | | | | | | | Ad | St1 | St2 | St3 | Met | Cro | Col | C&C |
| NM_145306.1 | MO_103385 | | St1 | | | | | | | | | | | | | | |
| NM_198175.1 | MO_31541 | | | St2 | St3 | | | | | | | | | | | | |
| NM_198976.1 | A_23_P210649 | | | St2 | St3 | | | | | | | | | | | | |
| NM_199168.1 | A_23_P202448 | | | | | | | Col | | | | | | | | | |

For the experiments above, table 6 lists the Genebank accession, the microarray oligo ID and the location where the oligo maps to the Genebank sequence (nucleotide range and Genebank sequence length in brackets).

TABLE 6

| | | oligo position on |
|---|---|---|
| Accession | Oligo ID | sequence |
| BC021275.2 | A_23_P84596 | 463 . . . 522 [826] |
| NM_000582.2 | A_23_P7313 | 940 . . . 999 [1616] |
| NM_000610.3 | A_23_P24870 | 2461 . . . 2520 [3091] |
| NM_001071.1 | A_23_P50096 | 1326 . . . 1385 [1536] |
| NM_001255.1 | A_23_P149195 | 1590 . . . 1633 [1686] |
| NM_001554.3 | A_23_P46429 | 1582 . . . 1641 [2037] |
| NM_001738.1 | A_23_P168916 | 928 . . . 987 [1264] |
| NM_002466.2 | A_23_P143184 | 2628 . . . 2687 [2731] |
| NM_002483.3 | A_23_P218441 | 2449 . . . 2508 [2527] |
| NM_002483.3 | MO_14744 | 2270 . . . 2327 [2527] |
| NM_002644.2 | A_23_P149517 | 3011 . . . 3070 [4266] |
| NM_002644.2 | MO_78971 | 3906 . . . 3847 [4266] |
| NM_002644.2 | MO_78972 | 4080 . . . 4021 [4266] |
| NM_003153.3 | A_23_P47879 | 3460 . . . 3519 [3993] |
| NM_003258.1 | A_23_P107421 | 1350 . . . 1409 [1421] |
| NM_003270.2 | A_23_P171143 | 1522 . . . 1581 [2069] |
| NM_004363.1 | A_23_P153301 | 2028 . . . 2087 [2974] |
| NM_004363.1 | MO_94127 | 2589 . . . 2640 [2974] |
| NM_004413.1 | A_23_P152255 | 1673 . . . 1732 [1738] |
| NM_004591.1 | A_23_P17064 | 368 . . . 427 [799] |
| NM_004864.1 | A_23_P16523 | 1097 . . . 1156 [1204] |
| NM_004864.1 | MO_13539 | 1122 . . . 1175 [1204] |
| NM_004994.1 | A_23_P40174 | 2256 . . . 2315 [2334] |
| NM_005063.4 | MO_78600 | 5311 . . . 5370 [5473] |
| NM_005564.2 | A_23_P169437 | 502 . . . 561 [845] |
| NM_005564.2 | MO_17852 | 512 . . . 571 [845] |
| NM_005727.2 | A_23_P160167 | 821 . . . 880 [1297] |
| NM_006096.2 | A_23_P20494 | 2668 . . . 2727 [3074] |
| NM_006149.2 | A_23_P254917 | 688 . . . 747 [1117] |
| NM_006408.2 | A_23_P31407 | 373 . . . 432 [1701] |
| NM_006408.2 | MO_26771 | 188 . . . 247 [1701] |
| NM_006408.2 | MO_33089 | 524 . . . 583 [1701] |
| NM_006408.2 | MO_41945 | 272 . . . 331 [1701] |
| NM_006418.3 | A_23_P2789 | 1596 . . . 1655 [2844] |
| NM_006418.3 | MO_34380 | 1599 . . . 1658 [2844] |
| NM_007052.3 | A_23_P217280 | 2028 . . . 2087 [2612] |
| NM_012145.2 | A_23_P123974 | 961 . . . 1020 [1066] |
| NM_012445.1 | A_23_P121533 | 1733 . . . 1792 [1807] |
| NM_017625.2 | A_23_P84388 | 1107 . . . 1166 [1209] |
| NM_017625.2 | A_23_P95790 | 1087 . . . 1146 [1209] |
| NM_017763.3 | A_23_P3934 | 5100 . . . 5158 [5585] |
| NM_019010.1 | A_23_P66854 | 1339 . . . 1398 [1817] |
| NM_024017.3 | A_23_P27013 | 2427 . . . 2486 [2583] |
| NM_032044.2 | MO_35397 | 1228 . . . 1270 [1285] |
| NM_080748.1 | A_23_P143417 | 324 . . . 383 [602] |
| NM_080820.3 | A_23_P17512 | 1202 . . . 1261 [1344] |
| NM_138805.2 | A_23_P41145 | 1159 . . . 1218 [1322] |
| NM_138938.1 | A_23_P119936 | 768 . . . 827 [1002] |
| NM_145306.1 | MO_103385 | 984 . . . 1043 [1129] |
| NM_198175.1 | MO_31541 | 407 . . . 466 [1031] |
| NM_198976.1 | A_23_P210649 | 1994 . . . 2053 [2263] |
| NM_199168.1 | A_23_P202448 | 1496 . . . 1555 [1940] |

These results demonstrate that the gene products of the targets listed in tables 2a and 2b are differentially expressed in colon cancer and useful for the detection and prognosis colon cancer.

### Example 2

### Relative Quantitation of Gene Expression

Blood, Fecal, lymph node, fresh frozen or Formalin Fixed Paraffin Embedded (FFPE) histological samples from the individuals described above are analyzed for gene expression by QPCR methodologies known to those of skill in the art, as exemplified below.

### FFPE Samples

Specifically, one FFPE block from a primary tumor resection from each individual was selected based on maximal tumor content. A narrow tumor content range was used to minimize the effects of the presence of non-cancer cells on the expression profile. Tumor content range is expected to be between 60 to 80% of cancer cells based on the characteristics of the samples in the sample bank.

Total RNA was extracted from two whole 20 micron sections from each FFPE block or from macro-dissected material. A total of 3-4 RNA samples from colon tissue from normal individuals and 3-4 total RNA samples from normal adjacent tissues (NAT) from pathologically normal colon tissues adjacent to a tumor from an individual with colon cancer were tested to obtain a baseline level of expression for each of the gene products tested. Prior to RNA extraction, paraffin was removed from samples by a deparaffinization step consisting of a xylene extraction followed by an ethanol wash. Kits for the extraction of RNA from FFPE samples such as the Optimunm™ FFPE RNA Isolation Kit (Catalog #47000) from Ambion® Diagnostics (Austin, Tex.) are commercially available. Additionally, methodologies for processing FFPE samples are known to those of skill in the art, see Cronin et al. American Journal of Pathology, January 2004, Vol. 164, No. 1, pages 35-42. All measurements of gene products were normalized against endogenous controls.

### TaqMan™ Gene Expression Profiling

Removal of contaminating genomic DNA, quantitation of total RNA, measurements of residual genomic DNA contamination and preparation of cDNA by reverse transcription was performed prior to TaqMan™ gene expression profiling. TaqMan™ gene expression was performed on targets selected from Table 2a and 2b above.

Real-Time quantitative PCR with fluorescent Taqman® probes is a quantitation detection system utilizing the 5′-3′ nuclease activity of Taq DNA polymerase. The method uses an internal fluorescent oligonucleotide probe (Taqman®) labeled with a 5′ reporter dye and a downstream, 3′ quencher dye. During PCR, the 5′-3′ nuclease activity of Taq DNA polymerase releases the reporter, whose fluorescence can then be detected by the laser detector of a Realtime Quantitative PCR machine such as the Model 7000, 7700 or 7900 Sequence Detection System from PE Applied Biosystems (Foster City, Calif., USA). Amplification of an endogenous control(s) is used to standardize the amount of sample RNA added to the reaction and normalize for Reverse Transcriptase (RT) efficiency. Gene products from Table 4 above were used as endogenous control(s).

To calculate relative quantitation between all the samples studied, the target RNA levels for one sample can be used as the basis for comparative results (calibrator). Quantitation relative to the “calibrator” can be obtained using the comparative method (User Bulletin #2: ABI PRISM 7700 Sequence Detection System).

The tissue distribution and the level of the target gene are evaluated for every sample in normal and cancer tissues. Total RNA is extracted from normal tissues, cancer tissues, and from cancers and the corresponding matched adjacent tissues. Subsequently, first strand cDNA is prepared with reverse transcriptase and the polymerase chain reaction is done using primers and Taqman® probes specific to each target gene. The results are analyzed using the ABI PRISM 7700 Sequence Detector. The absolute numbers are relative levels of expression of the target gene in a particular tissue compared to the calibrator tissue.

One of ordinary skill can design appropriate primers using commercially available software such as Primer Express® 2.0 from Applied Biosystems (Foster City, Calif.) or Oligo® version 5 or 6 from Molecular Biology Insights, Inc (Cascade, Colo.). Criteria for designing primers are known to those of skill in the art, see Cronin et al. American Journal of Pathology, January 2004, Vol. 164, No. 1, pages 35-42.

The relative levels of expression of the gene in normal tissues versus other cancer tissues can then be determined. All the values are compared to the calibrator. Normal RNA samples are commercially available pools, originated by pooling samples of a particular tissue from different individuals. The expression of each gene was normalized against one or more endogenous controls as described above.

Alternatively, to compare expression profiles between specimens, normalization based on endogenous controls is used to correct for differences arising from variability in RNA quality and total quantity of RNA in each assay. A reference CT (threshold cycle) for each tested specimen is defined as the average measured CT of the endogenous controls. In an approach similar to what has been described by others, endogenous controls are selected for use from among several candidate reference genes tested in this assay. See Vandesompele J, et al., Genome Biol 2002, 3: RESEARCH0034. The endogenous controls selected for the final analysis show the lowest levels of expression variability among the individual specimens tested. An average of multiple gene products is used to minimize the risk of normalization bias that can result from variation in expression of any single reference gene. See Suzuki T, et al., Biotechniques 29:332-337 (2000). Relative mRNA level of a test gene within a tissue specimen is defined as 2^{ΔCT}+10.0, where ΔCT=CT (test gene)−CT (mean of endogenous controls). Unless indicated otherwise, normalized expression is represented on a scale in which the average expression of the endogenous controls is 10, corresponding to a mean CT of 30.7.

Table 7 below lists the components of each QPCR experiment performed on the genes described above. In some cases, multiple experiments have been designed for a single gene. The table includes the GeneBank Accession for each gene, the SEQ ID NO and DDXS Accession for the amplified and detected portion of the gene, the DDXS nomenclature for the amplicon, the SEQ ID NO and DDXS Accession for the QPCR forward primer, the SEQ ID NO and DDXS Accession for the QPCR reverse primer and SEQ ID NO and DDXS Accession for the QPCR probe. Experiments are grouped by accession. For example, in a QPCR experiment for GeneBank accession NM_##### the amplified and detected sequence is annotated as accession DEX0593_XXX.nt. 1, the forward primer is DEX0593_XXX.nt.2, the reverse primer is DEX0593_XXX.nt.3 and the probe is DEX0593_XXX.nt.4.

TABLE 7

| | SEQ | | | SEQ | |
|---|---|---|---|---|---|
| Genebank | ID | DDXS Amplicon | | ID | DDXS Forward |
| Accession | NO | Accession | DDXS Amplicon | NO | Primer Accession |
| NM_032044.2 | 1 | DEX0593_001.nt.1 | Cln101.amp.1 | 2 | DEX0593_001.nt.2 |
| NM_007052.3 | 5 | DEX0593_002.nt.1 | Cln106.amp.1 | 6 | DEX0593_002.nt.2 |
| NM_004363.1 | 9 | DEX0593_003.nt.1 | Cln224v1.amp.1 | 10 | DEX0593_003.nt.2 |
| NM_033229.1 | 13 | DEX0593_004.nt.1 | Cln129.amp.1 | 14 | DEX0593_004.nt.2 |
| AC023992.8 | 17 | DEX0593_005.nt.1 | Cln242v1.amp.1 | 18 | DEX0593_005.nt.2 |
| AL359752.11 | 21 | DEX0593_006.nt.1 | Cln101V1.amp.1 | 22 | DEX0593_006.nt.2 |
| NM_080748.1 | 25 | DEX0593_007.nt.1 | Cln254.amp.1 | 26 | DEX0593_007.nt.2 |
| NM_080748.1 | 29 | DEX0593_008.nt.1 | Cln254a.amp.1 | 30 | DEX0593_008.nt.2 |
| NM_138805.2 | 33 | DEX0593_009.nt.1 | Cln108.amp.1 | 34 | DEX0593_009.nt.2 |
| NM_138805.2 | 37 | DEX0593_010.nt.1 | Cln108b.amp.1 | 38 | DEX0593_010.nt.2 |
| NM_138805.2 | 41 | DEX0593_011.nt.1 | Cln108c.amp.1 | 42 | DEX0593_011.nt.2 |
| NM_006418.3 | 45 | DEX0593_012.nt.1 | Cln109c.amp.1 | 46 | DEX0593_012.nt.2 |
| NM_006418.3 | 49 | DEX0593_013.nt.1 | Cln109.amp.1 | 50 | DEX0593_013.nt.2 |
| NM_006418.3 | 53 | DEX0593_014.nt.1 | Cln109B.amp.1 | 54 | DEX0593_014.nt.2 |
| NM_024017.3 | 57 | DEX0593_015.nt.1 | Cln130.amp.1 | 58 | DEX0593_015.nt.2 |
| NM_024017.3 | 61 | DEX0593_016.nt.1 | Cln130a.amp.1 | 62 | DEX0593_016.nt.2 |
| NM_006149.2 | 65 | DEX0593_017.nt.1 | Cln114.amp.1 | 66 | DEX0593_017.nt.2 |
| NM_001738.1; | 69 | DEX0593_018.nt.1 | Cln115.amp.1 | 70 | DEX0593_018.nt.2 |
| M33987.1 | | | | | |
| AY358469.1 | 73 | DEX0593_019.nt.1 | Cln124.amp.1 | 74 | DEX0593_019.nt.2 |
| NM_017716.1 | 77 | DEX0593_020.nt.1 | Cln125.amp.1 | 78 | DEX0593_020.nt.2 |
| NM_002644.2 | 81 | DEX0593_021.nt.1 | Cln113.amp.1 | 82 | DEX0593_021.nt.2 |
| NM_017625.2 | 85 | DEX0593_022.nt.1 | DSH505.amp.1 | 86 | DEX0593_022.nt.2 |
| NM_031457.1 | 89 | DEX0593_023.nt.1 | DSH510.amp.1 | 90 | DEX0593_023.nt.2 |
| NM_005727.2 | 93 | DEX0593_024.nt.1 | DSH522.amp.1 | 94 | DEX0593_024.nt.2 |
| NM_003823.2 | 97 | DEX0593_025.nt.1 | Cln248.amp.1 | 98 | DEX0593_025.nt.2 |
| NM_001415.2 | 101 | DEX0593_026.nt.1 | Cln243.amp.1 | 102 | DEX0593_026.nt.2 |
| NM_012155.1 | 105 | DEX0593_027.nt.1 | Cln264.amp.1 | 106 | DEX0593_027.nt.2 |
| NM_000582.2 | 109 | DEX0593_028.nt.1 | Cln245.amp.1 | 110 | DEX0593_028.nt.2 |
| NM_032023.3 | 113 | DEX0593_029.nt.1 | Ovr216.amp.1 | 114 | DEX0593_029.nt.2 |
| NM_144947.1 | 117 | DEX0593_030.nt.1 | DSH38.amp.1 | 118 | DEX0593_030.nt.2 |
| AC084847.5 | 121 | DEX0593_031.nt.1 | Cln237v1.amp.1 | 122 | DEX0593_031.nt.2 |
| NM_017763.3; | 125 | DEX0593_032.nt.1 | Cln242.amp.1 | 126 | DEX0593_032.nt.2 |
| AB081837.1 | | | | | |
| AJ236922.1 | 129 | DEX0593_033.nt.1 | Cln260.amp.1 | 130 | DEX0593_033.nt.2 |
| NM_002483.3 | 133 | DEX0593_034.nt.1 | Cln263.amp.1 | 134 | DEX0593_034.nt.2 |
| NM_006408.2 | 137 | DEX0593_035.nt.1 | Mam111.amp.1 | 138 | DEX0593_035.nt.2 |
| NM_004864.1 | 141 | DEX0593_036.nt.1 | Pcan065.amp.1 | 142 | DEX0593_036.nt.2 |
| NM_012445.1 | 145 | DEX0593_037.nt.1 | Pro108a.amp.1 | 146 | DEX0593_037.nt.2 |
| NM_138938.1 | 149 | DEX0593_038.nt.1 | Pcan041.amp.1 | 150 | DEX0593_038.nt.2 |
| BC070213.1 | 153 | DEX0593_039.nt.1 | Pcan047b.amp.1 | 154 | DEX0593_039.nt.2 |
| NM_006475.1 | 157 | DEX0593_040.nt.1 | Cln252.amp.1 | 158 | DEX0593_040.nt.2 |
| NM_004385.2 | 161 | DEX0593_041.nt.1 | Pcan045.amp.1 | 162 | DEX0593_041.nt.2 |
| NM_004385.2 | 165 | DEX0593_042.nt.1 | Pcan045b.amp.1 | 166 | DEX0593_042.nt.2 |
| BC021275.2 | 169 | DEX0593_043.nt.1 | Pcan039b.amp.1 | 170 | DEX0593_043.nt.2 |
| NM_005408.2 | 173 | DEX0593_044.nt.1 | DSH82/83.amp.1 | 174 | DEX0593_044.nt.2 |
| NM_018098.4 | 177 | DEX0593_045.nt.1 | Cln176b.amp.1 | 178 | DEX0593_045.nt.2 |
| NM_006645.1 | 181 | DEX0593_046.nt.1 | DEX0451_037.nt.3.amp.1 | 182 | DEX0593_046.nt.2 |
| NM_004625.3 | 185 | DEX0593_047.nt.1 | Ovr212a.amp.1 | 186 | DEX0593_047.nt.2 |
| NM_001008540.1 | 189 | DEX0593_048.nt.1 | DSH862.amp.1 | 190 | DEX0593_048.nt.2 |
| NM_000579.1 | 193 | DEX0593_049.nt.1 | DSH51.amp.1 | 194 | DEX0593_049.nt.2 |
| NM_004367.3 | 197 | DEX0593_050.nt.1 | DSH106.amp.1 | 198 | DEX0593_050.nt.2 |
| NM_004591.1 | 201 | DEX0593_051.nt.1 | DSH73.amp.1 | 202 | DEX0593_051.nt.2 |
| NM_006564.1 | 205 | DEX0593_052.nt.1 | DSH105.amp.1 | 206 | DEX0593_052.nt.2 |
| NM_178445.1 | 209 | DEX0593_053.nt.1 | DSH97.amp.1 | 210 | DEX0593_053.nt.2 |
| NM_003965.3 | 213 | DEX0593_054.nt.1 | DSH209.amp.1 | 214 | DEX0593_054.nt.2 |
| NM_001838.2 | 217 | DEX0593_055.nt.1 | DSH859.amp.1 | 218 | DEX0593_055.nt.2 |
| NM_002989.2 | 221 | DEX0593_056.nt.1 | DSH89.amp.1 | 222 | DEX0593_056.nt.2 |
| NM_001554.3 | 225 | DEX0593_057.nt.1 | Ovr235c.amp.1 | 226 | DEX0593_057.nt.2 |
| AY327584.1 | 229 | DEX0593_058.nt.1 | Mam096.amp.1 | 230 | DEX0593_058.nt.2 |
| NM_006988.3 | 233 | DEX0593_059.nt.1 | DSH607.amp.1 | 234 | DEX0593_059.nt.2 |
| NM_001571.2 | 237 | DEX0593_060.nt.1 | DSH371.amp.1 | 238 | DEX0593_060.nt.2 |
| NM_145306.1 | 241 | DEX0593_061.nt.1 | Pcan035.amp.1 | 242 | DEX0593_061.nt.2 |
| BC042754.1 | 245 | DEX0593_062.nt.1 | DSH196.amp.1 | 246 | DEX0593_062.nt.2 |
| NM_001908.3 | 249 | DEX0593_063.nt.1 | DSH223/CTSB.amp.1 | 250 | DEX0593_063.nt.2 |
| NM_031419.2 | 253 | DEX0593_064.nt.1 | DSH198.amp.1 | 254 | DEX0593_064.nt.2 |
| NM_006096.2 | 257 | DEX0593_065.nt.1 | DSH207.amp.1 | 258 | DEX0593_065.nt.2 |
| NM_006096.2 | 261 | DEX0593_066.nt.1 | DSH207a.amp.1 | 262 | DEX0593_066.nt.2 |
| NM_207520.1 | 265 | DEX0593_067.nt.1 | DSH211.amp.1 | 266 | DEX0593_067.nt.2 |
| NM_005063.4 | 269 | DEX0593_068.nt.1 | DSH226.amp.1 | 270 | DEX0593_068.nt.2 |
| NM_198976.1 | 273 | DEX0593_069.nt.1 | DSH248.amp.1 | 274 | DEX0593_069.nt.2 |
| CR749471.1 | 277 | DEX0593_070.nt.1 | DSH250.amp.1 | 278 | DEX0593_070.nt.2 |
| CR749471.1 | 281 | DEX0593_071.nt.1 | DSH250a.amp.1 | 282 | DEX0593_071.nt.2 |
| AC021236.10 | 285 | DEX0593_072.nt.1 | DSH260.amp.1 | 286 | DEX0593_072.nt.2 |
| NM_024918.2 | 289 | DEX0593_073.nt.1 | DSH279.amp.1 | 290 | DEX0593_073.nt.2 |
| AC093619.5 | 293 | DEX0593_074.nt.1 | DSH282.amp.1 | 294 | DEX0593_074.nt.2 |
| NM_005564.2 | 297 | DEX0593_075.nt.1 | DSH330.amp.1 | 298 | DEX0593_075.nt.2 |
| AY623117.1 | 301 | DEX0593_076.nt.1 | DSH811a.amp.1 | 302 | DEX0593_076.nt.2 |
| NM_005201.2 | 305 | DEX0593_077.nt.1 | DSH375.amp.1 | 306 | DEX0593_077.nt.2 |
| NM_139276.2 | 309 | DEX0593_078.nt.1 | DSH265.amp.1 | 310 | DEX0593_078.nt.2 |
| NM_004994.1 | 313 | DEX0593_079.nt.1 | MMP9.amp.1 | 314 | DEX0593_079.nt.2 |
| NM_003219.1 | 317 | DEX0593_080.nt.1 | TERT.amp.1 | 318 | DEX0593_080.nt.2 |
| NM_001071.1 | 321 | DEX0593_081.nt.1 | TS.amp.1 | 322 | DEX0593_081.nt.2 |
| NM_198496.1 | 325 | DEX0593_082.nt.1 | AMACO.amp.1 | 326 | DEX0593_082.nt.2 |
| NM_199168.1 | 329 | DEX0593_083.nt.1 | CXCL12.amp.1 | 330 | DEX0593_083.nt.2 |
| NM_022059.1 | 333 | DEX0593_084.nt.1 | CXCL16.amp.1 | 334 | DEX0593_084.nt.2 |
| NM_003376.3 | 337 | DEX0593_085.nt.1 | VEGF.amp.1 | 338 | DEX0593_085.nt.2 |
| NM_004363.1 | 341 | DEX0593_086.nt.1 | CEACAM5.amp.1 | 342 | DEX0593_086.nt.2 |
| NM_019010.1 | 345 | DEX0593_087.nt.1 | KRT20.amp.1 | 346 | DEX0593_087.nt.2 |
| NM_006636.2 | 349 | DEX0593_088.nt.1 | MTHFD2.amp.1 | 350 | DEX0593_088.nt.2 |
| NM_003258.1 | 353 | DEX0593_089.nt.1 | TK1.amp.1 | 354 | DEX0593_089.nt.2 |
| NM_012145.2 | 357 | DEX0593_090.nt.1 | DTYMK.amp.1 | 358 | DEX0593_090.nt.2 |
| NM_000610.3 | 361 | DEX0593_091.nt.1 | CD44.amp.1 | 362 | DEX0593_091.nt.2 |
| NM_198175.1 | 365 | DEX0593_092.nt.1 | NME1.amp.1 | 366 | DEX0593_092.nt.2 |
| NM_002466.2 | 369 | DEX0593_093.nt.1 | MYBL2.amp.1 | 370 | DEX0593_093.nt.2 |
| NM_001255.1 | 373 | DEX0593_094.nt.1 | CDC20.amp.1 | 374 | DEX0593_094.nt.2 |
| NM_004413.1 | 377 | DEX0593_095.nt.1 | DPEP1.amp.1 | 378 | DEX0593_095.nt.2 |
| NM_003270.2 | 381 | DEX0593_096.nt.1 | TSPAN6.amp.1 | 382 | DEX0593_096.nt.2 |
| NM_080820.3 | 385 | DEX0593_097.nt.1 | HARS2.amp.1 | 386 | DEX0593_097.nt.2 |
| NM_006649.2 | 389 | DEX0593_098.nt.1 | UTP14A.amp.1 | 390 | DEX0593_098.nt.2 |
| NM_005804.2 | 393 | DEX0593_099.nt.1 | DDX39.amp.1 | 394 | DEX0593_099.nt.2 |
| NM_003153.3 | 397 | DEX0593_100.nt.1 | STAT6.amp.1 | 398 | DEX0593_100.nt.2 |
| NM_001101.2 | 401 | DEX0593_101.nt.1 | ACTB.amp.1 | 402 | DEX0593_101.nt.2 |
| NM_003194.2 | 405 | DEX0593_102.nt.1 | TBP.amp.1 | 406 | DEX0593_102.nt.2 |
| NM_003234.1 | 409 | DEX0593_103.nt.1 | TFRC.amp.1 | 410 | DEX0593_103.nt.2 |
| NM_000194.1 | 413 | DEX0593_104.nt.1 | HPRT1.amp.1 | 414 | DEX0593_104.nt.2 |
| NM_004048.2 | 417 | DEX0593_105.nt.1 | B2M.amp.1 | 418 | DEX0593_105.nt.2 |
| NM_000190.2 | 421 | DEX0593_106.nt.1 | HMBS.amp.1 | 422 | DEX0593_106.nt.2 |
| NM_000190.2 | 425 | DEX0593_107.nt.1 | HMBS2.amp.1 | 426 | DEX0593_107.nt.2 |
| NM_004168.1 | 429 | DEX0593_108.nt.1 | SDHA.amp.1 | 430 | DEX0593_108.nt.2 |
| NM_004168.1 | 433 | DEX0593_109.nt.1 | SDHA2.amp.1 | 434 | DEX0593_109.nt.2 |
| NM_021009.2 | 437 | DEX0593_110.nt.1 | UBC.amp.1 | 438 | DEX0593_110.nt.2 |
| NM_002046.2 | 441 | DEX0593_111.nt.1 | GAPDH.amp.1 | 442 | DEX0593_111.nt.2 |
| NM_000181.1 | 445 | DEX0593_112.nt.1 | GUSB.amp.1 | 446 | DEX0593_112.nt.2 |
| NM_001002.3 | 449 | DEX0593_113.nt.1 | RPLPO_1.amp.1 | 450 | DEX0593_113.nt.2 |
| NM_012423.2 | 453 | DEX0593_114.nt.1 | RPL13A.amp.1 | 454 | DEX0593_114.nt.2 |
| NM_003406.2 | 457 | DEX0593_115.nt.1 | YWHAZ.amp.1 | 458 | DEX0593_115.nt.2 |
| D38112.1 | 461 | DEX0593_116.nt.1 | ATPase_sub_6.amp.1 | 462 | DEX0593_116.nt.2 |

| | SEQ | | SEQ | |
|---|---|---|---|---|
| Genebank | ID | DDXS Reverse | ID | DDXS Probe |
| Accession | NO | Primer Accession | NO | Accession |
| NM_032044.2 | 3 | DEX0593_001.nt.3 | 4 | DEX0593_001.nt.4 |
| NM_007052.3 | 7 | DEX0593_002.nt.3 | 8 | DEX0593_002.nt.4 |
| NM_004363.1 | 11 | DEX0593_003.nt.3 | 12 | DEX0593_003.nt.4 |
| NM_033229.1 | 15 | DEX0593_004.nt.3 | 16 | DEX0593_004.nt.4 |
| AC023992.8 | 19 | DEX0593_005.nt.3 | 20 | DEX0593_005.nt.4 |
| AL359752.11 | 23 | DEX0593_006.nt.3 | 24 | DEX0593_006.nt.4 |
| NM_080748.1 | 27 | DEX0593_007.nt.3 | 28 | DEX0593_007.nt.4 |
| NM_080748.1 | 31 | DEX0593_008.nt.3 | 32 | DEX0593_008.nt.4 |
| NM_138805.2 | 35 | DEX0593_009.nt.3 | 36 | DEX0593_009.nt.4 |
| NM_138805.2 | 39 | DEX0593_010.nt.3 | 40 | DEX0593_010.nt.4 |
| NM_138805.2 | 43 | DEX0593_011.nt.3 | 44 | DEX0593_011.nt.4 |
| NM_006418.3 | 47 | DEX0593_012.nt.3 | 48 | DEX0593_012.nt.4 |
| NM_006418.3 | 51 | DEX0593_013.nt.3 | 52 | DEX0593_013.nt.4 |
| NM_006418.3 | 55 | DEX0593_014.nt.3 | 56 | DEX0593_014.nt.4 |
| NM_024017.3 | 59 | DEX0593_015.nt.3 | 60 | DEX0593_015.nt.4 |
| NM_024017.3 | 63 | DEX0593_016.nt.3 | 64 | DEX0593_016.nt.4 |
| NM_006149.2 | 67 | DEX0593_017.nt.3 | 68 | DEX0593_017.nt.4 |
| NM_001738.1; | 71 | DEX0593_018.nt.3 | 72 | DEX0593_018.nt.4 |
| M33987.1 | | | | |
| AY358469.1 | 75 | DEX0593_019.nt.3 | 76 | DEX0593_019.nt.4 |
| NM_017716.1 | 79 | DEX0593_020.nt.3 | 80 | DEX0593_020.nt.4 |
| NM_002644.2 | 83 | DEX0593_021.nt.3 | 84 | DEX0593_021.nt.4 |
| NM_017625.2 | 87 | DEX0593_022.nt.3 | 88 | DEX0593_022.nt.4 |
| NM_031457.1 | 91 | DEX0593_023.nt.3 | 92 | DEX0593_023.nt.4 |
| NM_005727.2 | 95 | DEX0593_024.nt.3 | 96 | DEX0593_024.nt.4 |
| NM_003823.2 | 99 | DEX0593_025.nt.3 | 100 | DEX0593_025.nt.4 |
| NM_001415.2 | 103 | DEX0593_026.nt.3 | 104 | DEX0593_026.nt.4 |
| NM_012155.1 | 107 | DEX0593_027.nt.3 | 108 | DEX0593_027.nt.4 |
| NM_000582.2 | 111 | DEX0593_028.nt.3 | 112 | DEX0593_028.nt.4 |
| NM_032023.3 | 115 | DEX0593_029.nt.3 | 116 | DEX0593_029.nt.4 |
| NM_144947.1 | 119 | DEX0593_030.nt.3 | 120 | DEX0593_030.nt.4 |
| AC084847.5 | 123 | DEX0593_031.nt.3 | 124 | DEX0593_031.nt.4 |
| NM_017763.3; | 127 | DEX0593_032.nt.3 | 128 | DEX0593_032.nt.4 |
| AB081837.1 | | | | |
| AJ236922.1 | 131 | DEX0593_033.nt.3 | 132 | DEX0593_033.nt.4 |
| NM_002483.3 | 135 | DEX0593_034.nt.3 | 136 | DEX0593_034.nt.4 |
| NM_006408.2 | 139 | DEX0593_035.nt.3 | 140 | DEX0593_035.nt.4 |
| NM_004864.1 | 143 | DEX0593_036.nt.3 | 144 | DEX0593_036.nt.4 |
| NM_012445.1 | 147 | DEX0593_037.nt.3 | 148 | DEX0593_037.nt.4 |
| NM_138938.1 | 151 | DEX0593_038.nt.3 | 152 | DEX0593_038.nt.4 |
| BC070213.1 | 155 | DEX0593_039.nt.3 | 156 | DEX0593_039.nt.4 |
| NM_006475.1 | 159 | DEX0593_040.nt.3 | 160 | DEX0593_040.nt.4 |
| NM_004385.2 | 163 | DEX0593_041.nt.3 | 164 | DEX0593_041.nt.4 |
| NM_004385.2 | 167 | DEX0593_042.nt.3 | 168 | DEX0593_042.nt.4 |
| BC021275.2 | 171 | DEX0593_043.nt.3 | 172 | DEX0593_043.nt.4 |
| NM_005408.2 | 175 | DEX0593_044.nt.3 | 176 | DEX0593_044.nt.4 |
| NM_018098.4 | 179 | DEX0593_045.nt.3 | 180 | DEX0593_045.nt.4 |
| NM_006645.1 | 183 | DEX0593_046.nt.3 | 184 | DEX0593_046.nt.4 |
| NM_004625.3 | 187 | DEX0593_047.nt.3 | 188 | DEX0593_047.nt.4 |
| NM_001008540.1 | 191 | DEX0593_048.nt.3 | 192 | DEX0593_048.nt.4 |
| NM_000579.1 | 195 | DEX0593_049.nt.3 | 196 | DEX0593_049.nt.4 |
| NM_004367.3 | 199 | DEX0593_050.nt.3 | 200 | DEX0593_050.nt.4 |
| NM_004591.1 | 203 | DEX0593_051.nt.3 | 204 | DEX0593_051.nt.4 |
| NM_006564.1 | 207 | DEX0593_052.nt.3 | 208 | DEX0593_052.nt.4 |
| NM_178445.1 | 211 | DEX0593_053.nt.3 | 212 | DEX0593_053.nt.4 |
| NM_003965.3 | 215 | DEX0593_054.nt.3 | 216 | DEX0593_054.nt.4 |
| NM_001838.2 | 219 | DEX0593_055.nt.3 | 220 | DEX0593_055.nt.4 |
| NM_002989.2 | 223 | DEX0593_056.nt.3 | 224 | DEX0593_056.nt.4 |
| NM_001554.3 | 227 | DEX0593_057.nt.3 | 228 | DEX0593_057.nt.4 |
| AY327584.1 | 231 | DEX0593_058.nt.3 | 232 | DEX0593_058.nt.4 |
| NM_006988.3 | 235 | DEX0593_059.nt.3 | 236 | DEX0593_059.nt.4 |
| NM_001571.2 | 239 | DEX0593_060.nt.3 | 240 | DEX0593_060.nt.4 |
| NM_145306.1 | 243 | DEX0593_061.nt.3 | 244 | DEX0593_061.nt.4 |
| BC042754.1 | 247 | DEX0593_062.nt.3 | 248 | DEX0593_062.nt.4 |
| NM_001908.3 | 251 | DEX0593_063.nt.3 | 252 | DEX0593_063.nt.4 |
| NM_031419.2 | 255 | DEX0593_064.nt.3 | 256 | DEX0593_064.nt.4 |
| NM_006096.2 | 259 | DEX0593_065.nt.3 | 260 | DEX0593_065.nt.4 |
| NM_006096.2 | 263 | DEX0593_066.nt.3 | 264 | DEX0593_066.nt.4 |
| NM_207520.1 | 267 | DEX0593_067.nt.3 | 268 | DEX0593_067.nt.4 |
| NM_005063.4 | 271 | DEX0593_068.nt.3 | 272 | DEX0593_068.nt.4 |
| NM_198976.1 | 275 | DEX0593_069.nt.3 | 276 | DEX0593_069.nt.4 |
| CR749471.1 | 279 | DEX0593_070.nt.3 | 280 | DEX0593_070.nt.4 |
| CR749471.1 | 283 | DEX0593_071.nt.3 | 284 | DEX0593_071.nt.4 |
| AC021236.10 | 287 | DEX0593_072.nt.3 | 288 | DEX0593_072.nt.4 |
| NM_024918.2 | 291 | DEX0593_073.nt.3 | 292 | DEX0593_073.nt.4 |
| AC093619.5 | 295 | DEX0593_074.nt.3 | 296 | DEX0593_074.nt.4 |
| NM_005564.2 | 299 | DEX0593_075.nt.3 | 300 | DEX0593_075.nt.4 |
| AY623117.1 | 303 | DEX0593_076.nt.3 | 304 | DEX0593_076.nt.4 |
| NM_005201.2 | 307 | DEX0593_077.nt.3 | 308 | DEX0593_077.nt.4 |
| NM_139276.2 | 311 | DEX0593_078.nt.3 | 312 | DEX0593_078.nt.4 |
| NM_004994.1 | 315 | DEX0593_079.nt.3 | 316 | DEX0593_079.nt.4 |
| NM_003219.1 | 319 | DEX0593_080.nt.3 | 320 | DEX0593_080.nt.4 |
| NM_001071.1 | 323 | DEX0593_081.nt.3 | 324 | DEX0593_081.nt.4 |
| NM_198496.1 | 327 | DEX0593_082.nt.3 | 328 | DEX0593_082.nt.4 |
| NM_199168.1 | 331 | DEX0593_083.nt.3 | 332 | DEX0593_083.nt.4 |
| NM_022059.1 | 335 | DEX0593_084.nt.3 | 336 | DEX0593_084.nt.4 |
| NM_003376.3 | 339 | DEX0593_085.nt.3 | 340 | DEX0593_085.nt.4 |
| NM_004363.1 | 343 | DEX0593_086.nt.3 | 344 | DEX0593_086.nt.4 |
| NM_019010.1 | 347 | DEX0593_087.nt.3 | 348 | DEX0593_087.nt.4 |
| NM_006636.2 | 351 | DEX0593_088.nt.3 | 352 | DEX0593_088.nt.4 |
| NM_003258.1 | 355 | DEX0593_089.nt.3 | 356 | DEX0593_089.nt.4 |
| NM_012145.2 | 359 | DEX0593_090.nt.3 | 360 | DEX0593_090.nt.4 |
| NM_000610.3 | 363 | DEX0593_091.nt.3 | 364 | DEX0593_091.nt.4 |
| NM_198175.1 | 367 | DEX0593_092.nt.3 | 368 | DEX0593_092.nt.4 |
| NM_002466.2 | 371 | DEX0593_093.nt.3 | 372 | DEX0593_093.nt.4 |
| NM_001255.1 | 375 | DEX0593_094.nt.3 | 376 | DEX0593_094.nt.4 |
| NM_004413.1 | 379 | DEX0593_095.nt.3 | 380 | DEX0593_095.nt.4 |
| NM_003270.2 | 383 | DEX0593_096.nt.3 | 384 | DEX0593_096.nt.4 |
| NM_080820.3 | 387 | DEX0593_097.nt.3 | 388 | DEX0593_097.nt.4 |
| NM_006649.2 | 391 | DEX0593_098.nt.3 | 392 | DEX0593_098.nt.4 |
| NM_005804.2 | 395 | DEX0593_099.nt.3 | 396 | DEX0593_099.nt.4 |
| NM_003153.3 | 399 | DEX0593_100.nt.3 | 400 | DEX0593_100.nt.4 |
| NM_001101.2 | 403 | DEX0593_101.nt.3 | 404 | DEX0593_101.nt.4 |
| NM_003194.2 | 407 | DEX0593_102.nt.3 | 408 | DEX0593_102.nt.4 |
| NM_003234.1 | 411 | DEX0593_103.nt.3 | 412 | DEX0593_103.nt.4 |
| NM_000194.1 | 415 | DEX0593_104.nt.3 | 416 | DEX0593_104.nt.4 |
| NM_004048.2 | 419 | DEX0593_105.nt.3 | 420 | DEX0593_105.nt.4 |
| NM_000190.2 | 423 | DEX0593_106.nt.3 | 424 | DEX0593_106.nt.4 |
| NM_000190.2 | 427 | DEX0593_107.nt.3 | 428 | DEX0593_107.nt.4 |
| NM_004168.1 | 431 | DEX0593_108.nt.3 | 432 | DEX0593_108.nt.4 |
| NM_004168.1 | 435 | DEX0593_109.nt.3 | 436 | DEX0593_109.nt.4 |
| NM_021009.2 | 439 | DEX0593_110.nt.3 | 440 | DEX0593_110.nt.4 |
| NM_002046.2 | 443 | DEX0593_111.nt.3 | 444 | DEX0593_111.nt.4 |
| NM_000181.1 | 447 | DEX0593_112.nt.3 | 448 | DEX0593_112.nt.4 |
| NM_001002.3 | 451 | DEX0593_113.nt.3 | 452 | DEX0593_113.nt.4 |
| NM_012423.2 | 455 | DEX0593_114.nt.3 | 456 | DEX0593_114.nt.4 |
| NM_003406.2 | 459 | DEX0593_115.nt.3 | 460 | DEX0593_115.nt.4 |
| D38112.1 | 463 | DEX0593_116.nt.3 | 464 | DEX0593_116.nt.4 |

### Expression Results

Expression results for several gene products measured by QPCR in samples from individuals are determined. Data is presented as relative expression using a Human Reference sample as a calibrator, which is assigned a value of one (1) for all other samples to be calibrated against. All expression data is normalized using the geometric mean of 2 endogenous controls in Table 4.

Over-expression levels of gene products selected from Table 2a and 2b above of a particular threshold are indicative of poor outcome and recurrence of disease within 5 years of surgery. More particularly, gene products selected from Table 2a or 2b under a particular expression threshold are indicative of poor outcome and recurrence of disease within 5 years of surgery. Statistical analysis is based on a student t-test. Additionally, the results indicate that combinations of two or more of the gene products listed in Table 2a and 2b can be used to determine likelihood of long-term survival and therapy response for an individual.

Normalized gene product expression values from the experiments described above are used to study the existence of correlation of each individual gene product with overall outcome. Gene products identified as relevant for the prediction of outcome are evaluated in a multivariate model as predictors of prognosis. Analyses conducted include: Principal Component Analysis, classification algorithms; calculation of survival rates at 5 years by prognosis signature (independently by gene and by combination of genes); Kaplan-Meier analysis for survival or events at 5 years by prognosis signature (independently by gene and by combination of genes) including p-values; univariate Cox or logistic regressions for survival or events at 5 years by prognosis signature (independently by gene and by combination of genes) including p-values; and multivariate Cox or logistic regressions for survival or events at 5 years by prognosis signature using individual genes (selected from Survival Analysis 3) or gene combination and incorporating significant clinical variables. References and additional statistical methodologies can be found in Van De Vijver, et al., NEJM, Vol. 347, No. 25 Dec. 19, 2002. and Tibshirani et al. 2002 PNAS 99(10) 6567-6572. Preferred analyses of expression results for the above identified gene products to identify individuals with good or poor prognosis include Kaplan-Meier analysis for survival, Cox-regression analyses or classification algorithms.

### Example 3

### Blood Samples

The prognosis of an individual with colon cancer can be determined based on the gene product expression of a peripheral blood sample. Peripheral blood samples are collected after consent from the individuals is obtained. For individuals with cancer, blood samples are often collected after surgery, and for individuals without cancer the blood can be collected at anytime.

Using the gene products of Table 2a and 2b and the methods of Example 2 and 3, blood samples from each individual are processed for analysis of gene products according to methods known by those of skill in the art. From each individual and control donor 10 ml of blood (in PaxGene tubes) is collected. RNA is extracted from blood samples by methods known by those of skill in the art, or by use of commercially available kits such as Qiagene RNA collection kits which utilize the Qiagene RNA collection procedure.

For analysis of RNA, an amplification step may be used to improve sensitivity using commercially available kits such as the Ovation™ System from Nugen™ (San Carlos, Calif.). Additionally, emerging amplification methodologies such as Whole Transcriptome Amplification (WTA) which does not demonstrate a 3′ bias as seen in other RNA detection methodologies may be utilized. Available WTA services and forthcoming commercially available WTA kits include Ribo-SPIA™ WTA from Nugen™ and the TransPlex™ Whole Transcriptome Amplification Kits from Rubicon Genomics (Ann Arbor, Mich.). See Nugen™ website nugentechnologies with the extension .com/technology-wt-spia.htm of the world wide web and Rubicon Genetics website rubicongenomics with the extension .com/web/OmniPlexWTAKits.html of the world wide web.

Blood samples from healthy individuals are used to determine a baseline level of expression for each of the gene products tested. All measurements of gene products are normalized against endogenous controls.

Specific gene products that can be used individually or in combination to detect and/or predict colon cancer for an individual include REGIV, NOX1, CEACAM5, TRIM15, REGIV-like protein, C20orf52, FAM3D, OLFM4, HOXB9, GAL4, CA1, UNQ511, MS4A8B, TSPAN1, CA1, ITLN1, TSPAN1, CYR61, CXCL12, C20orf52, DPEP1, SPP1, URCC, CEACAM6, AGR2, GDF15, SPON2, CCL20, C10orf35, SCD, TH1L, LCN2, MMP9, TYMS, TK1, DTYMK, CD44, NME1, MYBL2, TSPN6, HARS2, STAT6, GAL4, CA1, PIGR, REG3A, PACAP, NDRG1 and KRT20.

Specific gene products that are used to determine cancerous cells in the peripheral blood of an individual regularly include REGIV, NOX1, CEACAM5, TRIM15, REGIV-like protein, C20orf52, FAM3D, OLFM4, HOXB9, GAL4, CA1, UNQ511 and MS4A8B. In addition to these individual gene products, several multi-marker sets are also used to detect cancerous cells in an individual's peripheral blood. These multi-marker sets include REGIV, NOX1, CEACAM5, TRIM15, REGIV-like protein, C20orf52, FAM3D, OLFM4, HOXB9, GAL4, CA1, UNQ511, MS4A8B, TSPAN1, CA1, ITLN1, TSPAN1, CYR61, CXCL12, C20orf52, DPEP1, SPP1, URCC, CEACAM6, AGR2, GDF15, SPON2, CCL20, C10orf35, SCD, TH1L, LCN2, MMP9, TYMS, TK1, DTYMK, CD44, NME1, MYBL2, TSPN6, HARS2, STAT6, GAL4, CA1, PIGR, REG3A, PACAP, NDRG1 and KRT20.

### Example 4

### Lymph Nodes

The prognosis of an individual with colon cancer can be determined based on the gene product expression of a lymph node sample. Lymph node samples are collected through several methods. Individuals found to have colon cancer undergo an axillary lymph node dissection (lymph node is surgically removed) or they have a sentinel lymphandenectomy performed. In order to obtain non-cancerous lymph nodes, oftentimes individuals having surgeries such as a cholecystectomy or a tonsillectomy are asked to provide samples of their lymph nodes.

Using the gene products of Table 2a and 2b and the methods of Example 2 and 3, lymph node samples from each individual are processed for analysis of gene products according to methods known by those of skill in the art.

Lymph node samples from healthy individuals are used as controls and to determine a baseline level of expression for each of the gene products tested. All measurements of gene products are normalized against endogenous controls.

Specific gene products that can be used individually or in combination to detect and/or predict colon cancer for an individual include REGIV, NOX1, CEACAM5, TRIM15, REGIV-like protein, C20orf52, FAM3D, OLFM4, HOXB9, GAL4, CA1, UNQ511, MS4A8B, TSPAN1, CA1, ITLN1, TSPAN1, CYR61, CXCL12, C20orf52, DPEP1, SPP1, URCC, CEACAM6, AGR2, GDF15, SPON2, CCL20, C10orf35, SCD, TH1L, LCN2, MMP9, TYMS, TK1, DTYMK, CD44, NME1, MYBL2, TSPN6, HARS2, STAT6, GAL4, CA1, PIGR, REG3A, PACAP, NDRG1 and KRT20.

Specific gene products that are used to determine cancerous cells in the lymph nodes of an individual REGIV, NOX1, CEACAM5, TRIM15, REGIV-like protein, C20orf52, FAM3D, OLFM4, HOXB9, GAL4, CA1, UNQ511 and MS4A8B. In addition to these individual gene products, several multi-marker sets are also used to detect cancerous cells in an individual's lymph nodes. These multi-marker sets include REGIV, NOX1, CEACAM5, TRIM15, REGIV-like protein, C20orf52, FAM3D, OLFM4, HOXB9, GAL4, CA1, UNQ511, MS4A8B, TSPAN1, CA1, ITLN1, TSPAN1, CYR61, CXCL12, C20orf52, DPEP1, SPP1, URCC, CEACAM6, AGR2, GDF15, SPON2, CCL20, C10orf35, SCD, TH1L, LCN2, MMP9, TYMS, TK1, DTYMK, CD44, NME1, MYBL2, TSPN6, HARS2, STAT6, GAL4, CA1, PIGR, REG3A, PACAP, NDRG1 and KRT20.

### Example 5

### Fecal Samples

The prognosis of an individual with colon cancer can be determined based on the gene product expression of a fecal sample. Fecal samples are collected through several methods know by those of skill in the art. Individuals with or suspected of having colon cancer may provide a fecal sample for evaluation.

Using the gene products of Table 2a and 2b and the methods of Example 2 and 3, fecal samples from each individual are processed for analysis of gene products according to methods known by those of skill in the art. See Kanaoka, et al., Gastroenterology, Vol. 127, No. 2 December, 2004.

Fecal samples from healthy individuals are used as controls and to determine a baseline level of expression for each of the gene products tested. All measurements of gene products are normalized against endogenous controls.

Specific gene products that can be used individually or in combination to detect and/or predict colon cancer for an individual include REGIV, NOX1, CEACAM5, TRIM15, REGIV-like protein, C20orf52, FAM3D, OLFM4, HOXB9, GAL4, CA1, UNQ511, MS4A8B, TSPAN1, CA1, ITLN1, TSPAN1, CYR61, CXCL12, C20orf52, DPEP1, SPP1, URCC, CEACAM6, AGR2, GDF15, SPON2, CCL20, C10orf35, SCD, TH1L, LCN2, MMP9, TYMS, TK1, DTYMK, CD44, NME1, MYBL2, TSPN6, HARS2, STAT6, GAL4, CA1, PIGR, REG3A, PACAP, NDRG1 and KRT20.

Specific gene products that are used to determine cancerous cells in the feces of an individual include REGIV, NOX1, CEACAM5, TRIM15, REGIV-like protein, C20orf52, FAM3D, OLFM4, HOXB9, GAL4, CA1, UNQ511 and MS4A8B. In addition to these individual gene products, several multi-marker sets are also used to detect cancerous cells in an individual's feces. These multi-marker sets include REGIV, NOX1, CEACAM5, TRIM15, REGIV-like protein, C20orf52, FAM3D, OLFM4, HOXB9, GAL4, CA1, UNQ511, MS4A8B, TSPAN1, CA1, ITLN1, TSPAN1, CYR61, CXCL12, C20orf52, DPEP1, SPP1, URCC, CEACAM6, AGR2, GDF15, SPON2, CCL20, C10orf5, SCD, TH1L, LCN2, MMP9, TYMS, TK1, DTYMK, CD44, NME1, MYBL2, TSPN6, HARS2, STAT6, GAL4, CA1, PIGR, REG3A, PACAP, NDRG1 and KRT20.

1. A method for determining the prognosis for an individual having colon cancer comprising: determining an expression level of a plurality of gene products of genes in Table 2a in a sample from an individual relative to a control, wherein differential expression of the plurality of gene products relative to a control is indicative of the individual's prognosis.

2. The method of claim 1 further comprising determining an expression level of a plurality of gene products of genes in Table 2b in the sample from the individual relative to the control.

3. The method of claim 1 wherein the plurality of gene products comprises at least two gene products.

4. The method of claim 1 wherein the plurality of gene products comprises at least four gene products.

5. The method of claim 1 wherein the plurality of gene products comprises at least six gene products.

6. The method of claim 1 wherein the plurality of gene products comprises at least eight gene products.

7. The method of claim 2 wherein the gene products are selected from the group comprising CA1, ITLN1, TSPAN1, CYR61, CXCL12, C20orf52, DPEP1, REGIV, NOX1, CEACAM5, FAM3D, OLFM4, HOXB9, SPP1, URCC, CEACAM6, AGR2, GDF15, SPON2, CCL20, C10orf35, SCD, TH1L, LCN2, MMP9, TYMS, TK1, DTYMK, CD44, NME1, MYBL2, TSPN6, HARS2, STAT6, GAL4, CA1, PIGR, REG3A, PACAP, NDRG1 and KRT20.

8. The method of claim 7 wherein 5 to 15 gene products are selected from the group comprising CA1, ITLN1, TSPAN1, CYR61, CXCL12, C20orf52, DPEP1, REGIV, NOX1, CEACAM5, FAM3D, OLFM4, HOXB9, SPP1, URCC, CEACAM6, AGR2, GDF15, SPON2, CCL20, C10orf35, SCD, TH1L, LCN2, MMP9, TYMS, TK1, DTYMK, CD44, NME1, MYBL2, TSPN6, HARS2, STAT6, GAL4, CA1, PIGR, REG3A, PACAP, NDRG1 and KRT20.

9. The method of claim 7 wherein over-expression of a gene product selected from the group comprising CA1, ITLN1, TSPAN1, CYR61 and CXCL12 is indicative of a good prognosis.

10. The method of claim 7 wherein under-expression of a gene product selected from the group comprising C20orf52 and DPEP1 is indicative of a good prognosis.

11. The method of claim 7 wherein over-expression of a gene product selected from the group comprising REGIV, NOX1, CEACAM5, C20orf52, FAM3D, OLFM4, HOXB9, SPP1, URCC, CEACAM6, AGR2, GDF15, CCL20, C10orf35, SCD, TH1L, LCN2, MMP9, TYMS, TK1, DTYMK, CD44, NME1, MYBL2, DPEP1, TSPN6, HARS2 and STAT6 is indicative of a poor prognosis.

12. The method of claim 7 wherein under-expression of a gene product selected from the group comprising GAL4, CA1, PIGR, REG3A, PACAP, CYR61, NDRG1, CXCL12 and KRT20 is indicative of a poor prognosis.

13. The method of claim 2 where in the gene product is a RNA.

14. The method of claim 13 wherein the gene product expression level is determined by quantitative PCR.

15. The method of claim 13 wherein the gene product expression level is determined by microarray analysis.

16. The method of claim 1 wherein the gene product is a polypeptide.

17. The method of claim 16 wherein the gene product expression is determined by an assay comprising one or more antibodies.

18. The method of claim 2 wherein the sample is selected from the group comprising tissues, lymph nodes, cells and bodily fluids.

19. The method of claim 18 wherein the tissues, lymph nodes or cells are from a fixed, waxed embedded specimen from said individual.

20. The method of claim 18 wherein the tissues, lymph nodes or cells are from a fresh frozen specimen from said individual.

21. A method for improving the prognosis for an individual comprising modulating expression levels or activity of a plurality of gene products of Table 2a.

22. The method of claim 21 wherein the plurality of gene products comprises at least two gene products.

23. The method of claim 21 wherein the plurality of gene products comprises at least four gene products.

24. The method of claim 21 wherein the plurality of gene products comprises at least six gene products.

25. The method of claim 21 wherein the plurality of gene products comprises at least eight gene products.

26. The method of claim 21 wherein modulating expression levels or activity of gene products comprises increasing expression levels or activity of gene products whose over-expression is associated with a good prognosis.

27. The method of claim 21 wherein modulating expression levels or activity of gene products comprises decreasing expression levels or activity of gene products whose under-expression is associated with a good prognosis.

28. The method of claim 21 wherein modulating expression levels or activity of gene products comprises decreasing expression levels or activity of gene products whose over-expression is associated with a poor prognosis.

29. The method of claim 21 wherein modulating expression levels or activity of gene products comprises increasing expression levels or activity of gene products whose under-expression is associated with a poor prognosis.

30. The method of claim 21 wherein an agonist or antagonist for a gene product of Table 2a is administered to the individual to improve the prognosis of the individual.

31. An isolated nucleic acid molecule comprising:
(a) a nucleic acid molecule consisting essentially of a nucleic acid sequence of Table 7;
(b) a nucleic acid molecule that selectively hybridizes to the nucleic acid molecule of (a); or
(c) a nucleic acid molecule having at least 95% sequence identity to the nucleic acid molecule of (a).

32. The nucleic acid molecule according to claim 31, wherein the nucleic acid molecule is cDNA.

33. The nucleic acid molecule according to claim 31, wherein the nucleic acid molecule is genomic DNA.

34. The nucleic acid molecule according to claim 31, wherein the nucleic acid molecule is RNA.

35. The nucleic acid molecule according to claim 31, wherein the nucleic acid molecule is a mammalian nucleic acid molecule.

36. The nucleic acid molecule according to claim 35, wherein the nucleic acid molecule is a human nucleic acid molecule.

37. A set of three isolated nucleic acid molecules wherein:
(a) each nucleic acid molecule consists essentially of a nucleic acid sequence encoding a portion of gene product described in Table 2a or Table 2b and
(i) the first nucleic acid molecule is a forward primer 15 to 30 base pairs in length;
(ii) the second nucleic acid molecule is a reverse primer 15 to 30 base pairs in length; and
(iii) the third nucleic acid molecule is a probe 15-30 base pairs in length; such that the forward primer and reverse primer produce an amplicon detectable by the probe wherein the amplicon bridges two exons and is 60 to 100 base pairs in length;
(b) each nucleic acid molecule selectively hybridizes to one of the three nucleic acid molecules of (a); or
(c) each nucleic acid molecule has at least 95% sequence identity to the one of the three nucleic acid molecules of (a).

38. The set of nucleic acid molecules of claim 37 wherein the amplicon is contained in one exon.

39. The set of nucleic acid molecules of claim 37 wherein the amplicon bridges two exons.

40. The set of nucleic acid molecules of claim 37 wherein the amplicon bridges at least two exons.

41. A method for determining the presence of a gene product of Table 2a or Table 2b in a sample, comprising the steps of:
(a) contacting the sample with a nucleic acid molecule of Table 7 under conditions in which the nucleic acid molecule will selectively hybridize to a gene product of Table 2a or Table 2b; and
(b) detecting hybridization of the nucleic acid molecule to a gene product of Table 2a or Table 2b in the sample, wherein the detection of the hybridization indicates the presence of a gene product of Table 2a or Table 2b in the sample.

42. A method for determining the presence of a cancer specific protein in a sample, comprising the steps of:
(a) contacting the sample with a suitable reagent under conditions in which the reagent will selectively interact with the cancer specific protein comprising an amino acid sequence with at least 95% sequence identity to a polypeptide encoded by a gene product in Table 2a or Table 2b; and
(b) detecting the interaction of the reagent with any cancer specific protein in the sample, wherein the detection of binding indicates the presence of cancer specific protein in the sample.

42. (canceled)

43. A kit for detecting a risk of cancer or presence of cancer in an individual, said kit comprising a means for determining the presence of:
(a) a nucleic acid molecule consisting essentially of a nucleic acid sequence that encodes an amino acid sequence of the polypeptide encoded by a gene product in Table 2a or Table 2b;
(b) a nucleic acid molecule consisting essentially of a nucleic acid sequence of a gene product in Table 2a or Table 2b;
(c) a nucleic acid molecule consisting essentially of a nucleic acid sequence of Table 7;
(d) a nucleic acid molecule that selectively hybridizes to the nucleic acid molecule of (a), (b) or (c);
(e) a nucleic acid molecule having at least 95% sequence identity to the nucleic acid molecule of (a), (b) or (c);
(f) a polypeptide comprising an amino acid sequence with at least 95% sequence identity to the polypeptide encoded by a gene product in Table 2a; or
(g) a polypeptide comprising an amino acid sequence encoded by a nucleic acid molecule having at least 95% sequence identity to a nucleic acid molecule comprising a nucleic acid sequence of a nucleic acid molecule consisting essentially of a nucleic acid sequence of a gene product of Table 2a or Table 2b.

44. A method of treating an individual with colon cancer, comprising the step of administering a composition consisting of:
(a) a nucleic acid molecule consisting essentially of a nucleic acid sequence that encodes an amino acid sequence of the polypeptide encoded by a gene product in Table 2a or Table 2b;
(b) a nucleic acid molecule consisting essentially of a nucleic acid sequence of a gene product in Table 2a or Table 2b;
(c) a nucleic acid molecule consisting essentially of a nucleic acid sequence of Table 7;
(d) a nucleic acid molecule that selectively hybridizes to the nucleic acid molecule of (a), (b) or (c);
(e) a nucleic acid molecule having at least 95% sequence identity to the nucleic acid molecule of (a), (b) or (c);
(f) a polypeptide comprising an amino acid sequence with at least 95% sequence identity to the polypeptide encoded by a gene product in Table 2a;
(g) a polypeptide comprising an amino acid sequence encoded by a nucleic acid molecule having at least 95% sequence identity to a nucleic acid molecule comprising a nucleic acid sequence of a nucleic acid molecule consisting essentially of a nucleic acid sequence of a gene product of Table 2a; or
(h) an appropriate agonist or antagonist for a gene product of Table 2a or Table 2b to an individual in need thereof, wherein said administration induces an immune response against the colon cancer cell expressing the nucleic acid molecule or polypeptide.

45. A method for diagnosing or monitoring the presence and metastases of colon cancer in an individual, comprising the steps of:
(a) determining an amount of:
(i) a nucleic acid molecule consisting essentially of a nucleic acid sequence that encodes an amino acid sequence of a gene product in Table 2a or Table 2b;
(ii) a nucleic acid molecule consisting essentially of a nucleic acid sequence of a gene product in Table 2a or Table 2b;
(iii) a nucleic acid molecule consisting essentially of a nucleic acid sequence of Table 7;
(iv) a nucleic acid molecule that selectively hybridizes to the nucleic acid molecule of (i), (ii) or (iii);
(v) a nucleic acid molecule having at least 95% sequence identity to the nucleic acid molecule of (i), (ii) or (iii);
(vi) a polypeptide comprising an amino acid sequence with at least 95% sequence identity to the polypeptide encoded by a gene product in Table 2a or Table 2b; or
(vii) a polypeptide comprising an amino acid sequence encoded by a nucleic acid molecule having at least 95% sequence identity to a nucleic acid molecule consisting essentially of a nucleic acid sequence of a gene product of Table 2a or Table 2b; and
(b) comparing the amount of the determined nucleic acid molecule or the polypeptide in the sample of the individual to the amount of the cancer specific marker in a normal control; wherein a difference in the amount of the nucleic acid molecule or the polypeptide in the sample compared to the amount of the nucleic acid molecule or the polypeptide in the normal control is associated with the presence of colon cancer.


SEQUENCE LISTING (464 sequences)

SEQ ID NO: 1 - DNA, 75 bp, Artificial sequence
  Synthetic
ggaacaagcactgtgctgagatgagctccaataacaactttttaacttggagcagcaacgaatgcaacaagcgcc

SEQ ID NO: 2 - DNA, 22 bp, Artificial sequence
  Synthetic
ggaacaagcactgtgctgagat

SEQ ID NO: 3 - DNA, 17 bp, Artificial sequence
  Synthetic
ggcgcttgttgcattcg

SEQ ID NO: 4 - DNA, 34 bp, Artificial sequence
  Synthetic
agctccaataacaactttttaacttggagcagca

SEQ ID NO: 5 - DNA, 75 bp, Artificial sequence
  Synthetic
ccgctcccagcagaaggttgtgattaccaaggttgttatgcacccatccaaagttttggaattgcagatgaacaa

SEQ ID NO: 6 - DNA, 18 bp, Artificial sequence
  Synthetic
ccgctcccagcagaaggt

SEQ ID NO: 7 - DNA, 24 bp, Artificial sequence
  Synthetic
ttgttcatctgcaattccaaaact

SEQ ID NO: 8 - DNA, 30 bp, Artificial sequence
  Synthetic
tgattaccaaggttgttatgcacccatcca

SEQ ID NO: 9 - DNA, 84 bp, Artificial sequence
  Synthetic
ggacctatgcctgttttgtctctaacttggctactggccgcaataattccatagtcaagagcatcacagtctctgacagttgtt

SEQ ID NO: 10 - DNA, 25 bp, Artificial sequence
  Synthetic
ggacctatgcctgttttgtctctaa

SEQ ID NO: 11 - DNA, 25 bp, Artificial sequence
  Synthetic
aacaactgtcagagactgtgatgct

SEQ ID NO: 12 - DNA, 29 bp, Artificial sequence
  Synthetic
ttggctactggccgcaataattccatagt

SEQ ID NO: 13 - DNA, 94 bp, Artificial sequence
  Synthetic
tggaaatagattcaggggtcatcactctggaccctcagaccgccagccggagcctggttctctcggaagacaggaagtcagtgaggtacacccg

SEQ ID NO: 14 - DNA, 22 bp, Artificial sequence
  Synthetic
tggaaatagattcaggggtcat

SEQ ID NO: 15 - DNA, 21 bp, Artificial sequence
  Synthetic
cgggtgtacctcactgacttc

SEQ ID NO: 16 - DNA, 25 bp, Artificial sequence
  Synthetic
tgtcttccgagagaaccaggctccg

SEQ ID NO: 17 - DNA, 86 bp, Artificial sequence
  Synthetic
ggtgaagtcagccaacaaagggttgatggaagctgagaaggatacagcagagatgacaaccaaaatacttaaccatcgggacagcg

SEQ ID NO: 18 - DNA, 21 bp, Artificial sequence
  Synthetic
ggtgaagtcagccaacaaagg

SEQ ID NO: 19 - DNA, 20 bp, Artificial sequence
  Synthetic
cgctgtcccgatggttaagt

SEQ ID NO: 20 - DNA, 31 bp, Artificial sequence
  Synthetic
ttgatggaagctgagaaggatacagcagaga

SEQ ID NO: 21 - DNA, 71 bp, Artificial sequence
  Synthetic
gagacccctggaagctgatggaggcaacgtgagaagcacatggacatccgtccttgagcttgagaggcaga

SEQ ID NO: 22 - DNA, 20 bp, Artificial sequence
  Synthetic
gagacccctggaagctgatg

SEQ ID NO: 23 - DNA, 21 bp, Artificial sequence
  Synthetic
tctgcctctcaagctcaagga

SEQ ID NO: 24 - DNA, 27 bp, Artificial sequence
  Synthetic
aggcaacgtgagaagcacatggacatc

SEQ ID NO: 25 - DNA, 89 bp, Artificial sequence
  Synthetic
ttcctatcccccgcagatgccggtggccgtgggtccctacggacagtcccagccaagctgcttcgaccgtgtcaaaatgggcttcgtga

SEQ ID NO: 26 - DNA, 19 bp, Artificial sequence
  Synthetic
ttcctatcccccgcagatg

SEQ ID NO: 27 - DNA, 20 bp, Artificial sequence
  Synthetic
tcacgaagcccattttgaca

SEQ ID NO: 28 - DNA, 24 bp, Artificial sequence
  Synthetic
tccctacggacagtcccagccaag

SEQ ID NO: 29 - DNA, 83 bp, Artificial sequence
  Synthetic
tgggcttcgtgatgggttgcgccgtgggcatggcggccggggcgctcttcggcaccttttcctgtctcaggatcggaatgcgg

SEQ ID NO: 30 - DNA, 18 bp, Artificial sequence
  Synthetic
tgggcttcgtgatgggtt

SEQ ID NO: 31 - DNA, 18 bp, Artificial sequence
  Synthetic
ccgcattccgatcctgag

SEQ ID NO: 32 - DNA, 23 bp, Artificial sequence
  Synthetic
acaggaaaaggtgccgaagagcg

SEQ ID NO: 33 - DNA, 84 bp, Artificial sequence
  Synthetic
ccccaagccacccttcacctggacaggatgagagtgtcaggtgtgcttcgcctcctggccctcatctttgccatagtcacgaca

SEQ ID NO: 34 - DNA, 17 bp, Artificial sequence
  Synthetic
ccccaagccacccttca

SEQ ID NO: 35 - DNA, 23 bp, Artificial sequence
  Synthetic
tgtcgtgactatggcaaagatga

SEQ ID NO: 36 - DNA, 28 bp, Artificial sequence
  Synthetic
acaggatgagagtgtcaggtgtgcttcg

SEQ ID NO: 37 - DNA, 72 bp, Artificial sequence
  Synthetic
ctctgcccaggtggaagctgaccccaagccacccttcacctggacaggatgagagtgtcaggtgtgcttcgc

SEQ ID NO: 38 - DNA, 19 bp, Artificial sequence
  Synthetic
ctctgcccaggtggaagct

SEQ ID NO: 39 - DNA, 21 bp, Artificial sequence
  Synthetic
gcgaagcacacctgacactct

SEQ ID NO: 40 - DNA, 23 bp, Artificial sequence
  Synthetic
accccaagccacccttcacctgg

SEQ ID NO: 41 - DNA, 72 bp, Artificial sequence
  Synthetic
gaaccctcccacagcacatcctacccggaagaccagcctcagagggtccttctggaaccagctgtctgtgga

SEQ ID NO: 42 - DNA, 20 bp, Artificial sequence
  Synthetic
gaaccctcccacagcacatc

SEQ ID NO: 43 - DNA, 20 bp, Artificial sequence
  Synthetic
tccacagacagctggttcca

SEQ ID NO: 44 - DNA, 25 bp, Artificial sequence
  Synthetic
tacccggaagaccagcctcagaggg

SEQ ID NO: 45 - DNA, 73 bp, Artificial sequence
  Synthetic
acccctgtcgtccaccctcctcccactccagggagctgtggtcatggtggtgtggtgaacatcagcaaaccgt

SEQ ID NO: 46 - DNA, 18 bp, Artificial sequence
  Synthetic
acccctgtcgtccaccct

SEQ ID NO: 47 - DNA, 20 bp, Artificial sequence
  Synthetic
acggtttgctgatgttcacc

SEQ ID NO: 48 - DNA, 25 bp, Artificial sequence
  Synthetic
acaccaccatgaccacagctccctg

SEQ ID NO: 49 - DNA, 85 bp, Artificial sequence
  Synthetic
cttcaggtgctaaacacttggtataccaagcagtataaaccatctgcttctaacgccttcatggtatgtggggttctgtatgcca

SEQ ID NO: 50 - DNA, 25 bp, Artificial sequence
  Synthetic
cttcaggtgctaaacacttggtata

SEQ ID NO: 51 - DNA, 22 bp, Artificial sequence
  Synthetic
tggcatacagaaccccacatac

SEQ ID NO: 52 - DNA, 31 bp, Artificial sequence
  Synthetic
tgaaggcgttagaagcagatggtttatactg

SEQ ID NO: 53 - DNA, 90 bp, Artificial sequence
  Synthetic
ccacacttcaggtgctaaacacttggtataccaagcagtataaaccatctgcttctaacgccttcatggtatgtggggttctgtatgcca

SEQ ID NO: 54 - DNA, 23 bp, Artificial sequence
  Synthetic
ccacacttcaggtgctaaacact

SEQ ID NO: 55 - DNA, 22 bp, Artificial sequence
  Synthetic
tggcatacagaaccccacatac

SEQ ID NO: 56 - DNA, 32 bp, Artificial sequence
  Synthetic
atgaaggcgttagaagcagatggtttatactg

SEQ ID NO: 57 - DNA, 74 bp, Artificial sequence
  Synthetic
cgcgcttctgggaaacagtcccgcgtgcaaaggaaaggggcaaaatggcacctaagcatcagatggaagcttac

SEQ ID NO: 58 - DNA, 17 bp, Artificial sequence
  Synthetic
cgcgcttctgggaaaca

SEQ ID NO: 59 - DNA, 25 bp, Artificial sequence
  Synthetic
gtaagcttccatctgatgcttaggt

SEQ ID NO: 60 - DNA, 21 bp, Artificial sequence
  Synthetic
tcccgcgtgcaaaggaaaggg

SEQ ID NO: 61 - DNA, 81 bp, Artificial sequence
  Synthetic
ggacaataaaatttgcgaaggaagcgaggacaaagagaggccggatcaaaccaacccctccgccaactggctgcacgctcg

SEQ ID NO: 62 - DNA, 23 bp, Artificial sequence
  Synthetic
ggacaataaaatttgcgaaggaa

SEQ ID NO: 63 - DNA, 17 bp, Artificial sequence
  Synthetic
cgagcgtgcagccagtt

SEQ ID NO: 64 - DNA, 23 bp, Artificial sequence
  Synthetic
agaggccggatcaaaccaacccc

SEQ ID NO: 65 - DNA, 82 bp, Artificial sequence
  Synthetic
aacccgcctgtgccatatttcgggaggctgcaaggagggctcacagctcgaagaaccatcatcatcaagggctatgtgcctc

SEQ ID NO: 66 - DNA, 19 bp, Artificial sequence
  Synthetic
aacccgcctgtgccatatt

SEQ ID NO: 67 - DNA, 21 bp, Artificial sequence
  Synthetic
gaggcacatagcccttgatga

SEQ ID NO: 68 - DNA, 27 bp, Artificial sequence
  Synthetic
aggagggctcacagctcgaagaaccat

SEQ ID NO: 69 - DNA, 77 bp, Artificial sequence
  Synthetic
gtactccagccttgctgaagctgcctcaaaggctgatggtttggcagttattggtgttttgatgaaggttggtgagg

SEQ ID NO: 70 - DNA, 22 bp, Artificial sequence
  Synthetic
gtactccagccttgctgaagct

SEQ ID NO: 71 - DNA, 23 bp, Artificial sequence
  Synthetic
cctcaccaaccttcatcaaaaca

SEQ ID NO: 72 - DNA, 29 bp, Artificial sequence
  Synthetic
cctcaaaggctgatggtttggcagttatt

SEQ ID NO: 73 - DNA, 77 bp, Artificial sequence
  Synthetic
agtctaagagtctcgtgctgaaaggctgttccaacgtcagtaacgccacctgtcagttcctgtctggtgaaaacaag

SEQ ID NO: 74 - DNA, 25 bp, Artificial sequence
  Synthetic
agtctaagagtctcgtgctgaaagg

SEQ ID NO: 75 - DNA, 24 bp, Artificial sequence
  Synthetic
cttgttttcaccagacaggaactg

SEQ ID NO: 76 - DNA, 26 bp, Artificial sequence
  Synthetic
tgttccaacgtcagtaacgccacctg

SEQ ID NO: 77 - DNA, 78 bp, Artificial sequence
  Synthetic
agcccattttgccaaccaagcaaacaccacaaccaatatgtctgtcctggttattccaaatatgtatgaaagcaaccc

SEQ ID NO: 78 - DNA, 18 bp, Artificial sequence
  Synthetic
agcccattttgccaacca

SEQ ID NO: 79 - DNA, 24 bp, Artificial sequence
  Synthetic
gggttgctttcatacatatttgga

SEQ ID NO: 80 - DNA, 30 bp, Artificial sequence
  Synthetic
caaacaccacaaccaatatgtctgtcctgg

SEQ ID NO: 81 - DNA, 78 bp, Artificial sequence
  Synthetic
ggcttctactggtgtctgaccaacggcgatactctctggaggaccaccgtggagatcaagattatcgaaggagaacca

SEQ ID NO: 82 - DNA, 23 bp, Artificial sequence
  Synthetic
ggcttctactggtgtctgaccaa

SEQ ID NO: 83 - DNA, 25 bp, Artificial sequence
  Synthetic
tggttctccttcgataatcttgatc

SEQ ID NO: 84 - DNA, 26 bp, Artificial sequence
  Synthetic
cgatactctctggaggaccaccgtgg

SEQ ID NO: 85 - DNA, 75 bp, Artificial sequence
  Synthetic
cgccttgtgtgctggaatgagggtcaccggatgtaacactgagcaccactgcattggtggaggaggatactttcc

SEQ ID NO: 86 - DNA, 19 bp, Artificial sequence
  Synthetic
cgccttgtgtgctggaatg

SEQ ID NO: 87 - DNA, 22 bp, Artificial sequence
  Synthetic
ggaaagtatcctcctccaccaa

SEQ ID NO: 88 - DNA, 26 bp, Artificial sequence
  Synthetic
ccggatgtaacactgagcaccactgc

SEQ ID NO: 89 - DNA, 81 bp, Artificial sequence
  Synthetic
gcagttggagtcatactcttcatcacagatctaagtattccccacccatatgcctaccccgactattatccttacgcctgg

SEQ ID NO: 90 - DNA, 24 bp, Artificial sequence
  Synthetic
gcagttggagtcatactcttcatc

SEQ ID NO: 91 - DNA, 22 bp, Artificial sequence
  Synthetic
ccaggcgtaaggataatagtcg

SEQ ID NO: 92 - DNA, 29 bp, Artificial sequence
  Synthetic
cagatctaagtattccccacccatatgcc

SEQ ID NO: 93 - DNA, 72 bp, Artificial sequence
  Synthetic
ccaacacagccaatgaaacctgcaccaagcaaaaggctcacgaccaaaaagtagagggttgcttcaatcagc

SEQ ID NO: 94 - DNA, 21 bp, Artificial sequence
  Synthetic
ccaacacagccaatgaaacct

SEQ ID NO: 95 - DNA, 24 bp, Artificial sequence
  Synthetic
gctgattgaagcaaccctctactt

SEQ ID NO: 96 - DNA, 25 bp, Artificial sequence
  Synthetic
caccaagcaaaaggctcacgaccaa

SEQ ID NO: 97 - DNA, 71 bp, Artificial sequence
  Synthetic
ggtaccaggagctgaggagtgtgagcgtgccgtcatcgactttgtggctttccaggacatctccatcaaga

SEQ ID NO: 98 - DNA, 22 bp, Artificial sequence
  Synthetic
ggtaccaggagctgaggagtgt

SEQ ID NO: 99 - DNA, 22 bp, Artificial sequence
  Synthetic
tcttgatggagatgtcctggaa

SEQ ID NO: 100 - DNA, 24 bp, Artificial sequence
  Synthetic
agcgtgccgtcatcgactttgtgg

SEQ ID NO: 101 - DNA, 86 bp, Artificial sequence
  Synthetic
tgtgcacagaggtaggagaaaaaattgcccttagccgaagagttgaaaaacactggcgtttaattggttggggtcagataagaaga

SEQ ID NO: 102 - DNA, 24 bp, Artificial sequence
  Synthetic
tgtgcacagaggtaggagaaaaaa

SEQ ID NO: 103 - DNA, 23 bp, Artificial sequence
  Synthetic
tcttcttatctgaccccaaccaa

SEQ ID NO: 104 - DNA, 30 bp, Artificial sequence
  Synthetic
tgcccttagccgaagagttgaaaaacactg

SEQ ID NO: 105 - DNA, 81 bp, Artificial sequence
  Synthetic
gcagctgctttgtcaccaactccggggactatgagattctgtactgggacccggctacctgtaagcagatcaccagtgcgg

SEQ ID NO: 106 - DNA, 21 bp, Artificial sequence
  Synthetic
gcagctgctttgtcaccaact

SEQ ID NO: 107 - DNA, 20 bp, Artificial sequence
  Synthetic
ccgcactggtgatctgctta

SEQ ID NO: 108 - DNA, 30 bp, Artificial sequence
  Synthetic
atgagattctgtactgggacccggctacct

SEQ ID NO: 109 - DNA, 86 bp, Artificial sequence
  Synthetic
gccagttgcagccttctcagccaaacgccgaccaaggaaaactcactaccatgagaattgcagtgatttgcttttgcctcctaggc

SEQ ID NO: 110 - DNA, 19 bp, Artificial sequence
  Synthetic
gccagttgcagccttctca

SEQ ID NO: 111 - DNA, 20 bp, Artificial sequence
  Synthetic
gcctaggaggcaaaagcaaa

SEQ ID NO: 112 - DNA, 25 bp, Artificial sequence
  Synthetic
caaacgccgaccaaggaaaactcac

SEQ ID NO: 113 - DNA, 78 bp, Artificial sequence
  Synthetic
ggttctctatcaacggccacttctacaatcataagacctccgtgtttactccagcctatggatccgtgaccaatgtga

SEQ ID NO: 114 - DNA, 22 bp, Artificial sequence
  Synthetic
ggttctctatcaacggccactt

SEQ ID NO: 115 - DNA, 20 bp, Artificial sequence
  Synthetic
tcacattggtcacggatcca

SEQ ID NO: 116 - DNA, 33 bp, Artificial sequence
  Synthetic
tacaatcataagacctccgtgtttactccagcc

SEQ ID NO: 117 - DNA, 76 bp, Artificial sequence
  Synthetic
acgtccagcccccagttacgcctgcctcacaccttgcgatgcgccaacatcaccatcattgagcaccagaagtgtg

SEQ ID NO: 118 - DNA, 19 bp, Artificial sequence
  Synthetic
acgtccagcccccagttac

SEQ ID NO: 119 - DNA, 23 bp, Artificial sequence
  Synthetic
cacacttctggtgctcaatgatg

SEQ ID NO: 120 - DNA, 22 bp, Artificial sequence
  Synthetic
tcacaccttgcgatgcgccaac

SEQ ID NO: 121 - DNA, 74 bp, Artificial sequence
  Synthetic
ccagaaagcaagggcagatggcagtgggtctgttgatgagagaactttaagggcccaatcagtccctgggcacc

SEQ ID NO: 122 - DNA, 20 bp, Artificial sequence
  Synthetic
ccagaaagcaagggcagatg

SEQ ID NO: 123 - DNA, 19 bp, Artificial sequence
  Synthetic
ggtgcccagggactgattg

SEQ ID NO: 124 - DNA, 32 bp, Artificial sequence
  Synthetic
agtgggtctgttgatgagagaactttaagggc

SEQ ID NO: 125 - DNA, 89 bp, Artificial sequence
  Synthetic
tgtggacccctggttacatcagcatcggacttgccccctctgcatgttcaacatcacagagggagattcattttcccagtccctgggac

SEQ ID NO: 126 - DNA, 21 bp, Artificial sequence
  Synthetic
tgtggacccctggttacatca

SEQ ID NO: 127 - DNA, 20 bp, Artificial sequence
  Synthetic
gtcccagggactgggaaaat

SEQ ID NO: 128 - DNA, 23 bp, Artificial sequence
  Synthetic
cggacttgccccctctgcatgtt

SEQ ID NO: 129 - DNA, 86 bp, Artificial sequence
  Synthetic
ggccaactctttttactccttcattttccagtagttgggcagtgctggcactcctgtcacttttaatgaaaacggagatgctcctg

SEQ ID NO: 130 - DNA, 25 bp, Artificial sequence
  Synthetic
ggccaactctttttactccttcatt

SEQ ID NO: 131 - DNA, 22 bp, Artificial sequence
  Synthetic
caggagcatctccgttttcatt

SEQ ID NO: 132 - DNA, 24 bp, Artificial sequence
  Synthetic
tccagtagttgggcagtgctggca

SEQ ID NO: 133 - DNA, 86 bp, Artificial sequence
  Synthetic
atgtgccaagcccataactcagccactggcctcaataggaccacagtcacgatgatcacagtctctggaagtgctcctgtcctctc

SEQ ID NO: 134 - DNA, 21 bp, Artificial sequence
  Synthetic
atgtgccaagcccataactca

SEQ ID NO: 135 - DNA, 21 bp, Artificial sequence
  Synthetic
gagaggacaggagcacttcca

SEQ ID NO: 136 - DNA, 28 bp, Artificial sequence
  Synthetic
ccactggcctcaataggaccacagtcac

SEQ ID NO: 137 - DNA, 79 bp, Artificial sequence
  Synthetic
cagaaattggcagagcagtttgtcctcctcaatctggtttatgaaacaactgacaaacacctttctcctgatggccagt

SEQ ID NO: 138 - DNA, 22 bp, Artificial sequence
  Synthetic
cagaaattggcagagcagtttg

SEQ ID NO: 139 - DNA, 21 bp, Artificial sequence
  Synthetic
actggccatcaggagaaaggt

SEQ ID NO: 140 - DNA, 34 bp, Artificial sequence
  Synthetic
cctcctcaatctggtttatgaaacaactgacaaa

SEQ ID NO: 141 - DNA, 66 bp, Artificial sequence
  Synthetic
cactccgaagactccagattccgagagttgcggaaacgctacgaggacctgctaaccaggctgcgg

SEQ ID NO: 142 - DNA, 22 bp, Artificial sequence
  Synthetic
cactccgaagactccagattcc

SEQ ID NO: 143 - DNA, 17 bp, Artificial sequence
  Synthetic
ccgcagcctggttagca

SEQ ID NO: 144 - DNA, 24 bp, Artificial sequence
  Synthetic
agagttgcggaaacgctacgagga

SEQ ID NO: 145 - DNA, 67 bp, Artificial sequence
  Synthetic
ctcccgccccagtcctgcccagcagggacaatgagattgtagacagcgcctcagttccagaaacgcc

SEQ ID NO: 146 - DNA, 16 bp, Artificial sequence
  Synthetic
ctcccgccccagtcct

SEQ ID NO: 147 - DNA, 19 bp, Artificial sequence
  Synthetic
ggcgtttctggaactgagg

SEQ ID NO: 148 - DNA, 27 bp, Artificial sequence
  Synthetic
cgctgtctacaatctcattgtccctgc

SEQ ID NO: 149 - DNA, 86 bp, Artificial sequence
  Synthetic
gtgtatcttggatgctgctttcctgcctcatgctgctgtctcaggttcaaggtgaagaaccccagagggaactgccctctgcacgg

SEQ ID NO: 150 - DNA, 23 bp, Artificial sequence
  Synthetic
gtgtatcttggatgctgctttcc

SEQ ID NO: 151 - DNA, 18 bp, Artificial sequence
  Synthetic
ccgtgcagagggcagttc

SEQ ID NO: 152 - DNA, 28 bp, Artificial sequence
  Synthetic
catgctgctgtctcaggttcaaggtgaa

SEQ ID NO: 153 - DNA, 86 bp, Artificial sequence
  Synthetic
cagctaccatcatggtgaccaatccacactaccagggccaaatcctaactatgcttctgagaagccttcaacagccttctgcctcc

SEQ ID NO: 154 - DNA, 22 bp, Artificial sequence
  Synthetic
cagctaccatcatggtgaccaa

SEQ ID NO: 155 - DNA, 20 bp, Artificial sequence
  Synthetic
ggaggcagaaggctgttgaa

SEQ ID NO: 156 - DNA, 31 bp, Artificial sequence
  Synthetic
ccacactaccagggccaaatcctaactatgc

SEQ ID NO: 157 - DNA, 77 bp, Artificial sequence
  Synthetic
gtcctaattcctgattctgccaaacaagttattgagctggctggaaaacagcaaaccaccttcacggatcttgtggc

SEQ ID NO: 158 - DNA, 24 bp, Artificial sequence
  Synthetic
gtcctaattcctgattctgccaaa

SEQ ID NO: 159 - DNA, 20 bp, Artificial sequence
  Synthetic
gccacaagatccgtgaaggt

SEQ ID NO: 160 - DNA, 31 bp, Artificial sequence
  Synthetic
aagttattgagctggctggaaaacagcaaac

SEQ ID NO: 161 - DNA, 90 bp, Artificial sequence
  Synthetic
tgaggtcagagaaaataagacaggtcgaatgagtgatttgagtgtaattggtcatccaatagattcagaatctaaagaagatgaaccttg

SEQ ID NO: 162 - DNA, 24 bp, Artificial sequence
  Synthetic
tgaggtcagagaaaataagacagg

SEQ ID NO: 163 - DNA, 26 bp, Artificial sequence
  Synthetic
caaggttcatcttctttagattctga

SEQ ID NO: 164 - DNA, 31 bp, Artificial sequence
  Synthetic
cgaatgagtgatttgagtgtaattggtcatc

SEQ ID NO: 165 - DNA, 76 bp, Artificial sequence
  Synthetic
tgctaaaggctgcgaatgggatcctgatggaactaaggactccaatgtcgaactcttctttgctgcattccttttt

SEQ ID NO: 166 - DNA, 19 bp, Artificial sequence
  Synthetic
tgctaaaggctgcgaatgg

SEQ ID NO: 167 - DNA, 23 bp, Artificial sequence
  Synthetic
aaaaaggaatgcagcaaagaaga

SEQ ID NO: 168 - DNA, 30 bp, Artificial sequence
  Synthetic
cctgatggaactaaggactccaatgtcgaa

SEQ ID NO: 169 - DNA, 74 bp, Artificial sequence
  Synthetic
gagctgtggcttaccagatgtggcaaaatctggcaaaggcagagaccaaacttcatacctcaaactctgggggg

SEQ ID NO: 170 - DNA, 22 bp, Artificial sequence
  Synthetic
gagctgtggcttaccagatgtg

SEQ ID NO: 171 - DNA, 21 bp, Artificial sequence
  Synthetic
ccccccagagtttgaggtatg

SEQ ID NO: 172 - DNA, 28 bp, Artificial sequence
  Synthetic
caaaatctggcaaaggcagagaccaaac

SEQ ID NO: 173 - DNA, 73 bp, Artificial sequence
  Synthetic
agaggctgaagagctatgtgatcaccaccagcaggtgtccccagaaggctgtcatcttcagaaccaaactggg

SEQ ID NO: 174 - DNA, 24 bp, Artificial sequence
  Synthetic
agaggctgaagagctatgtgatca

SEQ ID NO: 175 - DNA, 23 bp, Artificial sequence
  Synthetic
cccagtttggttctgaagatgac

SEQ ID NO: 176 - DNA, 23 bp, Artificial sequence
  Synthetic
accagcaggtgtccccagaaggc

SEQ ID NO: 177 - DNA, 77 bp, Artificial sequence
  Synthetic
tggttctggggaagcattcaaatggatgcccgagctggagaaactatgtatttatatgaaaaggcaaatactcctga

SEQ ID NO: 178 - DNA, 19 bp, Artificial sequence
  Synthetic
tggttctggggaagcattc

SEQ ID NO: 179 - DNA, 24 bp, Artificial sequence
  Synthetic
tcaggagtatttgccttttcatat

SEQ ID NO: 180 - DNA, 24 bp, Artificial sequence
  Synthetic
aatggatgcccgagctggagaaac

SEQ ID NO: 181 - DNA, 76 bp, Artificial sequence
  Synthetic
gagctgcgtcatcacctacctggcccaggtggaccccaaaggctccttacccaagtgggtggtgaataaatcttct

SEQ ID NO: 182 - DNA, 21 bp, Artificial sequence
  Synthetic
gagctgcgtcatcacctacct

SEQ ID NO: 183 - DNA, 25 bp, Artificial sequence
  Synthetic
agaagatttattcaccacccacttg

SEQ ID NO: 184 - DNA, 23 bp, Artificial sequence
  Synthetic
ccaggtggaccccaaaggctcct

SEQ ID NO: 185 - DNA, 86 bp, Artificial sequence
  Synthetic
cggcccaccttcctgaagatcaagaagccactgtcgtaccgcaagcccatggacacggacctggtgtacatcgagaagtcgcccaa

SEQ ID NO: 186 - DNA, 17 bp, Artificial sequence
  Synthetic
cggcccaccttcctgaa

SEQ ID NO: 187 - DNA, 21 bp, Artificial sequence
  Synthetic
ttgggcgacttctcgatgtac

SEQ ID NO: 188 - DNA, 24 bp, Artificial sequence
  Synthetic
atcaagaagccactgtcgtaccgc

SEQ ID NO: 189 - DNA, 76 bp, Artificial sequence
  Synthetic
gctttcttccactgttgtctgaaccccatcctctatgctttccttggagccaaatttaaaacctctgcccagcacg

SEQ ID NO: 190 - DNA, 24 bp, Artificial sequence
  Synthetic
gctttcttccactgttgtctgaac

SEQ ID NO: 191 - DNA, 20 bp, Artificial sequence
  Synthetic
cgtgctgggcagaggtttta

SEQ ID NO: 192 - DNA, 27 bp, Artificial sequence
  Synthetic
ccatcctctatgctttccttggagcca

SEQ ID NO: 193 - DNA, 74 bp, Artificial sequence
  Synthetic
agctatgcaggtgacagagactcttgggatgacgcactgctgcatcaaccccatcatctatgcctttgtcgggg

SEQ ID NO: 194 - DNA, 25 bp, Artificial sequence
  Synthetic
agctatgcaggtgacagagactctt

SEQ ID NO: 195 - DNA, 20 bp, Artificial sequence
  Synthetic
ccccgacaaaggcatagatg

SEQ ID NO: 196 - DNA, 24 bp, Artificial sequence
  Synthetic
atgacgcactgctgcatcaacccc

SEQ ID NO: 197 - DNA, 71 bp, Artificial sequence
  Synthetic
gaccggtacatcgccattgtacaggcgactaagtcattccggctccgatccagaacactaccgcgcagcaa

SEQ ID NO: 198 - DNA, 20 bp, Artificial sequence
  Synthetic
gaccggtacatcgccattgt

SEQ ID NO: 199 - DNA, 19 bp, Artificial sequence
  Synthetic
ttgctgcgcggtagtgttc

SEQ ID NO: 200 - DNA, 25 bp, Artificial sequence
  Synthetic
caggcgactaagtcattccggctcc

SEQ ID NO: 201 - DNA, 71 bp, Artificial sequence
  Synthetic
gctcctggctgctttgatgtcagtgctgctactccacctctgcggcgaatcagaagcagcaagcaactttg

SEQ ID NO: 202 - DNA, 20 bp, Artificial sequence
  Synthetic
gctcctggctgctttgatgt

SEQ ID NO: 203 - DNA, 22 bp, Artificial sequence
  Synthetic
caaagttgcttgctgcttctga

SEQ ID NO: 204 - DNA, 22 bp, Artificial sequence
  Synthetic
tgctactccacctctgcggcga

SEQ ID NO: 205 - DNA, 71 bp, Artificial sequence
  Synthetic
tatgccatgaccagctttcactacaccatcatggtgacagaggccatcgcatacctgagggcctgccttaa

SEQ ID NO: 206 - DNA, 22 bp, Artificial sequence
  Synthetic
tatgccatgaccagctttcact

SEQ ID NO: 207 - DNA, 20 bp, Artificial sequence
  Synthetic
ttaaggcaggccctcaggta

SEQ ID NO: 208 - DNA, 25 bp, Artificial sequence
  Synthetic
accatcatggtgacagaggccatcg

SEQ ID NO: 209 - DNA, 71 bp, Artificial sequence
  Synthetic
atgagcaaacgcatggacatcgccatccaagtcacagaaagcatcgcactctttcacagctgcctcaaccc

SEQ ID NO: 210 - DNA, 21 bp, Artificial sequence
  Synthetic
atgagcaaacgcatggacatc

SEQ ID NO: 211 - DNA, 20 bp, Artificial sequence
  Synthetic
gggttgaggcagctgtgaaa

SEQ ID NO: 212 - DNA, 26 bp, Artificial sequence
  Synthetic
ccatccaagtcacagaaagcatcgca

SEQ ID NO: 213 - DNA, 71 bp, Artificial sequence
  Synthetic
ccacccactgctgcatcaaccctctcctgtatgcgtttcttgatgggacatttagcaaatacctctgccgc

SEQ ID NO: 214 - DNA, 18 bp, Artificial sequence
  Synthetic
ccacccactgctgcatca

SEQ ID NO: 215 - DNA, 21 bp, Artificial sequence
  Synthetic
gcggcagaggtatttgctaaa

SEQ ID NO: 216 - DNA, 28 bp, Artificial sequence
  Synthetic
ccctctcctgtatgcgtttcttgatggg

SEQ ID NO: 217 - DNA, 85 bp, Artificial sequence
  Synthetic
gctggtggtggctctccttgtcattttccaggtatgcctgtgtcaagatgaggtcacggacgattacatcggagacaacaccaca

SEQ ID NO: 218 - DNA, 19 bp, Artificial sequence
  Synthetic
gctggtggtggctctcctt

SEQ ID NO: 219 - DNA, 22 bp, Artificial sequence
  Synthetic
tgtggtgttgtctccgatgtaa

SEQ ID NO: 220 - DNA, 28 bp, Artificial sequence
  Synthetic
cgtgacctcatcttgacacaggcatacc

SEQ ID NO: 221 - DNA, 84 bp, Artificial sequence
  Synthetic
attcccgccaaggttgtccgcagctaccggaagcaggaaccaagcttaggctgctccatcccagctatcctgttcttgccccgc

SEQ ID NO: 222 - DNA, 18 bp, Artificial sequence
  Synthetic
attcccgccaaggttgtc

SEQ ID NO: 223 - DNA, 19 bp, Artificial sequence
  Synthetic
gcggggcaagaacaggata

SEQ ID NO: 224 - DNA, 24 bp, Artificial sequence
  Synthetic
cgcagctaccggaagcaggaacca

SEQ ID NO: 225 - DNA, 77 bp, Artificial sequence
  Synthetic
cagcctgaaaaagggcaagaaatgcagcaagaccaagaaatcccccgaaccagtcaggtttacttacgctggatgtt

SEQ ID NO: 226 - DNA, 20 bp, Artificial sequence
  Synthetic
cagcctgaaaaagggcaaga

SEQ ID NO: 227 - DNA, 26 bp, Artificial sequence
  Synthetic
aacatccagcgtaagtaaacctgact

SEQ ID NO: 228 - DNA, 25 bp, Artificial sequence
  Synthetic
cagcaagaccaagaaatcccccgaa

SEQ ID NO: 229 - DNA, 86 bp, Artificial sequence
  Synthetic
cccagcaccgactactaccaagagctgcagagagacatttctgaaatggctgtctgtcagtgccgccgaaagaactacgggcagct

SEQ ID NO: 230 - DNA, 21 bp, Artificial sequence
  Synthetic
cccagcaccgactactaccaa

SEQ ID NO: 231 - DNA, 20 bp, Artificial sequence
  Synthetic
agctgcccgtagttctttcg

SEQ ID NO: 232 - DNA, 27 bp, Artificial sequence
  Synthetic
agagagacatttctgaaatggctgtct

SEQ ID NO: 233 - DNA, 74 bp, Artificial sequence
  Synthetic
gccaaaggcattggctacttcttcgttttgcagcccaaggttgtagatggtactccatgtagcccagattccac

SEQ ID NO: 234 - DNA, 21 bp, Artificial sequence
  Synthetic
gccaaaggcattggctacttc

SEQ ID NO: 235 - DNA, 21 bp, Artificial sequence
  Synthetic
gtggaatctgggctacatgga

SEQ ID NO: 236 - DNA, 28 bp, Artificial sequence
  Synthetic
cgttttgcagcccaaggttgtagatggt

SEQ ID NO: 237 - DNA, 76 bp, Artificial sequence
  Synthetic
gggcccttcattgtagatctgattaccttcacggaaggaagcggacgctcaccacgctatgccctctggttctgtg

SEQ ID NO: 238 - DNA, 22 bp, Artificial sequence
  Synthetic
gggcccttcattgtagatctga

SEQ ID NO: 239 - DNA, 21 bp, Artificial sequence
  Synthetic
cacagaaccagagggcatagc

SEQ ID NO: 240 - DNA, 23 bp, Artificial sequence
  Synthetic
accttcacggaaggaagcggacg

SEQ ID NO: 241 - DNA, 67 bp, Artificial sequence
  Synthetic
cagcgtgggtcacctctgtgaacatcactgactgcaagcctccctcaatttctggtgcagcccatca

SEQ ID NO: 242 - DNA, 19 bp, Artificial sequence
  Synthetic
cagcgtgggtcacctctgt

SEQ ID NO: 243 - DNA, 19 bp, Artificial sequence
  Synthetic
tgatgggctgcaccagaaa

SEQ ID NO: 244 - DNA, 27 bp, Artificial sequence
  Synthetic
aacatcactgactgcaagcctccctca

SEQ ID NO: 245 - DNA, 83 bp, Artificial sequence
  Synthetic
tggttaatagtgtgtgacgctcaaagttaatgtaaactggaaaggttgtgtgtcgttgctttttgtgttttggttaggcttgg

SEQ ID NO: 246 - DNA, 24 bp, Artificial sequence
  Synthetic
tggttaatagtgtgtgacgctcaa

SEQ ID NO: 247 - DNA, 23 bp, Artificial sequence
  Synthetic
ccaagcctaaccaaaacacaaaa

SEQ ID NO: 248 - DNA, 33 bp, Artificial sequence
  Synthetic
ttaatgtaaactggaaaggttgtgtgtcgttgc

SEQ ID NO: 249 - DNA, 77 bp, Artificial sequence
  Synthetic
ggtttctggtggcctctatgaatcccatgtagggtgcagaccgtactccatccctccctgtgagcaccacgtcaacg

SEQ ID NO: 250 - DNA, 22 bp, Artificial sequence
  Synthetic
ggtttctggtggcctctatgaa

SEQ ID NO: 251 - DNA, 19 bp, Artificial sequence
  Synthetic
cgttgacgtggtgctcaca

SEQ ID NO: 252 - DNA, 28 bp, Artificial sequence
  Synthetic
cccatgtagggtgcagaccgtactccat

SEQ ID NO: 253 - DNA, 72 bp, Artificial sequence
  Synthetic
gcccagttgcctgtcttttgtgaatgcaaaggcttacaatggcaacactgccctccatgttgctgccagctt

SEQ ID NO: 254 - DNA, 20 bp, Artificial sequence
  Synthetic
gcccagttgcctgtcttttg

SEQ ID NO: 255 - DNA, 19 bp, Artificial sequence
  Synthetic
aagctggcagcaacatgga

SEQ ID NO: 256 - DNA, 29 bp, Artificial sequence
  Synthetic
aatgcaaaggcttacaatggcaacactgc

SEQ ID NO: 257 - DNA, 74 bp, Artificial sequence
  Synthetic
gctgggctggaacgtgaactcagtagctgaacctgtctgacccggtcacgttcttggatcctcagaactctttg

SEQ ID NO: 258 - DNA, 18 bp, Artificial sequence
  Synthetic
gctgggctggaacgtgaa

SEQ ID NO: 259 - DNA, 25 bp, Artificial sequence
  Synthetic
caaagagttctgaggatccaagaac

SEQ ID NO: 260 - DNA, 28 bp, Artificial sequence
  Synthetic
tcagtagctgaacctgtctgacccggtc

SEQ ID NO: 261 - DNA, 75 bp, Artificial sequence
  Synthetic
cgtggtggagtgcaactcaaaattggacccaacaaagaccactctcctcaagatggcggactgtggcggcctccc

SEQ ID NO: 262 - DNA, 20 bp, Artificial sequence
  Synthetic
cgtggtggagtgcaactcaa

SEQ ID NO: 263 - DNA, 16 bp, Artificial sequence
  Synthetic
gggaggccgccacagt

SEQ ID NO: 264 - DNA, 29 bp, Artificial sequence
  Synthetic
caacaaagaccactctcctcaagatggcg

SEQ ID NO: 265 - DNA, 86 bp, Artificial sequence
  Synthetic
tgttggtgccttgtttaatggtctgacactactgattttggctctcatttcactcttcagtgttcctgttatttatgaacggcatc

SEQ ID NO: 266 - DNA, 22 bp, Artificial sequence
  Synthetic
tgttggtgccttgtttaatggt

SEQ ID NO: 267 - DNA, 24 bp, Artificial sequence
  Synthetic
gatgccgttcataaataacaggaa

SEQ ID NO: 268 - DNA, 35 bp, Artificial sequence
  Synthetic
tgacactactgattttggctctcatttcactcttc

SEQ ID NO: 269 - DNA, 71 bp, Artificial sequence
  Synthetic
cccgggagaatatcctggtttcacttggagctgtgggtgagggcttccacaactaccaccactcctttccc

SEQ ID NO: 270 - DNA, 20 bp, Artificial sequence
  Synthetic
cccgggagaatatcctggtt

SEQ ID NO: 271 - DNA, 22 bp, Artificial sequence
  Synthetic
gggaaaggagtggtggtagttg

SEQ ID NO: 272 - DNA, 24 bp, Artificial sequence
  Synthetic
cacttggagctgtgggtgagggct

SEQ ID NO: 273 - DNA, 80 bp, Artificial sequence
  Synthetic
actgacatttcactcattcgctattttgtcactgaggtgctggacgtcattgctcctccttatacctctgacttcgtgca

SEQ ID NO: 274 - DNA, 27 bp, Artificial sequence
  Synthetic
actgacatttcactcattcgctatttt

SEQ ID NO: 275 - DNA, 24 bp, Artificial sequence
  Synthetic
tgcacgaagtcagaggtataagga

SEQ ID NO: 276 - DNA, 26 bp, Artificial sequence
  Synthetic
cactgaggtgctggacgtcattgctc

SEQ ID NO: 277 - DNA, 76 bp, Artificial sequence
  Synthetic
tggttttaactcctctctgtaaagcattttgcattttccccaagctcctttctgaaagaagacccagtgcagagcg

SEQ ID NO: 278 - DNA, 27 bp, Artificial sequence
  Synthetic
tggttttaactcctctctgtaaagcat

SEQ ID NO: 279 - DNA, 20 bp, Artificial sequence
  Synthetic
cgctctgcactgggtcttct

SEQ ID NO: 280 - DNA, 27 bp, Artificial sequence
  Synthetic
ttgcattttccccaagctcctttctga

SEQ ID NO: 281 - DNA, 76 bp, Artificial sequence
  Synthetic
ctggatgttcagtgaaggcttttaaattctttcagaaagatcgcaaaatggcgctcattcaattgggatctgtgga

SEQ ID NO: 282 - DNA, 23 bp, Artificial sequence
  Synthetic
ctggatgttcagtgaaggctttt

SEQ ID NO: 283 - DNA, 22 bp, Artificial sequence
  Synthetic
tccacagatcccaattgaatga

SEQ ID NO: 284 - DNA, 29 bp, Artificial sequence
  Synthetic
aattctttcagaaagatcgcaaaatggcg

SEQ ID NO: 285 - DNA, 78 bp, Artificial sequence
  Synthetic
caggctgcaagcgatatgcctaggctcaagcgatctgcccacctcaacttccggaagtgctgagattacaggtgtgag

SEQ ID NO: 286 - DNA, 19 bp, Artificial sequence
  Synthetic
caggctgcaagcgatatgc

SEQ ID NO: 287 - DNA, 25 bp, Artificial sequence
  Synthetic
ctcacacctgtaatctcagcacttc

SEQ ID NO: 288 - DNA, 25 bp, Artificial sequence
  Synthetic
taggctcaagcgatctgcccacctc

SEQ ID NO: 289 - DNA, 79 bp, Artificial sequence
  Synthetic
ctaccagcaggaggctaaagagatattgtccagaggatcaactgaggccaaaattactgaggtcaaagtggaacctatg

SEQ ID NO: 290 - DNA, 23 bp, Artificial sequence
  Synthetic
ctaccagcaggaggctaaagaga

SEQ ID NO: 291 - DNA, 26 bp, Artificial sequence
  Synthetic
cataggttccactttgacctcagtaa

SEQ ID NO: 292 - DNA, 28 bp, Artificial sequence
  Synthetic
attgtccagaggatcaactgaggccaaa

SEQ ID NO: 293 - DNA, 78 bp, Artificial sequence
  Synthetic
gcactgaaatggaacaactcctttaaacgtgcagccttttgaatttttcctcaaaaccaagaagttgacctctgagct

SEQ ID NO: 294 - DNA, 23 bp, Artificial sequence
  Synthetic
gcactgaaatggaacaactcctt

SEQ ID NO: 295 - DNA, 24 bp, Artificial sequence
  Synthetic
agctcagaggtcaacttcttggtt

SEQ ID NO: 296 - DNA, 29 bp, Artificial sequence
  Synthetic
aaacgtgcagccttttgaatttttcctca

SEQ ID NO: 297 - DNA, 81 bp, Artificial sequence
  Synthetic
ggtgttcttcaagaaagtttctcaaaacagggagtacttcaagatcaccctctacgggagaaccaaggagctgacttcgga

SEQ ID NO: 298 - DNA, 26 bp, Artificial sequence
  Synthetic
ggtgttcttcaagaaagtttctcaaa

SEQ ID NO: 299 - DNA, 20 bp, Artificial sequence
  Synthetic
tccgaagtcagctccttggt

SEQ ID NO: 300 - DNA, 32 bp, Artificial sequence
  Synthetic
agggagtacttcaagatcaccctctacgggag

SEQ ID NO: 301 - DNA, 73 bp, Artificial sequence
  Synthetic
aaacaccagttcctggatggattcccgccatccatgccccctctttaattagccggtcctctcaataatgtag

SEQ ID NO: 302 - DNA, 22 bp, Artificial sequence
  Synthetic
aaacaccagttcctggatggat

SEQ ID NO: 303 - DNA, 25 bp, Artificial sequence
  Synthetic
ctacattattgagaggaccggctaa

SEQ ID NO: 304 - DNA, 18 bp, Artificial sequence
  Synthetic
cccgccatccatgccccc

SEQ ID NO: 305 - DNA, 73 bp, Artificial sequence
  Synthetic
aacgctgtgcctggcagtatggctaaccgccattatggctaccatcccattgctagtgttttaccaagtggcc

SEQ ID NO: 306 - DNA, 20 bp, Artificial sequence
  Synthetic
aacgctgtgcctggcagtat

SEQ ID NO: 307 - DNA, 23 bp, Artificial sequence
  Synthetic
ggccacttggtaaaacactagca

SEQ ID NO: 308 - DNA, 27 bp, Artificial sequence
  Synthetic
ctaaccgccattatggctaccatccca

SEQ ID NO: 309 - DNA, 73 bp, Artificial sequence
  Synthetic
cagagagccaggagcatcctgaagctgacccaggtagcgctgccccatacctgaagaccaagtttatctgtgt

SEQ ID NO: 310 - DNA, 20 bp, Artificial sequence
  Synthetic
cagagagccaggagcatcct

SEQ ID NO: 311 - DNA, 28 bp, Artificial sequence
  Synthetic
acacagataaacttggtcttcaggtatg

SEQ ID NO: 312 - DNA, 23 bp, Artificial sequence
  Synthetic
aagctgacccaggtagcgctgcc

SEQ ID NO: 313 - DNA, 68 bp, Artificial sequence
  Synthetic
ctgccaggaccgcttctactggcgcgtgagttcccggagtgagttgaaccaggtggaccaagtgggct

SEQ ID NO: 314 - DNA, 20 bp, Artificial sequence
  Synthetic
ctgccaggaccgcttctact

SEQ ID NO: 315 - DNA, 19 bp, Artificial sequence
  Synthetic
agcccacttggtccacctg

SEQ ID NO: 316 - DNA, 25 bp, Artificial sequence
  Synthetic
gcgtgagttcccggagtgagttgaa

SEQ ID NO: 317 - DNA, 67 bp, Artificial sequence
  Synthetic
cagagcgactactccagctatgcccggacctccatcagagccagtctcaccttcaaccgcggcttca

SEQ ID NO: 318 - DNA, 23 bp, Artificial sequence
  Synthetic
cagagcgactactccagctatgc

SEQ ID NO: 319 - DNA, 17 bp, Artificial sequence
  Synthetic
tgaagccgcggttgaag

SEQ ID NO: 320 - DNA, 25 bp, Artificial sequence
  Synthetic
cggacctccatcagagccagtctca

SEQ ID NO: 321 - DNA, 74 bp, Artificial sequence
  Synthetic
tgaatcacatcgagccactgaaaattcagcttcagcgagaacccagacctttcccaaagctcaggattcttcga

SEQ ID NO: 322 - DNA, 21 bp, Artificial sequence
  Synthetic
tgaatcacatcgagccactga

SEQ ID NO: 323 - DNA, 21 bp, Artificial sequence
  Synthetic
tcgaagaatcctgagctttgg

SEQ ID NO: 324 - DNA, 27 bp, Artificial sequence
  Synthetic
aattcagcttcagcgagaacccagacc

SEQ ID NO: 325 - DNA, 79 bp, Artificial sequence
  Synthetic
tggaggggaggctaactgtgccctgaagctgagcctggaatgcagggtcgacctcctcttcctgctggacagctctgcg

SEQ ID NO: 326 - DNA, 20 bp, Artificial sequence
  Synthetic
tggaggggaggctaactgtg

SEQ ID NO: 327 - DNA, 18 bp, Artificial sequence
  Synthetic
cgcagagctgtccagcag

SEQ ID NO: 328 - DNA, 24 bp, Artificial sequence
  Synthetic
ccctgaagctgagcctggaatgca

SEQ ID NO: 329 - DNA, 74 bp, Artificial sequence
  Synthetic
cagggtttcaggttccaatcagaactgttggcaaggtgacatttccatgcataaatgcgatccacagaaggtcc

SEQ ID NO: 330 - DNA, 21 bp, Artificial sequence
  Synthetic
cagggtttcaggttccaatca

SEQ ID NO: 331 - DNA, 21 bp, Artificial sequence
  Synthetic
ggaccttctgtggatcgcatt

SEQ ID NO: 332 - DNA, 28 bp, Artificial sequence
  Synthetic
ctgttggcaaggtgacatttccatgcat

SEQ ID NO: 333 - DNA, 80 bp, Artificial sequence
  Synthetic
aagcttgtgaggagacggactctatgttgcccaggctgttatggaactcctgagtcaagtgatcctcccaccttggcctc

SEQ ID NO: 334 - DNA, 23 bp, Artificial sequence
  Synthetic
aagcttgtgaggagacggactct

SEQ ID NO: 335 - DNA, 19 bp, Artificial sequence
  Synthetic
gaggccaaggtgggaggat

SEQ ID NO: 336 - DNA, 27 bp, Artificial sequence
  Synthetic
tgttgcccaggctgttatggaactcct

SEQ ID NO: 337 - DNA, 64 bp, Artificial sequence
  Synthetic
tacctccaccatgccaagtggtcccaggctgcacccatggcagaaggaggagggcagaatcatc

SEQ ID NO: 338 - DNA, 20 bp, Artificial sequence
  Synthetic
tacctccaccatgccaagtg

SEQ ID NO: 339 - DNA, 21 bp, Artificial sequence
  Synthetic
gatgattctgccctcctcctt

SEQ ID NO: 340 - DNA, 20 bp, Artificial sequence
  Synthetic
tcccaggctgcacccatggc

SEQ ID NO: 341 - DNA, 73 bp, Artificial sequence
  Synthetic
ctggccgcaataattccatagtcaagagcatcacagtctctgcatctggaacttctcctggtctctcagctgg

SEQ ID NO: 342 - DNA, 21 bp, Artificial sequence
  Synthetic
ctggccgcaataattccatag

SEQ ID NO: 343 - DNA, 21 bp, Artificial sequence
  Synthetic
ccagctgagagaccaggagaa

SEQ ID NO: 344 - DNA, 29 bp, Artificial sequence
  Synthetic
caagagcatcacagtctctgcatctggaa

SEQ ID NO: 345 - DNA, 83 bp, Artificial sequence
  Synthetic
tcccagagccttgagatagaactccagtcccatctcagcatgaaagagtctttggagcacactctagaggagaccaaggcccg

SEQ ID NO: 346 - DNA, 24 bp, Artificial sequence
  Synthetic
tcccagagccttgagatagaactc

SEQ ID NO: 347 - DNA, 20 bp, Artificial sequence
  Synthetic
cgggccttggtctcctctag

SEQ ID NO: 348 - DNA, 30 bp, Artificial sequence
  Synthetic
tctcagcatgaaagagtctttggagcacac

SEQ ID NO: 349 - DNA, 72 bp, Artificial sequence
  Synthetic
caagcgaactggcattccaaccctagggaagaatgtggttgtggctggaaggtcaaaaaacgttggaatgcc

SEQ ID NO: 350 - DNA, 19 bp, Artificial sequence
  Synthetic
caagcgaactggcattcca

SEQ ID NO: 351 - DNA, 21 bp, Artificial sequence
  Synthetic
ggcattccaacgttttttgac

SEQ ID NO: 352 - DNA, 28 bp, Artificial sequence
  Synthetic
cctagggaagaatgtggttgtggctgga

SEQ ID NO: 353 - DNA, 71 bp, Artificial sequence
  Synthetic
gggaagccgcctataccaagaggctcggcacagagaaggaggtcgaggtgattgggggagcagacaagtac

SEQ ID NO: 354 - DNA, 19 bp, Artificial sequence
  Synthetic
gggaagccgcctataccaa

SEQ ID NO: 355 - DNA, 22 bp, Artificial sequence
  Synthetic
gtacttgtctgctcccccaatc

SEQ ID NO: 356 - DNA, 24 bp, Artificial sequence
  Synthetic
tcggcacagagaaggaggtcgagg

SEQ ID NO: 357 - DNA, 76 bp, Artificial sequence
  Synthetic
gtttccaccagctcatgaaagacacgactttgaactggaagatggtggatgcttccaaaagcatcgaagctgtcca

SEQ ID NO: 358 - DNA, 23 bp, Artificial sequence
  Synthetic
gtttccaccagctcatgaaagac

SEQ ID NO: 359 - DNA, 21 bp, Artificial sequence
  Synthetic
tggacagcttcgatgcttttg

SEQ ID NO: 360 - DNA, 28 bp, Artificial sequence
  Synthetic
cgactttgaactggaagatggtggatgc

SEQ ID NO: 361 - DNA, 81 bp, Artificial sequence
  Synthetic
gcagtttgcattgcagtcaacagtcgaagaaggtgtgggcagaagaaaaagctagtgatcaacagtggcaatggagctgtg

SEQ ID NO: 362 - DNA, 21 bp, Artificial sequence
  Synthetic
gcagtttgcattgcagtcaac

SEQ ID NO: 363 - DNA, 21 bp, Artificial sequence
  Synthetic
cacagctccattgccactgtt

SEQ ID NO: 364 - DNA, 32 bp, Artificial sequence
  Synthetic
agaaggtgtgggcagaagaaaaagctagtgat

SEQ ID NO: 365 - DNA, 81 bp, Artificial sequence
  Synthetic
ccgtggagacttctgcatacaagttggcaggaacattatacatggcagtgattctgtggagagtgcagagaaggagatcgg

SEQ ID NO: 366 - DNA, 23 bp, Artificial sequence
  Synthetic
ccgtggagacttctgcatacaag

SEQ ID NO: 367 - DNA, 22 bp, Artificial sequence
  Synthetic
ccgatctccttctctgcactct

SEQ ID NO: 368 - DNA, 31 bp, Artificial sequence
  Synthetic
tggcaggaacattatacatggcagtgattct

SEQ ID NO: 369 - DNA, 79 bp, Artificial sequence
  Synthetic
tctctatccttgccgacaactgccccttcaaactcttccagcctcaccctgtcaggtatcaaagaagacaacagcttgc

SEQ ID NO: 370 - DNA, 22 bp, Artificial sequence
  Synthetic
tctctatccttgccgacaactg

SEQ ID NO: 371 - DNA, 25 bp, Artificial sequence
  Synthetic
gcaagctgttgtcttctttgatacc

SEQ ID NO: 372 - DNA, 27 bp, Artificial sequence
  Synthetic
ccttcaaactcttccagcctcaccctg

SEQ ID NO: 373 - DNA, 77 bp, Artificial sequence
  Synthetic
ctttgcacagaaccagctagttatttggaagtacccaaccatggccaaggtggctgaactcaaaggtcacacatccc

SEQ ID NO: 374 - DNA, 26 bp, Artificial sequence
  Synthetic
ctttgcacagaaccagctagttattt

SEQ ID NO: 375 - DNA, 23 bp, Artificial sequence
  Synthetic
gggatgtgtgacctttgagttca

SEQ ID NO: 376 - DNA, 25 bp, Artificial sequence
  Synthetic
aagtacccaaccatggccaaggtgg

SEQ ID NO: 377 - DNA, 77 bp, Artificial sequence
  Synthetic
caattacatttcctgcaccaacaaggccaacctgtcccaagtggccgaccatctggatcacatcaaggaggtggcag

SEQ ID NO: 378 - DNA, 24 bp, Artificial sequence
  Synthetic
caattacatttcctgcaccaacaa

SEQ ID NO: 379 - DNA, 21 bp, Artificial sequence
  Synthetic
ctgccacctccttgatgtgat

SEQ ID NO: 380 - DNA, 24 bp, Artificial sequence
  Synthetic
cctgtcccaagtggccgaccatct

SEQ ID NO: 381 - DNA, 75 bp, Artificial sequence
  Synthetic
ttattcttttgggcacctttggttgttttgctacctgccgagcttctgcatggatgctaaaactgtatgcaatgt

SEQ ID NO: 382 - DNA, 22 bp, Artificial sequence
  Synthetic
ttattcttttgggcacctttgg

SEQ ID NO: 383 - DNA, 25 bp, Artificial sequence
  Synthetic
acattgcatacagttttagcatcca

SEQ ID NO: 384 - DNA, 26 bp, Artificial sequence
  Synthetic
tgttttgctacctgccgagcttctgc

SEQ ID NO: 385 - DNA, 78 bp, Artificial sequence
  Synthetic
ctgaacgggagccgtagctcaggaggcagaattcagtgtgttatcattgggcagaactggatcctgaaaaattcaaga

SEQ ID NO: 386 - DNA, 19 bp, Artificial sequence
  Synthetic
ctgaacgggagccgtagct

SEQ ID NO: 387 - DNA, 26 bp, Artificial sequence
  Synthetic
tcttgaatttttcaggatccagttct

SEQ ID NO: 388 - DNA, 31 bp, Artificial sequence
  Synthetic
aggaggcagaattcagtgtgttatcattggg

SEQ ID NO: 389 - DNA, 78 bp, Artificial sequence
  Synthetic
ttatcaatgagaagcgcaacatccacgcagctgctcatcaggtacgagtgcttccatatccatttacccaccattggc

SEQ ID NO: 390 - DNA, 23 bp, Artificial sequence
  Synthetic
ttatcaatgagaagcgcaacatc

SEQ ID NO: 391 - DNA, 21 bp, Artificial sequence
  Synthetic
gccaatggtgggtaaatggat

SEQ ID NO: 392 - DNA, 28 bp, Artificial sequence
  Synthetic
cagctgctcatcaggtacgagtgcttcc

SEQ ID NO: 393 - DNA, 71 bp, Artificial sequence
  Synthetic
gtggcagaacttccagaggaaatcgacatctccacatacatcgagcagagccggtaaccaccacgtgccag

SEQ ID NO: 394 - DNA, 22 bp, Artificial sequence
  Synthetic
gtggcagaacttccagaggaaa

SEQ ID NO: 395 - DNA, 19 bp, Artificial sequence
  Synthetic
ctggcacgtggtggttacc

SEQ ID NO: 396 - DNA, 28 bp, Artificial sequence
  Synthetic
cgacatctccacatacatcgagcagagc

SEQ ID NO: 397 - DNA, 71 bp, Artificial sequence
  Synthetic
gcctgagccagccagtgacagcgtttcctcagggcacttggattggtgaagacatattccctcctctgctg

SEQ ID NO: 398 - DNA, 18 bp, Artificial sequence
  Synthetic
gcctgagccagccagtga

SEQ ID NO: 399 - DNA, 24 bp, Artificial sequence
  Synthetic
cagcagaggagggaatatgtcttc

SEQ ID NO: 400 - DNA, 26 bp, Artificial sequence
  Synthetic
agcgtttcctcagggcacttggattg

SEQ ID NO: 401 - DNA, 87 bp, Artificial sequence
  Synthetic
gcgagaagatgacccagatcatgtttgagaccttcaacaccccagccatgtacgttgctatccaggctgtgctatccctgtacgcct

SEQ ID NO: 402 - DNA, 21 bp, Artificial sequence
  Synthetic
gcgagaagatgacccagatca

SEQ ID NO: 403 - DNA, 20 bp, Artificial sequence
  Synthetic
aggcgtacagggatagcaca

SEQ ID NO: 404 - DNA, 25 bp, Artificial sequence
  Synthetic
accttcaacaccccagccatgtacg

SEQ ID NO: 405 - DNA, 86 bp, Artificial sequence
  Synthetic
gctgtgatgtgaagtttcctataaggttagaaggccttgtgctcacccaccaacaatttagtagttatgagccagagttatttcct

SEQ ID NO: 406 - DNA, 26 bp, Artificial sequence
  Synthetic
gctgtgatgtgaagtttcctataagg

SEQ ID NO: 407 - DNA, 31 bp, Artificial sequence
  Synthetic
aggaaataactctggctcataactactaaat

SEQ ID NO: 408 - DNA, 24 bp, Artificial sequence
  Synthetic
aaggccttgtgctcacccaccaac

SEQ ID NO: 409 - DNA, 83 bp, Artificial sequence
  Synthetic
gctgctttccctttccttgcatattctggaatcccagcagtttctttctgtttttgcgaggacacagattatccttatttggg

SEQ ID NO: 410 - DNA, 19 bp, Artificial sequence
  Synthetic
gctgctttccctttccttg

SEQ ID NO: 411 - DNA, 24 bp, Artificial sequence
  Synthetic
cccaaataaggataatctgtgtcc

SEQ ID NO: 412 - DNA, 27 bp, Artificial sequence
  Synthetic
cgcaaaaacagaaagaaactgctggga

SEQ ID NO: 413 - DNA, 73 bp, Artificial sequence
  Synthetic
tttccttggtcaggcagtataatccaaagatggtcaaggtcgcaagcttgctggtgaaaaggaccccacgaag

SEQ ID NO: 414 - DNA, 23 bp, Artificial sequence
  Synthetic
tttccttggtcaggcagtataat

SEQ ID NO: 415 - DNA, 20 bp, Artificial sequence
  Synthetic
cttcgtggggtccttttcac

SEQ ID NO: 416 - DNA, 24 bp, Artificial sequence
  Synthetic
ccaaagatggtcaaggtcgcaagc

SEQ ID NO: 417 - DNA, 72 bp, Artificial sequence
  Synthetic
ctttgtcacagcccaagatagttaagtgggatcgagacatgtaagcagcatcatggaggtttgaagatgccg

SEQ ID NO: 418 - DNA, 23 bp, Artificial sequence
  Synthetic
ctttgtcacagcccaagatagtt

SEQ ID NO: 419 - DNA, 20 bp, Artificial sequence
  Synthetic
cggcatcttcaaacctccat

SEQ ID NO: 420 - DNA, 28 bp, Artificial sequence
  Synthetic
aagtgggatcgagacatgtaagcagcat

SEQ ID NO: 421 - DNA, 81 bp, Artificial sequence
  Synthetic
ccagctccctgcgaagagcagcccagctgcagagaaagttcccgcatctggagttcaggagtattcggggaaacctcaaca

SEQ ID NO: 422 - DNA, 18 bp, Artificial sequence
  Synthetic
ccagctccctgcgaagag

SEQ ID NO: 423 - DNA, 22 bp, Artificial sequence
  Synthetic
tgttgaggtttccccgaatact

SEQ ID NO: 424 - DNA, 26 bp, Artificial sequence
  Synthetic
tgcagagaaagttcccgcatctggag

SEQ ID NO: 425 - DNA, 64 bp, Artificial sequence
  Synthetic
ggcaatgcggctgcaacggcggaagaaaacagcccaaagatgagagtgattcgcgtgggtaccc

SEQ ID NO: 426 - DNA, 16 bp, Artificial sequence
  Synthetic
ggcaatgcggctgcaa

SEQ ID NO: 427 - DNA, 19 bp, Artificial sequence
  Synthetic
gggtacccacgcgaatcac

SEQ ID NO: 428 - DNA, 27 bp, Artificial sequence
  Synthetic
tctcatctttgggctgttttcttccgc

SEQ ID NO: 429 - DNA, 84 bp, Artificial sequence
  Synthetic
aggcgcggaaggagtcacggggcgcgcatgccagggaagactacaaggtgcggattgatgagtacgattactccaagcccatcc

SEQ ID NO: 430 - DNA, 17 bp, Artificial sequence
  Synthetic
aggcgcggaaggagtca

SEQ ID NO: 431 - DNA, 20 bp, Artificial sequence
  Synthetic
ggatgggcttggagtaatcg

SEQ ID NO: 432 - DNA, 29 bp, Artificial sequence
  Synthetic
agggaagactacaaggtgcggattgatga

SEQ ID NO: 433 - DNA, 80 bp, Artificial sequence
  Synthetic
tgggaacaagagggcatctgctaaagtttcagattccatttctgctcagtatccagtagtggatcatgaatttgatgcag

SEQ ID NO: 434 - DNA, 20 bp, Artificial sequence
  Synthetic
tgggaacaagagggcatctg

SEQ ID NO: 435 - DNA, 22 bp, Artificial sequence
  Synthetic
ccaccactgcatcaaattcatg

SEQ ID NO: 436 - DNA, 32 bp, Artificial sequence
  Synthetic
ccactactggatactgagcagaaatggaatct

SEQ ID NO: 437 - DNA, 73 bp, Artificial sequence
  Synthetic
tcgcagccgggatttgggtcgcggttcttgtttgtggatcgctgtgatcgtcacttgacaatgcagatcttcg

SEQ ID NO: 438 - DNA, 16 bp, Artificial sequence
  Synthetic
tcgcagccgggatttg

SEQ ID NO: 439 - DNA, 23 bp, Artificial sequence
  Synthetic
cgaagatctgcattgtcaagtga

SEQ ID NO: 440 - DNA, 24 bp, Artificial sequence
  Synthetic
tcgcggttcttgtttgtggatcgc

SEQ ID NO: 441 - DNA, 86 bp, Artificial sequence
  Synthetic
agggctgcttttaactctggtaaagtggatattgttgccatcaatgaccccttcattgacctcaactacatggtttacatgttcca

SEQ ID NO: 442 - DNA, 24 bp, Artificial sequence
  Synthetic
agggctgcttttaactctggtaaa

SEQ ID NO: 443 - DNA, 26 bp, Artificial sequence
  Synthetic
tggaacatgtaaaccatgtagttgag

SEQ ID NO: 444 - DNA, 28 bp, Artificial sequence
  Synthetic
tggatattgttgccatcaatgacccctt

SEQ ID NO: 445 - DNA, 74 bp, Artificial sequence
  Synthetic
ggaattttgccgatttcatgactgaacagtcaccgacgagagtgctggggaataaaaaggggatcttcactcgg

SEQ ID NO: 446 - DNA, 21 bp, Artificial sequence
  Synthetic
ggaattttgccgatttcatga

SEQ ID NO: 447 - DNA, 21 bp, Artificial sequence
  Synthetic
ccgagtgaagatccccttttt

SEQ ID NO: 448 - DNA, 26 bp, Artificial sequence
  Synthetic
tgaacagtcaccgacgagagtgctgg

SEQ ID NO: 449 - DNA, 74 bp, Artificial sequence
  Synthetic
gcagcatctacaaccctgaagtgcttgatatcacagaggaaactctgcattctcgcttcctggagggtgtccgc

SEQ ID NO: 450 - DNA, 22 bp, Artificial sequence
  Synthetic
gcagcatctacaaccctgaagt

SEQ ID NO: 451 - DNA, 17 bp, Artificial sequence
  Synthetic
gcggacaccctccagga

SEQ ID NO: 452 - DNA, 31 bp, Artificial sequence
  Synthetic
ttgatatcacagaggaaactctgcattctcg

SEQ ID NO: 453 - DNA, 76 bp, Artificial sequence
  Synthetic
tggaggagaagaggaaagagaaagccaagatccactaccggaagaagaaacagctcatgaggctacggaaacaggc

SEQ ID NO: 454 - DNA, 23 bp, Artificial sequence
  Synthetic
tggaggagaagaggaaagagaaa

SEQ ID NO: 455 - DNA, 20 bp, Artificial sequence
  Synthetic
gcctgtttccgtagcctcat

SEQ ID NO: 456 - DNA, 28 bp, Artificial sequence
  Synthetic
ccaagatccactaccggaagaagaaaca

SEQ ID NO: 457 - DNA, 78 bp, Artificial sequence
  Synthetic
gcttctaggagataaaaagaacatccagtcatggataaaaatgagctggttcagaaggccaaactggccgagcaggct

SEQ ID NO: 458 - DNA, 27 bp, Artificial sequence
  Synthetic
gcttctaggagataaaaagaacatcca

SEQ ID NO: 459 - DNA, 17 bp, Artificial sequence
  Synthetic
agcctgctcggccagtt

SEQ ID NO: 460 - DNA, 29 bp, Artificial sequence
  Synthetic
tggataaaaatgagctggttcagaaggcc

SEQ ID NO: 461 - DNA, 81 bp, Artificial sequence
  Synthetic
agcgggcgcagtgattataggctttcgctctaagattaaaaatgccctagcccacttcttaccacaaggcacacctacacc

SEQ ID NO: 462 - DNA, 20 bp, Artificial sequence
  Synthetic
agcgggcgcagtgattatag

SEQ ID NO: 463 - DNA, 22 bp, Artificial sequence
  Synthetic
ggtgtaggtgtgccttgtggta

SEQ ID NO: 464 - DNA, 33 bp, Artificial sequence
  Synthetic
cgctctaagattaaaaatgccctagcccacttc